(19) United States
(12) Patent Application Publication
(10) Pub. No.: US 2024/0287535 A1
(43) Pub. Date: Aug. 29, 2024

(54) DELAY OR PREVENTION OF BROWNING IN BANANA FRUIT

(71) Applicant: Tropic Biosciences UK Limited, Norwich, Norfolk (GB)

(72) Inventors: Daniel KNEVITT, Norwich, Norfolk (GB); Robert Tristan GREEN, Norwich, Norfolk (GB); Silvia Marina PAIS DIEGUEZ, Norwich, Norfolk (GB); David GUILLAUME-SCHOEPFER, Norwich, Norfolk (GB); Angela Lucia CHAPARRO GARCIA, Norwich, Norfolk (GB)

(73) Assignee: Tropic Biosciences UK Limited, Norwich, Norfolk (GB)

(21) Appl. No.: 18/571,140

(22) PCT Filed: Jun. 30, 2022
(86) PCT No.: PCT/EP2022/068072

§ 371 Date: Dec. 15, 2023

(30) Foreign Application Priority Data
Jul. 2, 2021 (GB) 2109585.6
Nov. 12, 2021 (GB) 2116382.9

(51) Int. Cl. C12N 15/82; C12N 9/22; C12N 15/11; C12Q 1/6851; C12Q 1/6895

(52) U.S. Cl. CPC C12N 15/8262; C12N 9/22; C12N 15/11; C12N 15/8213; C12Q 1/6851; C12Q 1/6895; C12Y 110/03001; C12N 2310/20; C12N 2800/80; C12Q 2600/13

(57) ABSTRACT

The present invention relates to compositions and methods for the delay or prevention of browning in banana fruit by genetically editing one or more genes encoding a Polyphenol Oxidase (PPO).

### FIELD OF THE INVENTION

The present invention, according to some embodiments, relates to compositions and methods for the delay or prevention of browning in banana fruit. In some embodiments of the present invention, the delay or prevention of browning in banana fruit is achieved by genetically editing one or more genes encoding a Polyphenol Oxidase (PPO).

### BACKGROUND OF THE INVENTION

Cultivated bananas and plantains are giant herbaceous plants within the genus Musa. They are both sterile and parthenocarpic, so the fruit develops without seed. Cultivated hybrids and species are mostly triploid, although some are diploid or tetraploid. Most have been propagated from mutants found in the wild.

Banana belongs to a climacteric fruit. After harvesting, green banana has to undergo climacteric change through its ripening process (including production of internal ethylene, hydrolysis of starch and protopectin), until the fruit flesh softens, sweetness increases, and a fragrance is produced, thus increasing dietary value. Conventionally, banana is harvested in advance of ripening, and thus the duration of its transportation and storage period is affected by the length of the ripening progress. Banana fruit may often undergo ripening due to the production of ethylene during the transportation process. Furthermore, the fruit may be over-ripened, start to brown, and become spoiled, thus lowering its market value. There is therefore a need to control fruit browning in banana. In particular, the ability to delay or prevent fruit browning would facilitate the transport of banana fruit and would improve banana fruit shelf life. One of the elements which may affect fruit browning are polyphenol oxidases (PPOs).

Polyphenol oxidases (PPOs) are enzymes found throughout the plant and animal kingdoms, including in most fruits and vegetables, such as banana. PPOs are important in the food industry because they catalyse enzymatic browning when tissues are damaged from bruising or compression, making the produce less marketable and causing economic loss. Enzymatic browning due to PPO action can also lead to loss of nutritional content of the produce, further lowering its value. Because the substrates of PPO reactions are located in the vacuoles of plant cells, PPOs initiate the chain of browning reactions. Exposure to oxygen when fruit it sliced or pureed also leads to enzymatic browning by PPOs. PPOs are known to accept monophenols and/or o-diphenols as substrates, and work by catalyzing the o-hydroxylation of monophenol molecules in which the benzene ring contains a single hydroxyl substituent to o-diphenols (phenol molecules containing two hydroxyl substituents at the 1, 2 positions, with no carbon between). The enzymes can also further catalyse the oxidation of o-diphenols to produce o-quinones. PPOs catalyse the rapid polymerization of o-quinones to produce black, brown or red pigments (polyphenols) that cause fruit browning. Accordingly, it would be desirable to find a way to reduce the level or activity of PPOs active in fruit in an effort to delay or prevent fruit browning, for example in banana.

Reducing the expression of PPO genes to reduce PPO level or activity is one way of preventing fruit browning (as has been described in U.S. Pat. No. 9,580,723 for apple, so-called “arctic apple”). Typical approaches to improve agricultural productivity (such as enhanced yield or engineered pest resistance) have previously relied on either mutation breeding or introduction of genes into the genomes of crop species by transformation. However, these processes are inherently nonspecific and relatively inefficient. For example, plant transformation methods deliver exogenous DNA that integrates into the genome at random locations. Furthermore, the random nature of these integrations makes it difficult to predict whether pleiotropic effects due to unintended genome disruption have occurred. Recent advances in genome editing techniques have made it possible to alter DNA sequences in living cells in a manner which is more precise than conventional breeding or standard genetic engineering, such as by the use of CRISPR-Cas9 gene editing.

However, unlike most other major food crops, bananas are difficult to genetically improve. This is partly because banana species are parthenocarpic (do not produce viable seeds), and so the removal of a genetically inserted sequences by sexual reproduction is impossible (e.g. removal of transfer DNA, T-DNA, inserted using Agrobacteria introducing the Cas9 sequence). In addition, since nearly all banana cultivars and landraces are triploids, with high levels of male and female infertility, it is impossible to backcross bananas, thus excluding the possibility of introgressing new traits into current cultivars. Moreover, the incomplete annotation of the banana genome and limited expression data does not provide sufficient depth of information as to the best genes to target.

The present invention is based in part on the identification of nine different PPO genes in banana (termed PPO1-PPO9), and in particular the characterisation of specific PPOs, such as PPO1, PPO2, PPO8, PPO9, and PPO4 as being expressed in banana fruit, but characterised by lower or absent expression in other tissues. According to some embodiments of the present invention, the identified PPO genes, and in particular PPO1, PPO2, PPO8, PPO9, and/or PPO4 are targeted herein to delay or prevent browning in banana fruit. Previously, Gooding et al., “Molecular cloning and characterisation of banana fruit polyphenol oxidase”, Planta, September 2001; 123(5): 748-57, used degenerate primers to identify banana PPOs, which do not match any of the PPOs herein, but most closely correlate with either PPO4 and PPO5. WO9637617, WO9729193 and WO9853080 used the same degenerate primers of Gooding et al. (2001) infra. CN104404007 discloses the discovery of a PPO gene in banana, which is referred to as PPO8 herein.

### SUMMARY OF THE INVENTION

The invention provides a method of reducing the level or activity of at least one endogenous PPO1, PPO2, PPO8, PPO9, or PPO4 polyphenol oxidase encoded by a PPO1, PPO2, PPO8, PPO9, or PPO4 polyphenol oxidase gene in a banana plant or banana plant cell. Optionally, the method of the invention further comprises regenerating a banana plant from said banana plant cell. Further optionally, the method of the invention further comprises harvesting fruit from said banana plant. According to some embodiments, reducing the level or activity of at least one endogenous PPO1, PPO2, PPO8, PPO9, or PPO4 polyphenol oxidase comprises introducing a modification into the PPO1, PPO2, PPO8, PPO9, or PPO4 polyphenol oxidase gene encoding said at least one PPO1, PPO2, PPO8, PPO9, or PPO4 polyphenol oxidase.

The invention further provides a banana plant cell obtainable by the above method of the invention, a banana plant or plant part obtainable by the above method of the invention, and fruit harvested from a banana plant obtainable by the above method of the invention, wherein the fruit flesh and/or fruit peel is characterised by a phenotype of delayed and/or reduced browning as compared to fruit flesh and/or fruit peel of a banana plant in which the level or activity of said at least one endogenous PPO1, PPO2, PPO8, PPO9, or PPO4 polyphenol oxidase is not reduced.

Yet further provided by the invention is a method of producing a banana plant characterised by a phenotype of delayed and/or reduced browning of fruit flesh and/or peel as compared to a wild-type banana plant, the method comprising: (a) providing to a banana plant cell a CRISPR-associated endonuclease or a modified CRISPR-associated endonuclease, and one or more guide RNAs, wherein said CRISPR-associated endonuclease or modified CRISPR-associated endonuclease and said one or more guide RNAs form a complex that enables the CRISPR-associated endonuclease or modified CRISPR-associated endonuclease to introduce a single-strand break or a double-strand break in at least one endogenous PPO1, PPO2, PPO8, PPO9, or PPO4 polyphenol oxidase gene: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154; and (b) identifying at least one banana plant cell that comprises a modification of said at least one endogenous PPO1, PPO2, PPO8, PPO9, or PPO4 polyphenol oxidase gene, wherein the modification is selected from the group consisting of: at least one nucleotide insertion; at least one nucleotide deletion; at least one nucleotide substitution; or any combination of the foregoing; and regenerating a banana plant from said banana plant cell, wherein said banana plant is characterised by a phenotype of delayed and/or reduced browning of fruit flesh and/or peel as compared to a wild-type banana plant.

The invention further provides a banana plant or plant part comprising in its genome at least one modified endogenous PPO1, PPO2, PPO8, PPO9, or PPO4 polyphenol oxidase gene, wherein said modification results in a reduction in, or loss of function of, the at least one endogenous PPO1, PPO2, PPO8, PPO9, or PPO4 polyphenol oxidase encoded by said modified endogenous PPO1, PPO2, PPO8, PPO9, or PPO4 polyphenol oxidase gene, wherein said modification is located in at least one endogenous PPO1, PPO2, PPO8, PPO9, or PPO4 polyphenol oxidase gene: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154.

The invention further provides banana fruit harvested from the banana plant of the invention, wherein said fruit is characterised by a phenotype of delayed and/or reduced browning as compared to fruit of a banana plant in which the level or activity of said at least one endogenous PPO1, PPO2, PPO8, PPO9, or PPO4 polyphenol oxidase is not reduced. Yet further provided by the invention is a method of obtaining a banana fruit food product, the method comprising processing the banana fruit of the invention.

Yet further provided by the invention is a DNA sequence comprising a banana PPO1, PPO2, PPO8, PPO9, or PPO4 polyphenol oxidase polynucleotide: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154.

The invention further provides a DNA construct or vector comprising a banana PPO1, PPO2, PPO8, PPO9, or PPO4 polyphenol oxidase polynucleotide: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154. Yet further provided by the invention is a plant cell transformed with the vector of the invention, optionally wherein the plant cell is a banana plant cell.

The invention further provides a polyphenol oxidase protein: encoded by SEQ ID NO: 5, SEQ ID NO: 6, SEQ ID NO: 12, SEQ ID NO: 13, or SEQ ID NO: 8, or encoded by a polynucleotide with at least 75% sequence identity to SEQ ID NO: 5, SEQ ID NO: 6, SEQ ID NO: 12, SEQ ID NO: 13, or SEQ ID NO: 8; comprising SEQ ID NO: 40, SEQ ID NO: 41, SEQ ID NO: 47, SEQ ID NO: 48, or SEQ ID NO: 43, or comprising a sequence with at least 75% sequence identity to SEQ ID NO: 40, SEQ ID NO: 41, SEQ ID NO: 47, SEQ ID NO: 48, or SEQ ID NO: 43; or encoded by SEQ ID NO: 151, SEQ ID NO: 152, SEQ ID NO: 158, SEQ ID NO: 159, or SEQ ID NO: 154, or encoded by a polynucleotide with at least 75% sequence identity to SEQ ID NO: 151, SEQ ID NO: 152, SEQ ID NO: 158, SEQ ID NO: 159, or SEQ ID NO: 154.

Yet further provided by the invention is a method of expressing a polyphenol oxidase in a plant cell, wherein the method comprises introducing into said plant cell a banana polyphenol oxidase polynucleotide: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154; operably linked to a promoter active in a plant cell.

The invention further provides a synthetic banana polyphenol oxidase guide RNA comprising a variable region selected from the group consisting of: SEQ ID NO: 32; SEQ ID NO: 33; SEQ ID NO: 34; SEQ ID NO: 35; SEQ ID NO: 57; SEQ ID NO: 58; SEQ ID NO: 38; SEQ ID NO: 39; SEQ ID NO: 62; SEQ ID NO: 76; and SEQ ID NO: 77.

The invention yet further provides a recombinant DNA construct comprising a promoter operably linked to a nucleotide sequence expressing at least one banana PPO1, PPO2, PPO8, PPO9, or PPO4 polyphenol oxidase guide RNA, wherein said guide RNA is capable of forming a complex with a CRISPR-associated endonuclease or modified CRISPR-associated endonuclease, and wherein said complex is capable of binding to and creating a double-strand or single-strand break in the at least one endogenous banana PPO1, PPO2, PPO8, or PPO9 polyphenol oxidase gene: (A) comprising a coding sequence selected from the group consisting of: SEQ ID NO: 5 or a polynucleotide with at least 75% sequence identity to SEQ ID NO: 5; SEQ ID NO: 6 or a polynucleotide with at least 75% sequence identity to SEQ ID NO: 6; SEQ ID NO: 12 or a polynucleotide with at least 75% sequence identity to SEQ ID NO: 12; SEQ ID NO: 13 or a polynucleotide with at least 75% sequence identity to SEQ ID NO: 13; and SEQ ID NO: 8 or a polynucleotide with at least 75% sequence identity to SEQ ID NO: 8; (B) encoding a polyphenol oxidase selected from the group consisting of: SEQ ID NO: 40 or a polypeptide with at least 75% sequence identity to SEQ ID NO: 40; SEQ ID NO: 41 or a polypeptide with at least 75% sequence identity to SEQ ID NO: 41; SEQ ID NO: 47 or a polypeptide with at least 75% sequence identity to SEQ ID NO: 47; SEQ ID NO: 48 or a polypeptide with at least 75% sequence identity to SEQ ID NO: 48; and SEQ ID NO: 43 or a polypeptide with at least 75% sequence identity to SEQ ID NO: 43; or (C) comprising a polynucleotide sequence selected from the group consisting of: SEQ ID NO: 151 or a polynucleotide with at least 75% sequence identity to SEQ ID NO: 151; SEQ ID NO: 152 or a polynucleotide with at least 75% sequence identity to SEQ ID NO: 152; SEQ ID NO: 158 or a polynucleotide with at least 75% sequence identity to SEQ ID NO: 158; SEQ ID NO: 159 or a polynucleotide with at least 75% sequence identity to SEQ ID NO: 159; and SEQ ID NO: 154 or a polynucleotide with at least 75% sequence identity to SEQ ID NO: 154; in the banana genome.

### BRIEF DESCRIPTION OF THE DRAWINGS AND SEQUENCE LISTING

The disclosure can be more fully understood from the following description of the accompanying drawings and Sequence Listing, which form a part of this Application.

### DRAWINGS

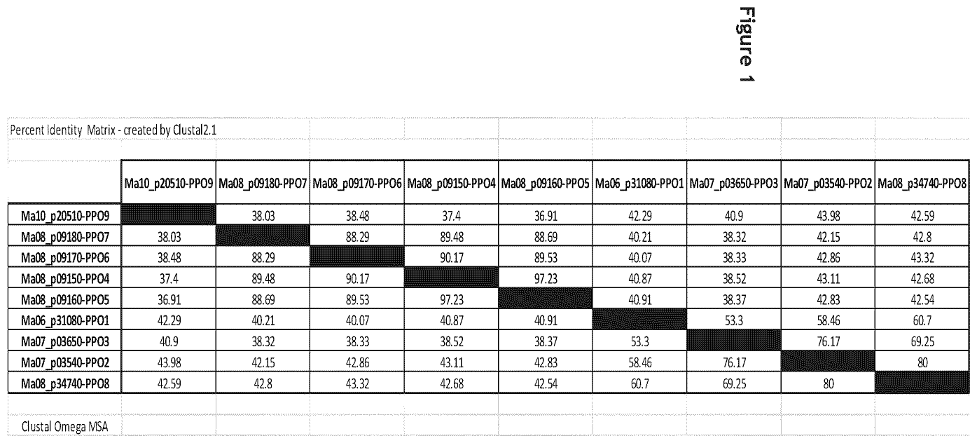

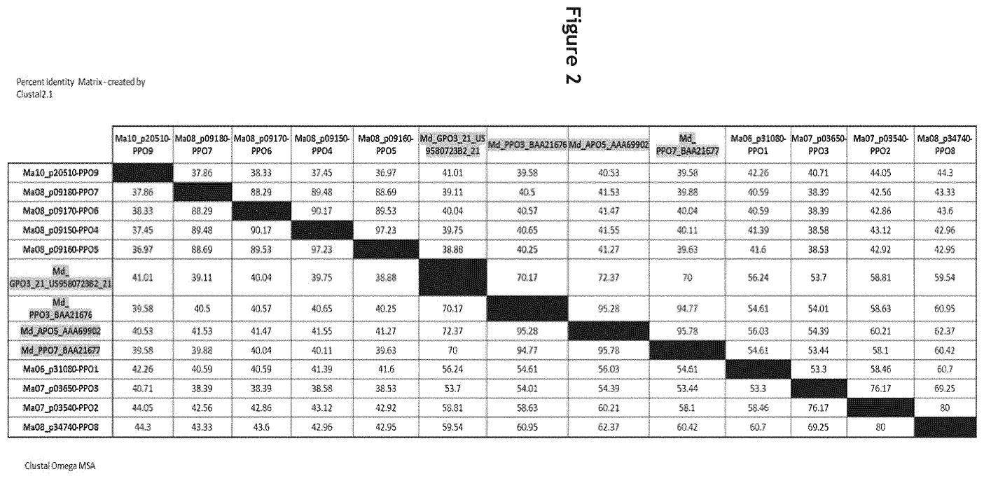

FIG. 1—Percent identity matrix of selected Musa acuminata PPO proteins. PPOs have between 35% to 97% homology to each other.

FIG. 2—Percent identity matrix of selected Musa acuminata and Malus domestica PPO proteins. PPOs have between about 39% to 97% homology to the query sequences.

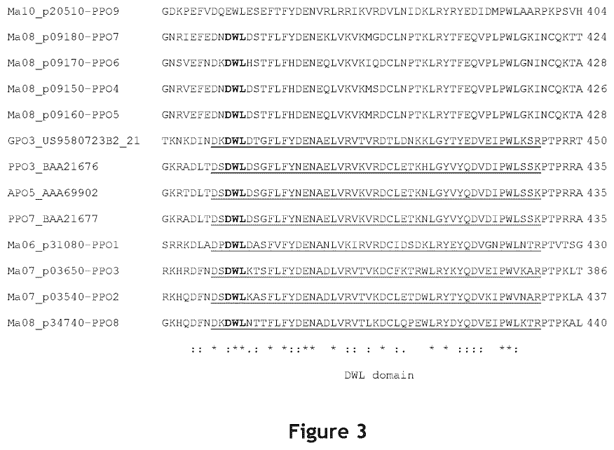

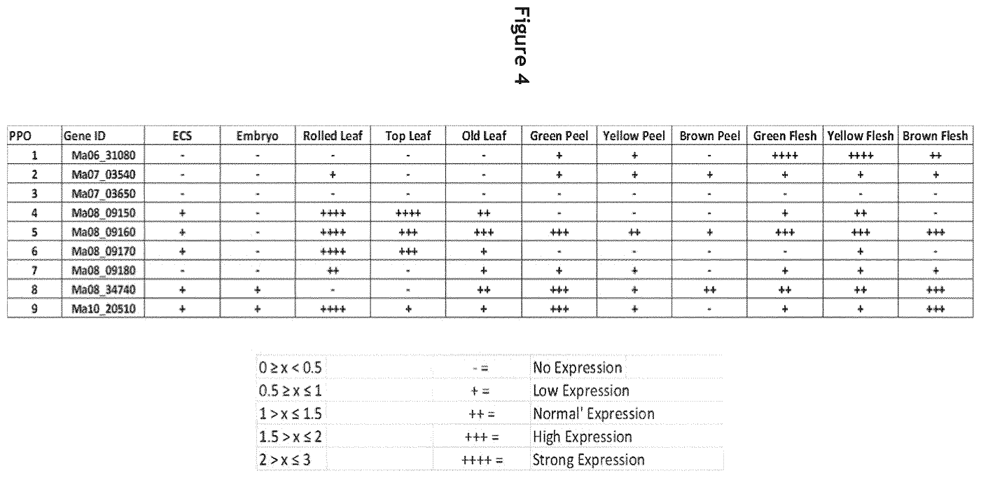

FIG. 3—Protein alignment of the DWL domain of selected PPOs targeted for gene editing. Malus domestica query sequences are shown within the box.

FIG. 4—Expression profile depicting the average expression level of each PPO in each examined tissue. The units are arbitrary, derived by visually quantifying, expressing the quantifications as numbers, and converting the numbers to a scale of − to +, ++, +++ in a linear manner.

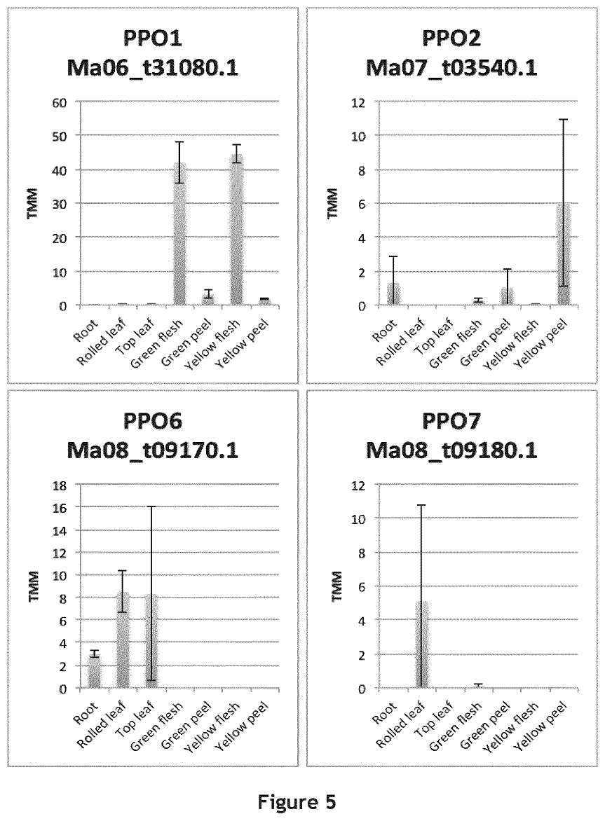

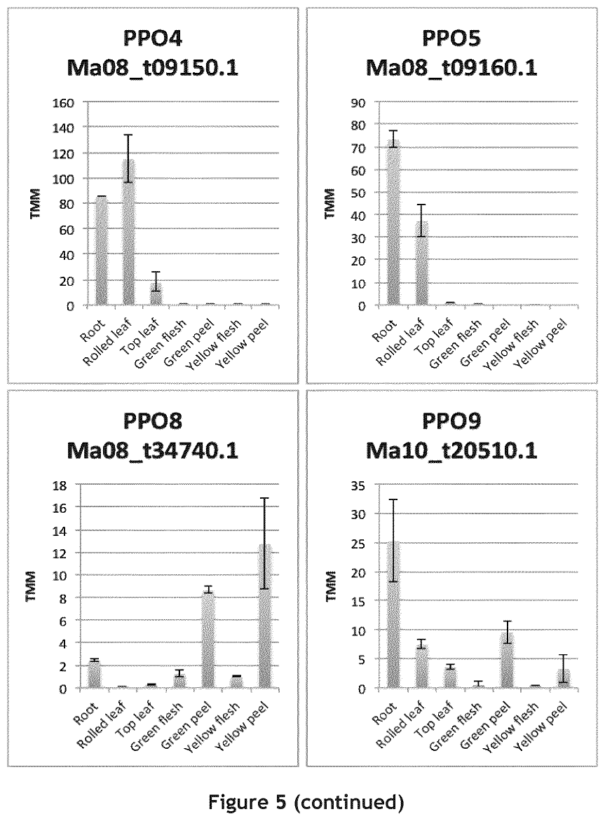

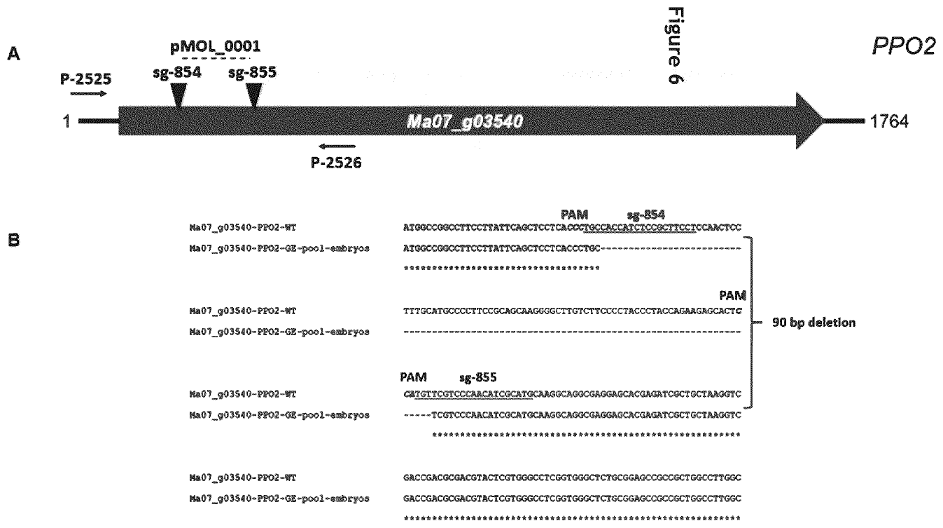

FIG. 5—Bar graphs indicating RNA-seq expression of banana PPO1, PPO2, and PPO4 to PPO9, based on comparing total RNA sequencing data from various tissues and measuring expression by TMM normalization (Trimmed Mean of M-values). Expression in banana fruit and/or flesh was seen for PPO1, PPO2, PPO8, and PPO9. TMM normalization was performed by edgeR to eliminate composition biases between and within samples. TMM normalization is a method for estimating relative RNA production levels from RNA-seq data, especially in situations where the underlying distribution of expressed transcripts between samples is markedly different. The TMM method estimates scale factors between samples. PPO3 was not detected by RNA-seq.

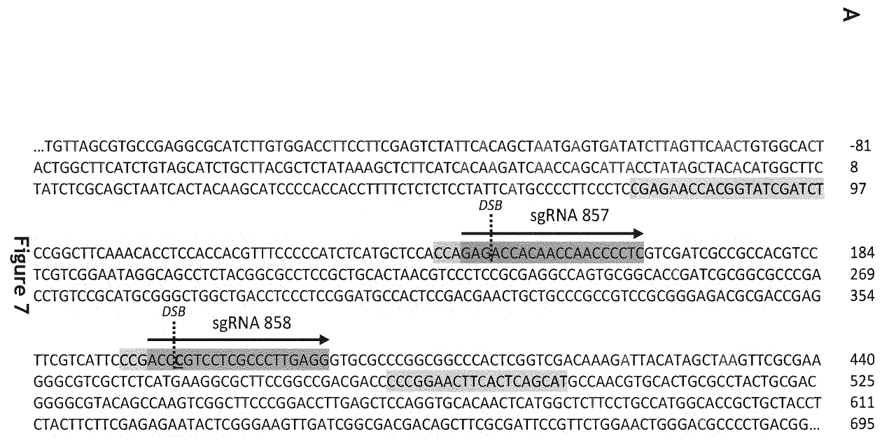

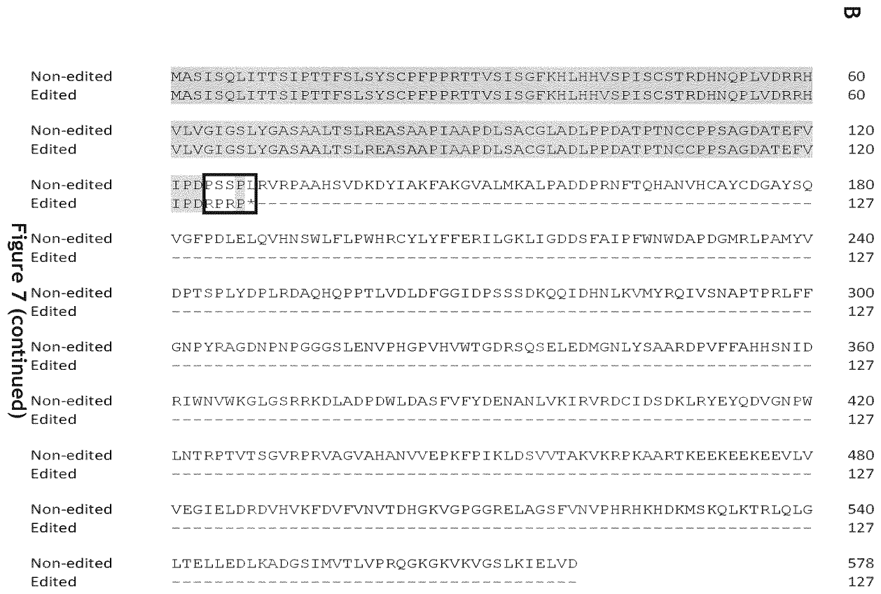

FIG. 6—Generation of an edit in PPO2, with the gene shown in FIG. 6A, and the deletion shown in FIG. 6B.

FIG. 7—(A) Partial genomic sequence from the first exon of the banana PPO1 gene. Black text indicates nucleotides from the coding sequence (nucleotides 1 to 695 from the start codon adenosine are displayed) and grey text indicates nucleotides from the upstream non-translated region (nucleotides −166 to −1 from the start codon adenosine are displayed). CRISPR/Cas9-associated DNA double-stranded break (DSB) sites are indicated with a dotted line. Two sgRNAs (857, 858) targeting the PPO1 gene are indicated in shading, and their protospacer adjacent motif (PAM) sequences are also indicated in shading. Bold typeface, and an underline further indicates the nucleotide deleted (cytosine, bp 371) in reduced browning banana plants. Shading yet further indicates primers used for PCR to amplify the PPO1 target site region for sequencing and confirmation of edits. (B) Partial alignment of PPO1 protein sequences produced from non-edited and edited PPO1 genes. The box indicates changes in protein sequence brought about by the single base pair deletion in the PPO1 gene induced by sgRNA 858-guided CRISPR/Cas9 DSB. Whereas the full-length non-edited PPO1 protein is 578 amino acids in length, the edited PPO1 protein is truncated (127 amino acids in length) due to presence of a premature stop codon (indicated with an asterisk) arising from the frameshift triggered by the single base pair deletion.

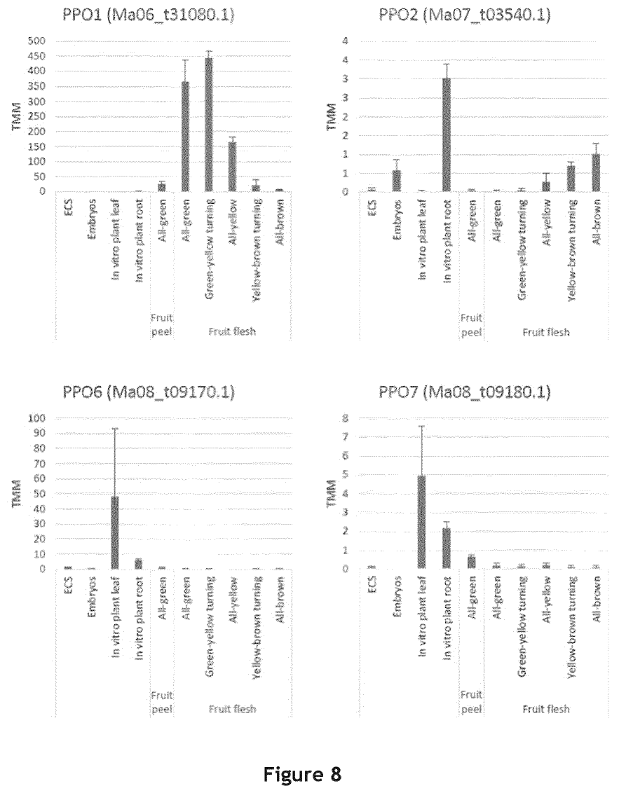

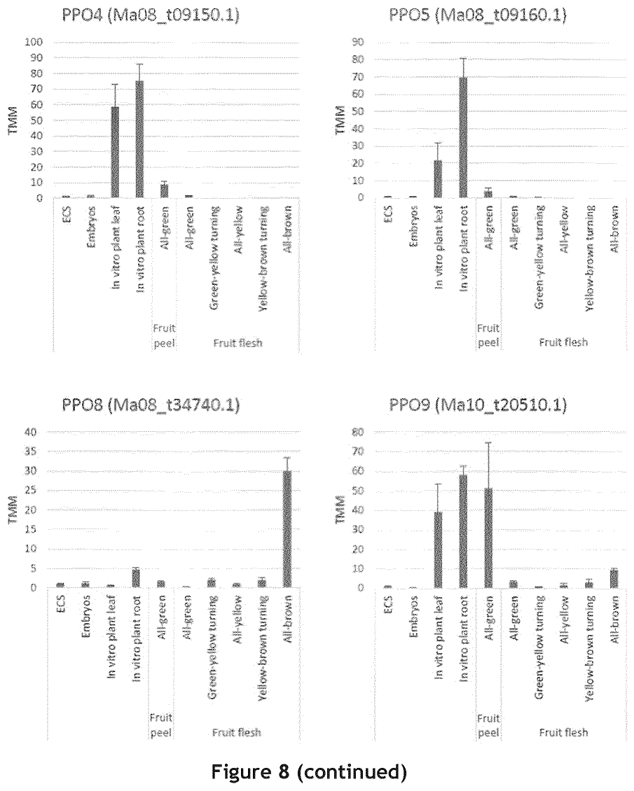

FIG. 8—RNA-seq expression analyses performed on Grande Naine bananas over the course of the natural ripening process without the application of exogenous ethylene. Peel and flesh samples were harvested from five ripening stages: all-green (mature unripe stage), green-yellow (first turning point), all-yellow (mature ripe stage), yellow-brown (second turning point), all-brown (over-ripe stage). High-quality RNA was obtained from all flesh samples and from the peel of the all-green stage. Tissue samples were also harvested from leaves and roots of in vitro Grande Naine plants and from in vitro cultures of embryos and embryogenic cells. Relative mRNA abundance was quantified as described above, using TMM normalization. PPO1, PPO4 and PPO9 account for more than 90% of PPO expression in the peel of Grande Naine bananas at the unripe green stage, whereas PPO1 is the predominant PPO gene expressed in the flesh of Grande Nanine bananas, with the exception of the over-ripe brown stage, where PPO8 is more highly expressed.

### SEQUENCES

SEQ ID NO: 1 is a PPO peptide sequence from the arctic apple.

SEQ ID NO: 2 is a PPO peptide sequence from the arctic apple.

SEQ ID NO: 3 is a PPO peptide sequence from the arctic apple.

SEQ ID NO: 4 is a PPO peptide sequence from the arctic apple.

SEQ ID NO: 5 is the PPO1 gene coding sequence from banana (Accession number Ma06_31080).

SEQ ID NO: 6 is the PPO2 gene coding sequence from banana (Accession number Ma07_03540).

SEQ ID NO: 7 is the PPO3 gene coding sequence from banana (Accession number Ma07_03650).

SEQ ID NO: 8 is the PPO4 gene coding sequence from banana (Accession number Ma08_09150).

SEQ ID NO: 9 is the PPO5 gene coding sequence from banana (Accession number Ma08_09160).

SEQ ID NO: 10 is the PPO6 gene coding sequence from banana (Accession number Ma08_09170).

SEQ ID NO: 11 is the PPO7 gene coding sequence from banana (Accession number Ma08_09180).

SEQ ID NO: 12 is the PPO8 gene coding sequence from banana (Accession number Ma08_34740).

SEQ ID NO: 13 is the PPO9 gene coding sequence from banana (Accession number Ma10_20510).

SEQ ID NO: 14 is the forward oligonucleotide used to identify the expression of mRNA-encoding PPO1.

SEQ ID NO: 15 is the reverse oligonucleotide used to identify the expression of mRNA-encoding PPO1.

SEQ ID NO: 16 is the forward oligonucleotide used to identify the expression of mRNA-encoding PPO2.

SEQ ID NO: 17 is the reverse oligonucleotide used to identify the expression of mRNA-encoding PPO2.

SEQ ID NO: 18 is the forward oligonucleotide used to identify the expression of mRNA-encoding PPO3.

SEQ ID NO: 19 is the reverse oligonucleotide used to identify the expression of mRNA-encoding PPO3.

SEQ ID NO: 20 is the forward oligonucleotide used to identify the expression of mRNA-encoding PPO4.

SEQ ID NO: 21 is the reverse oligonucleotide used to identify the expression of mRNA-encoding PPO4.

SEQ ID NO: 22 is the forward oligonucleotide used to identify the expression of mRNA-encoding PPO5.

SEQ ID NO: 23 is the reverse oligonucleotide used to identify the expression of mRNA-encoding PPO5.

SEQ ID NO: 24 is the forward oligonucleotide used to identify the expression of mRNA-encoding PPO6.

SEQ ID NO: 25 is the reverse oligonucleotide used to identify the expression of mRNA-encoding PPO6.

SEQ ID NO: 26 is the forward oligonucleotide used to identify the expression of mRNA-encoding PPO7.

SEQ ID NO: 27 is the reverse oligonucleotide used to identify the expression of mRNA-encoding PPO7.

SEQ ID NO: 28 is the forward oligonucleotide used to identify the expression of mRNA-encoding PPO8.

SEQ ID NO: 29 is the reverse oligonucleotide used to identify the expression of mRNA-encoding PPO8.

SEQ ID NO: 30 is the forward oligonucleotide used to identify the expression of mRNA-encoding PPO9.

SEQ ID NO: 31 is the reverse oligonucleotide used to identify the expression of mRNA-encoding PPO9.

SEQ ID NO: 32 is the variable sequence of the PPO1 sgRNA1 sg2019.

SEQ ID NO: 33 is the variable sequence of the PPO1 sgRNA2 sg858.

SEQ ID NO: 34 is the variable sequence of the PPO2 sgRNA1 sg854.

SEQ ID NO: 35 is the variable sequence of the PPO2 sgRNA2 sg855.

SEQ ID NO: 36 is the variable sequence of the PPO3 sgRNA1 sg1435.

SEQ ID NO: 37 is the variable sequence of the PPO3 sgRNA2 sg1436.

SEQ ID NO: 38 is the variable sequence of the PPO9 sgRNA1 sg850.

SEQ ID NO: 39 is the variable sequence of the PPO9 sgRNA2 sg851.

SEQ ID NO: 40 is the PPO1 polypeptide sequence from banana (Accession number Ma06_31080).

SEQ ID NO: 41 is the PPO2 polypeptide sequence from banana (Accession number Ma07_03540).

SEQ ID NO: 42 is the PPO3 polypeptide sequence from banana (Accession number Ma07_03650).

SEQ ID NO: 43 is the PPO4 polypeptide sequence from banana (Accession number Ma08_09150).

SEQ ID NO: 44 is the PPO5 polypeptide sequence from banana (Accession number Ma08_09160).

SEQ ID NO: 45 is the PPO6 polypeptide sequence from banana (Accession number Ma08_09170).

SEQ ID NO: 46 is the PPO7 polypeptide sequence from banana (Accession number Ma08_09180).

SEQ ID NO: 47 is the PPO8 polypeptide sequence from banana (Accession number Ma08_34740).

SEQ ID NO: 48 is the PPO9 polypeptide sequence from banana (Accession number Ma10_20510).

SEQ ID NO: 49 is the variable sequence of PPO1 sgRNA sg2019 including PAM site.

SEQ ID NO: 50 is the variable sequence of PPO1 sgRNA sg858 including PAM site.

SEQ ID NO: 51 is the variable sequence of PPO2 sgRNA sg854 including PAM site.

SEQ ID NO: 52 is the variable sequence of PPO2 sgRNA sg855 including PAM site.

SEQ ID NO: 53 is the variable sequence of PPO3 sgRNA sg1435 including PAM site.

SEQ ID NO: 54 is the variable sequence of PPO3 sgRNA sg1436 including PAM site.

SEQ ID NO: 55 is the variable sequence of PPO9 sgRNA sg850 including PAM site.

SEQ ID NO: 56 is the variable sequence of PPO9 sgRNA sg851 including PAM site.

SEQ ID NO: 57 is the variable sequence of the PPO8 sgRNA1 sg852.

SEQ ID NO: 58 is the variable sequence of the PPO8 sgRNA2 sg853.

SEQ ID NO: 59 is the variable sequence of an alternative PPO1 sgRNA.

SEQ ID NO: 60 is the variable sequence of an alternative PPO1 sgRNA.

SEQ ID NO: 61 is the variable sequence of an alternative PPO1 sgRNA.

SEQ ID NO: 62 is the variable sequence of an alternative PPO1 sgRNA (sg857).

SEQ ID NO: 63 is the variable sequence of an alternative PPO1 sgRNA.

SEQ ID NO: 64 is the variable sequence of an alternative PPO2 sgRNA.

SEQ ID NO: 65 is the variable sequence of an alternative PPO2 sgRNA.

SEQ ID NO: 66 is the variable sequence of an alternative PPO2 sgRNA.

SEQ ID NO: 67 is the variable sequence of an alternative PPO2 sgRNA.

SEQ ID NO: 68 is the variable sequence of an alternative PPO2 sgRNA.

SEQ ID NO: 69 is the variable sequence of an alternative PPO2 sgRNA.

SEQ ID NO: 70 is the variable sequence of an alternative PPO2 sgRNA.

SEQ ID NO: 71 is the variable sequence of an alternative PPO3 sgRNA.

SEQ ID NO: 72 is the variable sequence of an alternative PPO3 sgRNA.

SEQ ID NO: 73 is the variable sequence of an alternative PPO3 sgRNA.

SEQ ID NO: 74 is the variable sequence of an alternative PPO3 sgRNA.

SEQ ID NO: 75 is the variable sequence of an alternative PPO3 sgRNA.

SEQ ID NO: 76 is the variable sequence of an alternative PPO4 sgRNA.

SEQ ID NO: 77 is the variable sequence of an alternative PPO4 sgRNA.

SEQ ID NO: 78 is the variable sequence of an alternative PPO5 sgRNA.

SEQ ID NO: 79 is the variable sequence of an alternative PPO5 sgRNA.

SEQ ID NO: 80 is the variable sequence of an alternative PPO6 sgRNA.

SEQ ID NO: 81 is the variable sequence of an alternative PPO6 sgRNA.

SEQ ID NO: 82 is the variable sequence of an alternative PPO7 sgRNA.

SEQ ID NO: 83 is the variable sequence of an alternative PPO7 sgRNA.

SEQ ID NO: 84 is the variable sequence of an alternative PPO7 sgRNA.

SEQ ID NO: 85 is the variable sequence of an alternative PPO7 sgRNA.

SEQ ID NO: 86 is the variable sequence of an alternative PPO7 sgRNA.

SEQ ID NO: 87 is the variable sequence of an alternative PPO7 sgRNA.

SEQ ID NO: 88 is the variable sequence of an alternative PPO7 sgRNA.

SEQ ID NO: 89 is the variable sequence of an alternative PPO7 sgRNA.

SEQ ID NO: 90 is the variable sequence of an alternative PPO8 sgRNA.

SEQ ID NO: 91 is the variable sequence of an alternative PPO8 sgRNA.

SEQ ID NO: 92 is the variable sequence of an alternative PPO8 sgRNA.

SEQ ID NO: 93 is the variable sequence of an alternative PPO8 sgRNA.

SEQ ID NO: 94 is the variable sequence of an alternative PPO9 sgRNA.

SEQ ID NO: 95 is the variable sequence of an alternative PPO9 sgRNA.

SEQ ID NO: 96 is the variable sequence of an alternative PPO9 sgRNA.

SEQ ID NO: 97 is the variable sequence of an alternative PPO9 sgRNA.

SEQ ID NO: 98 is the scaffold used with the variable sequences of the sgRNA.

SEQ ID NO: 99 is the variable sequence of PPO8 sgRNA sg852 including PAM site.

SEQ ID NO: 100 is the variable sequence of PPO8 sgRNA sg853 including PAM site.

SEQ ID NO: 101 is the variable sequence of an alternative PPO1 sgRNA including PAM site.

SEQ ID NO: 102 is the variable sequence of an alternative PPO1 sgRNA including PAM site.

SEQ ID NO: 103 is the variable sequence of an alternative PPO1 sgRNA including PAM site.

SEQ ID NO: 104 is the variable sequence of an alternative PPO2 sgRNA including PAM site.

SEQ ID NO: 105 is the variable sequence of an alternative PPO2 sgRNA including PAM site.

SEQ ID NO: 106 is the variable sequence of an alternative PPO2 sgRNA including

PAM site.

SEQ ID NO: 107 is the variable sequence of an alternative PPO3 sgRNA including PAM site.

SEQ ID NO: 108 is the variable sequence of an alternative PPO3 sgRNA including

PAM site.

SEQ ID NO: 109 is the variable sequence of a PPO7 sgRNA including PAM site.

SEQ ID NO: 110 is the variable sequence of a PPO7 sgRNA including PAM site.

SEQ ID NO: 111 is the variable sequence of a PPO7 sgRNA including PAM site.

SEQ ID NO: 112 is the variable sequence of a PPO7 sgRNA including PAM site.

SEQ ID NO: 113 is the variable sequence of a PPO7 sgRNA including PAM site.

SEQ ID NO: 114 is the variable sequence of a PPO7 sgRNA including PAM site.

SEQ ID NO: 115 is the variable sequence of an alternative PPO8 sgRNA including PAM site.

SEQ ID NO: 116 is the variable sequence of an alternative PPO8 sgRNA including PAM site.

SEQ ID NO: 117 is the variable sequence of an alternative PPO8 sgRNA including PAM site.

SEQ ID NO: 118 is the variable sequence of an alternative PPO8 sgRNA including PAM site.

SEQ ID NO: 119 is the variable sequence of an alternative PPO9 sgRNA including PAM site.

SEQ ID NO: 120 is the variable sequence of an alternative PPO9 sgRNA including PAM site.

SEQ ID NO: 121 is the variable sequence of an alternative PPO9 sgRNA including

PAM site.

SEQ ID NO: 122 is the variable sequence of an alternative PPO9 sgRNA including PAM site.

SEQ ID NO: 123 is a forward primer to detect a gene editing event in PPO1. SEQ ID NO: 124 is a reverse primer to detect a gene editing event in PPO1.

SEQ ID NO: 125 is a forward primer to detect a gene editing event in PPO2.

SEQ ID NO: 126 is a reverse primer to detect a gene editing event in PPO2.

SEQ ID NO: 127 is a forward primer to detect a gene editing event in PPO3.

SEQ ID NO: 128 is a reverse primer to detect a gene editing event in PPO3.

SEQ ID NO: 129 is a forward primer to detect a gene editing event in PPO8.

SEQ ID NO: 130 is a reverse primer to detect a gene editing event in PPO8.

SEQ ID NO: 131 is a forward primer to detect a gene editing event in PPO9.

SEQ ID NO: 132 is a reverse primer to detect a gene editing event in PPO9.

SEQ ID NO: 133 is primer 1684 used to confirm absence of Cas9.

SEQ ID NO: 134 is primer 1685 used to confirm absence of Cas9.

SEQ ID NO: 135 is primer 1686 used to confirm absence of Cas9.

SEQ ID NO: 136 is primer 1687 used to confirm absence of Cas9.

SEQ ID NO: 137 is primer 1563 used to confirm absence of backbone.

SEQ ID NO: 138 is primer 1564 used to confirm absence of backbone.

SEQ ID NO: 139 is primer 1565 used to confirm absence of backbone.

SEQ ID NO: 140 is primer 1566 used to confirm absence of backbone.

SEQ ID NO: 141 is primer 1567 used to confirm absence of backbone.

SEQ ID NO: 142 is primer 1568 used to confirm absence of backbone.

SEQ ID NO: 143 is primer 1569 used to confirm absence of backbone.

SEQ ID NO: 144 is primer 1570 used to confirm absence of backbone.

SEQ ID NO: 145 is primer 1571 used to confirm absence of backbone.

SEQ ID NO: 146 is primer 1572 used to confirm absence of backbone.

SEQ ID NO: 147 is primer 1573 used to confirm absence of backbone.

SEQ ID NO: 148 is primer 1574 used to confirm absence of backbone.

SEQ ID NO: 149 is primer 1575 used to confirm absence of backbone.

SEQ ID NO: 150 is primer 1576 used to confirm absence of backbone.

SEQ ID NO: 151 is the PPO1 gene sequence from banana (Accession number Ma06_31080).

SEQ ID NO: 152 is the PPO2 gene sequence from banana (Accession number Ma07_03540).

SEQ ID NO: 153 is the PPO3 gene sequence from banana (Accession number Ma07_03650).

SEQ ID NO: 154 is the PPO4 gene sequence from banana (Accession number Ma08_09150).

SEQ ID NO: 155 is the PPO5 gene sequence from banana (Accession number Ma08_09160).

SEQ ID NO: 156 is the PPO6 gene sequence from banana (Accession number Ma08_09170).

SEQ ID NO: 157 is the PPO7 gene sequence from banana (Accession number Ma08_09180).

SEQ ID NO: 158 is the PPO8 gene sequence from banana (Accession number Ma08_34740).

SEQ ID NO: 159 is the PPO9 gene sequence from banana (Accession number Ma10_20510).

SEQ ID NO: 160 is the wheat TaU6 promoter.

SEQ ID NO: 161 is Ma10_p20510-PPO9 from FIG. 3.

SEQ ID NO: 162 is Ma08_p09180-PPO7 from FIG. 3.

SEQ ID NO: 163 is Ma08_p09170-PPO6 from FIG. 3.

SEQ ID NO: 164 is Ma08_p09150-PPO4 from FIG. 3.

SEQ ID NO: 165 is Ma08_p09160-PPO5 from FIG. 3.

SEQ ID NO: 166 is GPO3_21_US9580723B2_21 from FIG. 3.

SEQ ID NO: 167 is PPO3_BAA21676 from FIG. 3.

SEQ ID NO: 168 is APO5_AAA69902 from FIG. 3.

SEQ ID NO: 169 is PPO7_BAA21677 from FIG. 3.

SEQ ID NO: 170 is Ma06_p31080-PPO1 from FIG. 3.

SEQ ID NO: 171 is Ma07_p03650-PPO3 from FIG. 3.

SEQ ID NO: 172 is Ma07_p03540-PPO2 from FIG. 3.

SEQ ID NO: 173 is Ma08_p34740-PPO8 from FIG. 3.

SEQ ID NO: 174 is Ma07_g03540-PPO2-WT from FIG. 6.

SEQ ID NO: 175 is Ma07_g03450-PPO2-GE-pool-embryos.

SEQ ID NO: 176 is the variable sequence of PPO1 sgRNA sg857 including PAM site.

SEQ ID NO: 177 is a PPO1 mutant protein sequence.

SEQ ID NO: 178 is a PPO1 mutant coding sequence.

SEQ ID NO: 179 is a PPO1 mutant gene sequence.

SEQ ID NO:180 to SEQ ID NO:225 are the sgRNAs provided in Table 8, which do not include PAM sites.

### DETAILED DESCRIPTION OF THE INVENTION

The present invention is based in part on the finding that there are nine Polyphenol Oxidase (PPO) genes that exist in banana (which the inventors termed PPO1-PPO9), most of which have not been previously identified as PPO genes. The present invention is further based in part on the finding that some of the identified PPOs, and in particular Polyphenol Oxidase 1 (PPO1), Polyphenol Oxidase 2 (PPO2), Polyphenol Oxidase 8 (PPO8), Polyphenol Oxidase 9 (PPO9), and Polyphenol Oxidase 4 (PPO4) are expressed in the flesh and/or peel of banana fruit, but characterised by lower or absent expression in other tissues, and in particular embryonic tissue or Embryonic Cell Suspension (ECS). Without wishing to be bound by theory or mechanism, reducing the level or activity of PPO1, PPO2, PPO8, PPO9, and/or PPO4 in a banana plant, according to some embodiments of the invention, will delay or prevent the browning of the flesh and/or peel of banana fruit without causing unwanted effects in other plant parts. According to some embodiments, the invention is directed to various methods of reducing the level or activity of PPOs, in particular PPO1, PPO2, PPO8, PPO9, and/or PPO4, in a banana plant or banana plant cell in order to delay or prevent banana fruit browning. Products of such methods are also provided by the invention.

In particular, the invention provides a method of reducing the level or activity of at least one endogenous polyphenol oxidase encoded by a polyphenol oxidase gene or polynucleotide in a banana plant or banana plant cell. The invention further provides a method for reducing and/or delaying browning of at least one of banana fruit and banana peel. According to some embodiments, provided herein is a method for reducing and/or delaying browning of at least one of the flesh and peel of a banana fruit, the method comprising reducing or inducing a loss of function of at least one endogenous PPO of a banana plant cell or a banana plant which give rise to the banana fruit (e.g. by reducing or inducing a loss of the expression of a gene encoding the at least one endogenous PPO). Each possibility represents a separate embodiment of the present invention. According to some embodiments, the endogenous PPO is PPO1, PPO2, PPO8, PPO9, and/or PPO4. According to some embodiments, the PPO is selected from the group consisting of: PPO1, PPO2, PPO3, PPO4, PPO5, PPO6, PPO7, PPO8, and PPO9.

As used herein, the term “reducing the level or activity of” one or more endogenous polyphenol oxidases means that: (a) the delayed or reduced browning phenotype conferred by the one or more endogenous polyphenol oxidases in the banana plant or banana plant cell or the function of the one or more endogenous polyphenol oxidases in the banana plant or banana plant cell is reduced as compared to in “wild-type” banana plants or plant cells; and/or (b) the expression level of a gene encoding the one or more endogenous polyphenol oxidases in the banana plant or banana plant cell is reduced as compared to in “wild-type” banana plants or plant cells. Expression level can be mRNA or protein. Such reduction may be by at least 50%, 60%, 70%, 80%, 90%, or 100%, preferably. According to some embodiments, reduction is complete loss of function. In this context, “wild-type” means the same genetic background and a comparable developmental stage. In some embodiments, the method of the invention results in a reduction in function of the at least one endogenous PPO1, PPO2, PPO8, PPO9, or PPO4 polyphenol oxidase in said banana plant or banana plant cell. In other embodiments, the method results in a loss of function of the at least one endogenous PPO1, PPO2, PPO8, PPO9, or PPO4 polyphenol oxidase in said banana plant or banana plant cell. In some embodiments, the method of the invention results in a reduction in function of the at least one endogenous polyphenol oxidase in said banana plant or banana plant cell, wherein the at least one polyphenol oxidase is selected from the group consisting of: PPO1, PPO2, PPO3, PPO4, PPO5, PPO6, PPO7, PPO8, PPO9, and combinations thereof. In other embodiments, the method results in a loss of function of the at least one endogenous polyphenol oxidase in said banana plant or banana plant cell, wherein the at least one polyphenol oxidase is selected from the group consisting of: PPO1, PPO2, PPO3, PPO4, PPO5, PPO6, PPO7, PPO8, PPO9, and combinations thereof. As used herein, the term “endogenous” means native to the genome of the banana plant or banana plant cell, and at the native position within the genome.

As used herein, the term “polyphenol oxidase” or “PPO” refers to an enzyme belonging to a group of enzymes found throughout the plant and animal kingdoms, including in most fruits and vegetables, such as banana. Correspondingly, the term “polyphenol oxidase gene” or “PPO gene” as used herein refers to gene encoding a polyphenol oxidase or PPO. PPOs catalyse enzymatic browning, including when tissues are damaged from bruising or compression. Exposure to oxygen when fruit is sliced or pureed also leads to enzymatic browning by PPOs. PPOs are known to accept monophenols and/or o-diphenols as substrates, and work by catalyzing the o-hydroxylation of monophenol molecules in which the benzene ring contains a single hydroxyl substituent to o-diphenols (phenol molecules containing two hydroxyl substituents at the 1, 2 positions, with no carbon between). The enzymes can also further catalyse the oxidation of o-diphenols to produce o-quinones. PPOs catalyse the rapid polymerization of o-quinones to produce black, brown or red pigments (polyphenols) that cause fruit browning.

While many plants contain a few PPO members, the PPO family in banana has not been characterised to date. The inventors identified nine endogenous PPOs in banana (Musa acuminata DH-Pahang), which are termed “PPO1”, “PPO2”, “PPO3”, “PPO4”, “PPO5”, “PPO6”, “PPO7”, “PPO8”, and “PPO9”. As described herein, these PPOs were identified through several iterations of complex phylogenetic analyses on genomes of various species including: Coffea canephora, Phoenix dactylifera, Musa acuminata banksia, Musa acuminata Calcutta, Musa acuminata DH-Pahang, Musa balbisiana, Musa itinerans, Arabidopsis thaliana, Glycine max, Malus domestica (apple), Capsicum annuum, Nicotiana benthamiana, Solanum lycopersicum, and Oryza sativa.

In some embodiments, the level or activity of endogenous PPO1 is reduced. In some embodiments, the activity of endogenous PPO2 is reduced. In some embodiments, the level or activity of endogenous PPO3 is reduced. In some embodiments, the level or activity of endogenous PPO4 is reduced. In some embodiments, the level or activity of endogenous PPO5 is reduced. In some embodiments, the level or activity of endogenous PPO6 is reduced. In some embodiments, the level or activity of endogenous PPO7 is reduced. In some embodiments, the level or activity of endogenous PPO8 is reduced. In some embodiments, the level or activity of endogenous PPO9 is reduced. In some embodiments, the level or activity of two or more of endogenous PPOs selected from the group consisting of PPO1, PPO2, PPO3, PPO4, PPO5, PPO6, PPO7, PPO8, and PPO9 is reduced. In particular embodiments, the level or activity of endogenous PPO1 and PPO2 is reduced. In any of these embodiments, the term “reducing the level or activity of” one or more endogenous polyphenol oxidases selected from the group consisting of PPO1, PPO2, PPO3, PPO4, PPO5, PPO6, PPO7, PPO8, and PPO9 means that the phenotype conferred by these one or more endogenous polyphenol oxidases selected from the group consisting of PPO1, PPO2, PPO3, PPO4, PPO5, PPO6, PPO7, PPO8, and PPO9 in the banana plant or banana plant cell or the function of the one or more endogenous polyphenol oxidases selected from the group consisting of PPO1, PPO2, PPO3, PPO4, PPO5, PPO6, PPO7, PPO8, and PPO9 in the banana plant or banana plant cell is reduced as compared to in wild-type banana plants or plant cells, or lost completely.

An endogenous PPO gene in any of the embodiments herein can be described as having “sequence identity” or homology to a polynucleotide sequence. The amount of homology or sequence identity can vary, and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the endogenous PPO gene sequence or polynucleotide sequence.

These ranges include every integer within the range, for example, the range of 1-20 bp includes 1, 2, 3, 4, 5, 6, 7, 8, 9, 10, 11, 12, 13, 14, 15, 16, 17, 18, 19 and 20 bp. The amount of homology or sequence identity can also be described by percent sequence identity over the full aligned length of the two genes or two polynucleotides, which includes percent sequence identity of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Sufficient homology includes any combination of polynucleotide length, global percent sequence identity, and optionally conserved regions of contiguous nucleotides or local percent sequence identity. For example, sufficient homology can be described as a region of 75-150 bp having at least 80% sequence identity to a region of the endogenous PPO gene sequence or polynucleotide sequence. Sufficient homology can also be described by the predicted ability of two genes or polynucleotides to specifically hybridize under high stringency conditions. In this regard, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

In particular embodiments of the invention, the PPO1 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5; the PPO2 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6; the PPO3 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7; the PPO4 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8; the PPO5 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9; the PPO6 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10; the PPO7 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11; the PPO8 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12; or the PPO9 gene refers to a polynucleotide sequence which comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13. In particular embodiments of the invention, the PPO1, PPO2, PPO8, or PPO9 polyphenol oxidase gene of the invention comprises a coding sequence that is selected from the group consisting of: SEQ ID NO: 5 (PPO1) or a polynucleotide with at least 75% sequence identity to SEQ ID NO: 5; SEQ ID NO: 6 (PPO2) or a polynucleotide with at least 75% sequence identity to SEQ ID NO: 6; SEQ ID NO: 12 (PPO8) or a polynucleotide with at least 75% sequence identity to SEQ ID NO: 12; and SEQ ID NO: 13 (PPO9) or a polynucleotide with at least 75% sequence identity to SEQ ID NO: 13.

In particular embodiments of the invention, the PPO1 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40; the PPO2 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41; the PPO3 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42; the PPO4 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43; the PPO5 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44; the PPO6 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45; the PPO7 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46; the PPO8 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47; or the PPO9 gene refers to a polynucleotide sequence which encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In particular embodiments of the invention, the PPO1, PPO2, PPO8, or PPO9 polyphenol oxidase gene of the invention encodes a polyphenol oxidase selected from the group consisting of: SEQ ID NO: 40 (PPO1) or a polypeptide with at least 75% sequence identity to SEQ ID NO: 40; SEQ ID NO: 41 (PPO2) or a polypeptide with at least 75% sequence identity to SEQ ID NO: 41; SEQ ID NO: 47 (PPO8) or a polypeptide with at least 75% sequence identity to SEQ ID NO: 47; and SEQ ID NO: 48 (PPO9) or a polypeptide with at least 75% sequence identity to SEQ ID NO: 48.

In particular embodiments of the invention, the PPO1 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151;

the PPO2 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152; the PPO3 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153; the PPO4 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154;

the PPO5 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155; the PPO6 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156; the PPO7 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157;

the PPO8 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158; or the PPO9 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159. In particular embodiments of the invention, the PPO1, PPO2, PPO8, or PPO9 polyphenol oxidase gene of the invention comprises a polynucleotide sequence selected from the group consisting of: SEQ ID NO: 151 (PPO1) or a polynucleotide with at least 75% sequence identity to SEQ ID NO: 151;

SEQ ID NO: 152 (PPO2) or a polynucleotide with at least 75% sequence identity to SEQ ID NO: 152; SEQ ID NO: 158 (PPO8) or a polynucleotide with at least 75% sequence identity to SEQ ID NO: 158; and SEQ ID NO: 159 (PPO9) or a polynucleotide with at least 75% sequence identity to SEQ ID NO: 159.

When reference is made herein to particular sequences, these may also encompass sequences that substantially correspond to its complementary sequence as including minor sequence variations, resulting from, e.g. sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

Any Sequence Identification Number (SEQ ID NO) disclosed herein can refer to either a DNA sequence or a RNA sequence, depending on the context where that SEQ ID NO is mentioned, even if that SEQ ID NO is expressed only in a DNA sequence format or a RNA sequence format. For example, a given SEQ ID NO: is expressed in a DNA sequence format (e.g. reciting T for thymine), but it can refer to either a DNA sequence that corresponds to a given nucleic acid sequence, or the RNA sequence of an RNA molecule nucleic acid sequence. Similarly, though some sequences are expressed in a RNA sequence format (e.g. reciting U for uracil), depending on the actual type of molecule being described, it can refer to either the sequence of a RNA molecule comprising a double-stranded RNA (dsRNA), or the sequence of a DNA molecule that corresponds to the RNA sequence shown. In any event, both DNA and RNA molecules having the sequences disclosed with any substitutes are envisaged.

When percentage sequence identity is used in reference to proteins, residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g. according to the algorithm of Henikoff S and Henikoff J G., Proc. Natl. Acad. Sci. U.S.A. 1992, 89(22): 10915-9. Identity can be determined using any homology comparison software, including for example, the “BlastN” software of the National Center of Biotechnology Information (NCBI) such as by using default parameters. In some embodiments, the identity is a global identity, i.e. an identity over an entire nucleic acid sequence and not over portions thereof.

As used herein, the term “plant” refers to whole plants, grafted plants, ancestors and progeny of the plants, plant organs, plant tissues, and “plant parts”. “Plant parts”, as used herein, include differentiated and undifferentiated tissues including, but not limited to roots (including tubers), rootstocks, stems, scions, shoots, fruits, leaves, pollens, seeds, tumor tissue, and various forms of cells and culture (e.g. single cells, protoplasts, embryos, embryonic cells, and callus tissue). The plant tissue may be in plant or in a plant organ, tissue or cell culture.

In particular embodiments, the plant part is a fruit. Fruit comprises tissues such as fruit flesh and fruit peel. In other embodiments, the plant part is a seed. The term “seed” as used herein refers to a unit of reproduction of a flowering plant capable of developing into another such plant. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

A “transgenic plant” includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant.

A “heterologous” polynucleotide, as used herein, comprises a sequence that originates from a foreign species.

“Transgenic” can include any cell, cell line, callus, tissue, plant part or plant, the genotype of which has been altered by the presence of heterologous nucleic acid including those transgenics initially so altered as well as those created by sexual crosses or asexual propagation from the initial transgenic. In some plants, the heterologous polynucleotide which is introduced into the plant genome can be removed through breeding. This process is not possible in banana, as described hereinabove.

According to some embodiments, the banana cells, banana plants, or banana plant parts described herein are non-transgenic. According to some embodiments, the methods disclosed herein result in a banana cell, banana plant or banana plant cell which is non-transgenic.

The alterations of the genome (chromosomal or extra-chromosomal) by conventional plant breeding methods, by the genome editing procedure described herein (that does not result in an insertion of a foreign polynucleotide), or by naturally occurring events such as random cross-fertilization, non-recombinant viral infection, non-recombinant bacterial transformation, non-recombinant transposition, or spontaneous mutation are not intended to be regarded as transgenic.

In certain embodiments, a fertile plant is a plant that produces viable male and female gametes and is self-fertile. Such a self-fertile plant can produce a progeny plant without the contribution from any other plant of a gamete and the genetic material contained therein. Other embodiments can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. Male-sterile and female-sterile plants can be female-fertile and male-fertile, respectively. A male fertile (but female sterile) plant can produce viable progeny when crossed with a female fertile plant and that a female fertile (but male sterile) plant can produce viable progeny when crossed with a male fertile plant.

As used herein, the term “banana plant” refers to a plant of the genus Musa, including plantains. These include Musa acuminata (e.g. Musa acuminata banksia, Musa acuminata Calcutta, and Musa acuminata DH-Pahang), Musa balbisiana, Musa itinerans, and autotriploid Musa acuminata ‘Cavendish’ and ‘Gros Michel’. According to a specific embodiment, banana is autotriploid Musa acuminata ‘Cavendish’. Cultivated bananas are infertile autotriploids (AAA) derived from the progenitor species Musa acuminata (genome AA). Additionally, plantains (AAB or ABB) are infertile interspecific allotriploids derived from the hybridisation of Musa acuminata (AA) and Musa balbisiana (genome BB). The triploid nature of cultivated banana and plantain prevents them from producing viable seeds, whereas wild species are diploid and can produce viable seeds. In particular embodiments, the banana plant is triploid. Other ploidies are contemplated, including diploid and tetraploid.

In some embodiments, the “banana plant” is of a banana breeding line, such as an elite line or purebred line, or a banana variety or breeding germplasm. The term “breeding line”, as used herein, refers to a line of a cultivated banana having commercially valuable or agronomically desirable characteristics, as opposed to wild varieties or landraces. The term includes reference to an “elite breeding line” or “elite line”, which represents an essentially homozygous, usually inbred, line of plants used to produce commercial F1 hybrids. An “elite breeding line” is obtained by breeding and selection for superior agronomic performance comprising a multitude of agronomically desirable traits. An “elite plant” is any plant from an elite line. Superior agronomic performance refers to a desired combination of agronomically desirable traits as defined herein, wherein it is desirable that the majority, preferably all of, the agronomically desirable traits are improved in the elite breeding line as compared to a non-elite breeding line. Elite breeding lines are essentially homozygous and are preferably inbred lines. The term “elite line”, as used herein, refers to any line that has resulted from breeding and selection for superior agronomic performance.

The terms “cultivar” and “variety” are used interchangeable herein and denote a plant with has deliberately been developed by breeding, e.g. crossing and selection, for the purpose of being commercialized, e.g. used by farmers and growers, to produce agricultural products for own consumption or for commercialization. The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and/or backcrossing.

As used herein, the term “banana plant cell” is a cell of a banana plant. Banana plant cells include cells from seeds, suspension cultures, embryos, meristematic regions, callus tissue, leaves, roots, shoots, gametophytes, sporophytes, pollen, microspores, embryogenic cells, somatic cells, and protoplasts. Protoplasts can be derived from any plant tissue, such as, but not limited to, roots, leaves, embryonic cell suspension, callus, or seedling tissue. According to some embodiments, a banana plant cell is a cell of an Embryonic Cell Suspension (ECS).

In some embodiments, method of the invention results in delayed browning of fruit flesh and/or fruit peel of said banana plant as compared to fruit flesh and/or fruit peel of a banana plant in which the level or activity of said at least one endogenous polyphenol oxidase (selected from the group consisting of: PPO1, PPO2, PPO3, PPO4, PPO5, PPO6, PPO7, PPO8, and PPO9) is not reduced; and/or reduced browning of fruit flesh and/or fruit peel of said banana plant as compared to fruit flesh and/or fruit peel of a banana plant in which the level or activity of said at least one endogenous polyphenol oxidase (selected from the group consisting of: PPO1, PPO2, PPO3, PPO4, PPO5, PPO6, PPO7, PPO8, and PPO9) is not reduced. In some embodiments, the method of the invention results in delayed and/or reduced browning of flesh and/or peel of a banana fruit derived from a banana plant in which the level or activity of at least one endogenous polyphenol oxidase selected from PPO1, PPO2, PPO8, PPO9, and PPO4 is reduced or lost. In some embodiments, the method of the invention results in delayed and/or reduced browning of flesh and/or peel of a banana fruit derived from a banana plant in which the expression of at least one endogenous polyphenol oxidase genes selected from PPO1, PPO2, PPO8, PPO9, and PPO4 is reduced or lost.

In this context, “browning” of banana fruit flesh and/or banana fruit peel can be measured by visual inspection and other methods known in the art.

In some embodiments, “browning” is measured based on peel colour. In such embodiments, a browning index is constructed such that a particular colour of the peel of wild-type banana fruits (e.g. green, partly green, yellow, brown etc) can be correlated to the number of days that have passed from flower appearance. Using such an index, a visual assessment of the colour of the peel at a certain day can provide an indication of delayed browning (e.g. if the colour of a wild-type banana is brown a certain number of days from flower appearance, but a banana genetically edited in a PPO gene is yellow after the same number of days from flower appearance) (see “Dole Retail Banana Ripening Guide”, https://www.dolenz.co.nz/uploads/media/59236082048a9/banana-trade-section-web.pdf, and Gooding et al., “Molecular cloning and characterisation of banana fruit polyphenol oxidase”, Planta, September 2001; 123(5): 748-57). This is based on the fact that fruits mature in about 60 to 90 days after flower appearance. Banana fruits are collected as early as 40 days up until they turn very brown (stage 10). Stage 10 is expected to be reached around 90 days after flower appearance. The fruit colour is visually assessed at all 10 stages based on the description of colour defined, for example, in the “Dole Retail Banana Ripening Guide”. Stage 1 is when all fingers of the bunch have green peel, while at stage 7, all fingers of the bunch have yellow-flecked with brown peel, and at stage 10, all fingers of the bunch have dark-brown peel. To standardise the measure of colour, colorimetric coordinates are taken with a Minolta Chroma Meter CR 400 or a Minolta CR-300 Chroma Meter with DP-301 Data Processor. The measuring head of the CR-300 uses diffuse illumination/0° viewing geometry (specular component included) to provide measurements of a wide variety of surfaces which correlate well with colour, as seen under diffuse lighting conditions, as is described in Bruno Bonnet, C., Hubert, O., Mbeguie-A-Mbeguie, D. et al., Effect of physiological harvest stages on the composition of bioactive compounds in Cavendish bananas. J. Zhejiang Univ. Sci. B 14, 270-278 (2013). This allows measuring reflected colour at each stage of fruit development (1 to 10), and these data are to correlate peel colour to fruit ripening and browning.

In some embodiments, “browning” is measured based on a banana browning guide that makes use of a visual assessment of sliced and pureed banana pulp (flesh) over time (from 0 to 180 hours). In such embodiments, 3 fingers are collected from banana bunches representing stages 3 to 10 (see above), and the peel is gently (to avoid mechanical damage that may lead to browning) washed for 5 minutes in 0.2% sodium hypochlorite. Next, banana puree is prepared from each individual banana finger after peeling, cutting into pieces, and homogenisation in an electrical blender or food processor. The resulting puree is poured on Petri dishes, and images are captured at 0, 15, 30, 60, and 120 minutes and later at 24, 48, and 72 hours. In addition, banana slices are cut and placed on Petri dishes, and images are captured at 0, 12, 24, 36, 48, and 72 hours for banana bunches at colour stage 3 and 4. For banana fingers at stages 5 up to 10, images of banana slices are captured every 8 hours from 0 to 180 hours (˜22 time points) (see Chi, M., Bhagwat, B., Lane, W. D. et al. Reduced polyphenol oxidase gene expression and enzymatic browning in potato (Solanum tuberosum L.) with artificial microRNAs. BMC Plant Biol 14, 62 (2014). https://doi.org/10.1186/1471-2229-14-62, and Escalante-Minakata, P., Ibarra-Junquera, V., Ornelas-Paz, J.d. et al., Comparative study of the banana pulp browning process of ‘Giant Dwarf’ and FHIA-23 during fruit ripening based on image analysis and the polyphenol oxidase and peroxidase biochemical properties. 3 Biotech 8, 30 (2018)). Images are processed to correlate image colour to browning. Colour is expected to vary somewhere from freshly cut banana slices or banana puree colour (yellow/off-white) to brown bananas (dark brown).

In some embodiments, “browning” is measured based on an assessment of pulp firmness and peel hardness. In such embodiments, pulp firmness and peel hardness are measured with a TA-XT2 penetrometer as described in Bruno Bonnet, C., Hubert, O., Mbeguie-A-Mbeguie, D. et al., Effect of physiological harvest stages on the composition of bioactive compounds in Cavendish bananas. J. Zhejiang Univ. Sci. B 14, 270-278 (2013). Three banana fingers are taken from bunches representing stages 3 to 10 (as established based on peel colour; see above). Using a cylindrical 4.9 mm metal borer, the clean, fresh, unpeeled fruits are penetrated at a constant speed (2 mm/s) to a depth of 10 mm. The maximum force applied to break the peel represents the peel hardness, and the slope of the force/time curve represents the fruit firmness.

In some embodiments, “browning” is measured based on a correlation of peel colour (and firmness) with flesh colour/texture. Such embodiments make use of a catalogue of colour (visual and colorimetric measurements), peel hardness, and pulp firmness vs banana maturation stages and browning of peel and flesh relative to time in wild type plants. This creates the standard by which a reduction of browning can be assessed in banana peel and flesh.

In some embodiments, “browning” is measured based on the amount of melanin formed in the banana tissue being analysed within a set time frame. Such a quantification of browning can be performed in simple biochemical assays (Michael L. Sullivan et al., Cloning and Characterization of Red Clover Polyphenol Oxidase cDNAs and Expression of Active Protein in Escherichia coli and Transgenic Alfalfa. Plant Physiology October 2004, 136 (2) 3234-3244; DOI: 10.1104/pp. 104.047449; Matthew A.

Escobar et al., Characterization of Polyphenol Oxidase from Walnut. Journal of the American Society for Horticultural Science November 2008, Volume 133: Issue 6, pages 852-858; DOI: 10.21273/JASHS.133.6.852). In these enzymatic browning assays, a change in the absorbance of an exogenous PPO substrate is measured after mixing with banana tissue lysate, and both the amount of product formed (melanin) and the rate of the browning reaction are proportional to PPO activity.

In some embodiments, for example when considering ripening-associated browning, “delayed” browning means that the onset of browning of fruit flesh and/or fruit peel is delayed by at least one day, at least two days, at least three days, at least four days, at least five days, at least six days, at least a week, at least two weeks, at least three weeks, or at least a month, as compared to fruit flesh and/or fruit peel of a banana plant in which the level or activity of the at least one endogenous polyphenol oxidase (selected from the group consisting of: PPO1, PPO2, PPO3, PPO4, PPO5, PPO6, PPO7, PPO8, and PPO9) is not reduced. According to certain embodiments, “delayed” refers to delay of at least 3 days. Browning may be measured as outlined above.

In some embodiments, for example when considering bruising-associated or slicing-associated browning, “delayed” browning means that the onset of browning of fruit flesh and/or fruit peel is delayed by at least one hour, at least two hours, at least three hours, at least four hours, at least five hours, at least six hours, at least seven hours, at least eight hours, at least nine hours, or at least ten hours, as compared to fruit flesh and/or fruit peel of a banana plant in which the level or activity of the at least one endogenous polyphenol oxidase (selected from the group consisting of: PPO1, PPO2, PPO3, PPO4, PPO5, PPO6, PPO7, PPO8, and PPO9) is not reduced. Browning may be measured as outlined above.

In some embodiments, “reduced” browning means that it takes twice as long, three times as long, four times as long, five times as long, six times as long, seven times as long, eight times as long, nine times as long, or ten times as long, for browning of fruit flesh and/or fruit peel to occur as compared to fruit flesh and/or fruit peel of a banana plant in which the level or activity of the at least one endogenous polyphenol oxidase (selected from the group consisting of: PPO1, PPO2, PPO3, PPO4, PPO5, PPO6, PPO7, PPO8, and PPO9) is not reduced. Browning may be measured as outlined above.

In some embodiments, “reduced” browning means that less melanin is formed at defined time point. For example, there may be at least a 10%, 20%, 30%, 40%, 50%, three-fold, 5-fold, 10-fold or larger reduction in melanin in fruit flesh and/or fruit peel to occur as compared to fruit flesh and/or fruit peel of a control banana plant in which the level or activity of the at least one endogenous polyphenol oxidase (selected from the group consisting of: PPO1, PPO2, PPO3, PPO4, PPO5, PPO6, PPO7, PPO8, and PPO9) is not reduced. Melanin measurements can be taken as outlined above, at any time point. For example, when considering ripening-associated browning, melanin can be measured at the point at which the control banana has reached stage 7, 8, 9 or 10 as defined in Example 1 below and the banana of the invention is of the same age in terms of number of days since flowering. For example, two or more times less melanin may be present in the banana of the invention at stage 7, 8, 9 or 10, three or more times less melanin may be present in the banana of the invention at stage 7, 8, 9 or 10, or five or more times less melanin may be present in the banana of the invention at stage 7, 8, 9 or 10.

In some embodiments, the method of the invention comprises providing to a banana plant cell or to a part of the banana plant a silencing RNA targeting a transcript of at least one endogenous polyphenol oxidase gene selected from the group consisting of: PPO1, PPO2, PPO3, PPO4, PPO5, PPO6, PPO7, PPO8, and PPO9. In some embodiments, a transcript of the endogenous PPO1 gene is targeted. In some embodiments, a transcript of the endogenous PPO2 gene is targeted. In some embodiments, a transcript of the endogenous PPO3 gene is targeted. In some embodiments, a transcript of the endogenous PPO4 gene is targeted. In some embodiments, a transcript of the endogenous PPO5 gene is targeted. In some embodiments, a transcript of the endogenous PPO6 gene is targeted. In some embodiments, a transcript of the endogenous PPO7 gene is targeted. In some embodiments, a transcript of the endogenous PPO8 gene is targeted. In some embodiments, a transcript of the endogenous PPO9 gene is targeted. In some embodiments, transcripts of two or more of endogenous PPO1, PPO2, PPO3, PPO4, PPO5, PPO6, PPO7, PPO8, and PPO9 are targeted. In particular embodiments, transcripts of endogenous PPO1 and PPO2 are targeted.

The silencing RNA can be provided to the banana plant cell or to a part of the banana plant by a method selected from the group consisting of: introducing exogenous silencing RNA; introducing a sequence expressing the silencing RNA into the cell (i.e. creating a transgene); or transiently editing an endogenous gene encoding an existing non-coding RNA (such as a silencing RNA) to redirect (and possibly activate) its silencing specificity towards a target gene encoding said at least one polyphenol oxidase, optionally PPO1, PPO2, PPO8, PPO9; and/or PPO4 (see WO 2019/058255, which is incorporated herein by reference). Potential silencing RNAs that could be introduced, expressed, or redirected include, but are not limited to, a small interfering RNA (siRNA), a short hairpin RNA (shRNA), a microRNA (miRNA), a Piwi-interacting RNA (piRNA), a phased small interfering RNA (phasiRNA), a trans-acting siRNA (tasiRNA), a transfer RNA (tRNA), a small nuclear RNA (snRNA), and autonomous and non-autonomous transposable RNA.

In other embodiments, the method of the invention comprises introducing a modification into at least one endogenous polyphenol oxidase gene selected from the group consisting of: PPO1, PPO2, PPO3, PPO4, PPO5, PPO6, PPO7, PPO8, and PPO9. In some embodiments, a modification is introduced into the endogenous PPO1 gene. In some embodiments, a modification is introduced into the endogenous PPO2 gene. In some embodiments, a modification is introduced into the endogenous PPO3 gene. In some embodiments, a modification is introduced into the endogenous PPO4 gene. In some embodiments, a modification is introduced into the endogenous PPO5 gene. In some embodiments, a modification is introduced into the endogenous PPO6 gene. In some embodiments, a modification is introduced into the endogenous PPO7 gene. In some embodiments, a modification is introduced into the endogenous PPO8 gene. In some embodiments, a modification is introduced into the endogenous PPO9 gene. In some embodiments, a modification is introduced into two or more of endogenous PPO1, PPO2, PPO3, PPO4, PPO5, PPO6, PPO7, PPO8, and PPO9. In particular embodiments, a modification is introduced into endogenous PPO1 and PPO2. Thus, according to some embodiments, provided herein is a method of reducing the level or activity of at least one endogenous polyphenol oxidase encoded by a polyphenol oxidase gene selected from the group consisting of: PPO1, PPO2, PPO3, PPO4, PPO5, PPO6, PPO7, PPO8, and PPO9; wherein the method comprises introducing a modification into the at least one endogenous polyphenol oxidase gene selected from the group consisting of: PPO1, PPO2, PPO3, PPO4, PPO5, PPO6, PPO7, PPO8, and PPO9.

In this regard, “introducing a modification” means introducing at least one mutation into at least one allele of the one or more endogenous polyphenol oxidase genes. According to some embodiments, a mutation is introduced into one allele of the one or more endogenous polyphenol oxidase genes, whereas the two other alleles do not include the mutation. According to some embodiments, a mutation is introduced into two alleles of the one or more endogenous polyphenol oxidase genes, whereas the third allele does not include the mutation. According to some embodiments, a mutation is introduced into each allele of the one or more endogenous polyphenol oxidase genes. In any of the embodiments, the mutation can be in a homozygous form or in a heterozygous form. A “modification”, as used herein, can mean at least one nucleotide insertion, at least one nucleotide deletion, an insertion-deletion (indel), an inversion, at least one nucleotide substitution, or any combination of the foregoing, provided that it results in a reduction in the level or activity of at least one endogenous PPO. The modification can result in a frameshift, a missense mutation, loss-of-function mutation, or a nonsense mutation, in the one or more corresponding endogenous polyphenol oxidase genes, such that the one or more endogenous polyphenol oxidase genes are not expressed or expressed at a reduced level. In any embodiment, the size of the modification can be smaller than 1 kb or even smaller than 0.1 kb. In some embodiments, the loss-of-function mutation is in the 5′ region of the respective PPO gene so as to inhibit the production of any expression product (for example in exon 1).

However, the loss-of-function mutation may be in any part of the respective PPO gene, such as, but not limited to, in regulatory elements of the gene (for example its promoter).

In some embodiments, the modification is introduced into the one or more polyphenol oxidase genes by an endonuclease provided to said banana plant cell and capable of targeting said at least one polyphenol oxidase gene, such as at least one of the PPO1, PPO2, PPO8, PPO9, or PPO4 polyphenol oxidase genes.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site. Endonucleases allow for precision genetic engineering of eukaryotic genomes, such as plant genomes.

In some embodiments, the endonuclease is selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease. According to some embodiments, the endonuclease is a CRISPR-associated endonuclease, optionally wherein the CRISPR-associated endonuclease is Cas9. Each possibility represents a separate embodiment of the present invention. In some embodiments, the endonuclease is selected from the group consisting of: a meganuclease, a zinc finger nuclease (ZFN), a transcription-activator like effector nuclease (TALEN), and a homing endonuclease.

According to some embodiments, provided herein is a method of reducing the level or activity of at least one endogenous polyphenol oxidase (such as the PPO1, PPO2, PPO8, PPO9, or PPO4 polyphenol oxidase encoded by a PPO1, PPO2, PPO8, PPO9, or PPO4 polyphenol oxidase gene) in a banana plant or banana plant cell; wherein reducing the level or activity of the at least one endogenous polyphenol oxidase comprises introducing a modification into the polyphenol oxidase gene encoding said at least one polyphenol oxidase; wherein said modification is introduced by providing an endonuclease which is capable of targeting said at least one polyphenol oxidase gene to said banana plant or banana plant cell; and wherein the endonuclease is a CRISPR-associated endonuclease or a modified CRISPR-associated endonuclease. According to some embodiments, the endonuclease is provided to the banana cell together with at least one “targeting molecule” enabling the endonuclease to specifically target a polyphenol oxidase gene of choice. According to some embodiments, the targeting molecule is a guide RNA which enables a CRISPR-associated endonuclease or a modified CRISPR-associated endonuclease to cleave a sequence of choice within a polyphenol oxidase gene.

As used herein, “meganucleases” are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, but the number of such naturally occurring meganucleases is limited. In order to overcome this challenge, mutagenesis and high throughput screening methods have been used to create meganuclease variants that recognize unique sequences. For example, various meganucleases have been fused to create hybrid enzymes that recognize a new sequence. Alternatively, DNA-interacting amino acids of the meganuclease can be altered to design sequence specific meganucleases (see e.g., U.S. Pat. No. 8,021,867). Meganucleases can be designed using the methods described in e.g. Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514. Alternatively, meganucleases with site-specific cutting characteristics can be obtained using commercially available technologies e.g. Precision Biosciences' Directed Nuclease Editor™ genome editing technology.

“Zinc finger nucleases” (or “ZFNs”) and “transcription-activator like effector nucleases” (or “TALENs”), as used herein, have proven to be effective at producing targeted double-stranded breaks (see Christian M, Cermak T, Doyle E L, et al. Targeting DNA double-strand breaks with TAL effector nucleases. Genetics. 2010; 186(2):757-761. doi:10.1534/genetics. 110.120717). In essence, ZFNs and TALENs restriction endonuclease technology utilizes a non-specific DNA cutting enzyme linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally, FokI has the advantage of requiring dimerization to have nuclease activity, which means that the specificity increases, because each nuclease partner recognizes a unique DNA sequence. To enhance this effect, FokI nucleases have been engineered that can only function as heterodimers and have increased catalytic activity. Such nucleases avoid the possibility of unwanted homodimer activity and increase specificity of the double-stranded break. Thus, to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-strand break. Repair of these double-stranded breaks through the “non-homologous end-joining” (or “NHEJ”) pathway often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have been successfully generated in cell culture by using two pairs of nucleases simultaneously (Carlson D F, Fahrenkrug S C, Hackett P B. Targeting DNA With Fingers and TALENs. Mol Ther Nucleic Acids. 2012; 1(1):e3. Published 2012 Jan. 24. doi: 10.1038/mtna.2011.5). In addition, when a fragment of DNA with homology to the targeted region is introduced in conjunction with the nuclease pair, the double-strand break or double-stranded break can be repaired via homologous recombination (HR) to generate specific modifications (Urnov, F., Miller, J., Lee, Y. et al. Highly efficient endogenous human gene correction designed zinc-finger nucleases. Nature 435, 646-651 using (2005). https://doi.org/10.1038/nature03556). Although the nuclease portions of both ZFNs and TALENs have similar properties, the difference between these engineered nucleases is in their DNA recognition peptide. ZFNs rely on Cys2-His2 zinc fingers, and TALENs on TALEs. Both of these DNA recognizing peptide domains have the characteristic that they are naturally found in combinations in their proteins. Cys2-His2 Zinc fingers are typically found in repeats that are 3 bp apart, and are found in diverse combinations in a variety of nucleic acid interacting proteins. TALEs, on the other hand, are found in repeats with a one-to-one recognition ratio between the amino acids and the recognized nucleotide pairs. Because both zinc fingers and TALEs happen in repeated patterns, different combinations can be tried to create a wide variety of sequence specificities.

Approaches for making site-specific zinc finger endonucleases include, for example, modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from, for example, Sangamo Biosciences™ (Richmond, CA). Methods for designing and obtaining TALENs are described in Reyon et al. Nature Biotechnology (2012) 30(5): 460-465; Miller et al. Nature Biotechnology (2011) 29: 143-148; Cermak et al. Nucleic Acids Research (2011) 39(12): e82, and Zhang et al. Nature Biotechnology (2011) 29(2): 149-153. A recently developed web-based program named “Mojo Hand” was introduced by Mayo Clinic for designing TAL and TALEN constructs for genome editing applications (can be accessed through www.talendesign.org).

As used herein, “homing endonucleases” are double-stranded DNases that have large, asymmetric recognition sites (12 to 40 base pairs (bp)) and coding sequences that are usually embedded in either introns or inteins (Belfort, M. and Roberts, R. J. (1997) Nucleic Acids Research, 25, 3379-3388). Introns are spliced out of precursor RNAs, while inteins are spliced out of precursor proteins (Dujon, B. et al. (1989) Gene, 82, 115-118; Perler, F. B. et al. (1994) Nucleic Acids Research, 22, 1125-1127). Homing endonucleases are named using conventions similar to those of restriction endonucleases with intron-encoded endonucleases containing the prefix, “I-” and intein endonucleases containing the prefix, “PI-” (Belfort, M. and Roberts, R. J. (1997) Nucleic Acids Research, 25, 3379-3388; Roberts, R. J. et al. (2003) Nucleic Acids Research, 31, 1805-1812). Homing endonuclease recognition sites are rare. For example, an 18-base pair (bp) recognition sequence will occur only once in every 7×10¹⁰ base pairs of random sequence. However, unlike restriction endonucleases, homing endonucleases tolerate some sequence degeneracy within their recognition sequence (Gimble, F. S. and Wang, J. (1996) Journal of Molecular Biology, 263, 163-180; Argast, M. G. et al. (1998) Journal of Molecular Biology, 280, 345-353). That is, single base changes do not abolish cleavage but reduce its efficiency to variable extents. As a result, their observed sequence specificity is typically in the range of 10 to 12 base pairs.

In some embodiments, the method of the invention comprises providing to a banana plant cell a CRISPR-associated endonuclease or a modified CRISPR-associated endonuclease. In some embodiments, the method of the invention comprises providing to a banana plant cell a CRISPR-associated endonuclease or a modified CRISPR-associated endonuclease, and one or more guide RNAs specific to at least one polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at the at least one endogenous polyphenol oxidase gene. In some of these embodiments, said CRISPR-associated endonuclease is a Cas9 endonuclease. In some of these embodiments, the at least one endogenous polyphenol oxidase gene or polynucleotide is selected from the group consisting of: PPO1, PPO2, PPO3, PPO4, PPO5, PPO6, PPO7, PPO8, and PPO9. In some embodiments, the at least one endogenous polyphenol oxidase gene is a PPO1 gene. In some embodiments, the at least one endogenous polyphenol oxidase gene is a PPO2 gene. In some embodiments, the at least one endogenous polyphenol oxidase gene is a PPO3 gene. In some embodiments, the at least one endogenous polyphenol oxidase gene is a PPO4 gene. In some embodiments, the at least one endogenous polyphenol oxidase gene is a PPO5 gene. In some embodiments, the at least one endogenous polyphenol oxidase gene is a PPO6 gene. In some embodiments, the at least one endogenous polyphenol oxidase gene is a PPO7 gene. In some embodiments, the at least one endogenous polyphenol oxidase gene is a PPO8 gene. In some embodiments, the at least one endogenous polyphenol oxidase gene is a PPO9 gene. In some embodiments, the endogenous polyphenol oxidase genes are two or more of PPO1, PPO2, PPO3, PPO4, PPO5, PPO6, PPO7, PPO8, and PPO9 genes. In some embodiments, the endogenous polyphenol oxidase genes are PPO1 and PPO2 genes.

In particular embodiments of the method, the PPO1 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5; the PPO2 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6; the PPO3 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7; the PPO4 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8; the PPO5 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9; the PPO6 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10; the PPO7 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11; the PPO8 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12; or the PPO9 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13. In particular embodiments of the method, the PPO1, PPO2, PPO8, PPO9, or PPO4 polyphenol oxidase gene comprises a coding sequence that is selected from the group consisting of SEQ ID NO: 5 (PPO1) or a polynucleotide with at least 75% sequence identity to SEQ ID NO: 5; SEQ ID NO: 6 (PPO2) or a polynucleotide with at least 75% sequence identity to SEQ ID NO: 6; SEQ ID NO: 12 (PPO8) or a polynucleotide with at least 75% sequence identity to SEQ ID NO: 12; SEQ ID NO: 13 (PPO9) or a polynucleotide with at least 75% sequence identity to SEQ ID NO: 13; and SEQ ID NO: 8 (PPO4) or a polynucleotide with at least 75% sequence identity to SEQ ID NO: 8.

In particular embodiments of the method, the PPO1 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40; the PPO2 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41; the PPO3 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42; the PPO4 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43; the PPO5 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44; the PPO6 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45; the PPO7 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46; the PPO8 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47; or the PPO9 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In particular embodiments of the method, the PPO1, PPO2, PPO8, PPO9, or PPO4 polyphenol oxidase gene encodes a polyphenol oxidase selected from the group consisting of: SEQ ID NO: 40 (PPO1) or a polypeptide with at least 75% sequence identity to SEQ ID NO: 40; SEQ ID NO: 41 (PPO2) or a polypeptide with at least 75% sequence identity to SEQ ID NO: 41; SEQ ID NO: 47 (PPO8) or a polypeptide with at least 75% sequence identity to SEQ ID NO: 47; SEQ ID NO: 48 (PPO9) or a polypeptide with at least 75% sequence identity to SEQ ID NO: 48; and SEQ ID NO: 43 (PPO4) or a polypeptide with at least 75% sequence identity to SEQ ID NO: 43.

In particular embodiments of the method, the PPO1 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151; the PPO2 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152; the PPO3 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153; the PPO4 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154; the PPO5 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155; the PPO6 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156; the PPO7 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157; the PPO8 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158; or the PPO9 gene refers to a polynucleotide sequence which comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159. In particular embodiments of the method, the PPO1, PPO2, PPO8, PPO9, or

PPO4 polyphenol oxidase gene comprises a polynucleotide sequence selected from the group consisting of: SEQ ID NO: 151 (PPO1) or a polynucleotide with at least 75% sequence identity to SEQ ID NO: 151; SEQ ID NO: 152 (PPO2) or a polynucleotide with at least 75% sequence identity to SEQ ID NO: 152; SEQ ID NO: 158 (PPO8) or a polynucleotide with at least 75% sequence identity to SEQ ID NO: 158; SEQ ID NO: 159 (PPO9) or a polynucleotide with at least 75% sequence identity to SEQ ID NO: 159; and SEQ ID NO: 154 (PPO4) or a polynucleotide with at least 75% sequence identity to SEQ ID NO: 154.

As used herein, a “CRISPR-associated endonuclease” (or “Cas”) refers to an endonuclease having an RNA-guided polynucleotide-editing activity and is one of the components of the CRISPR/Cas system for genome editing, which uses at least one additional component, a “guide RNA” (gRNA). In some embodiments of the invention, the “CRISPR-associated endonuclease” is a “Cas9 endonuclease” (or “Cas9”). According to some embodiments, the “CRISPR-associated endonuclease” may be any Cas9 known in the art, such as, but not limited to, SpCas9, SaCas9, FnCas9, NmCas9, St1Cas9, BlatCas9 (Shota Nakade, Takashi Yamamoto & Tetsushi Sakuma (2017), Cas9, Cpf1 and C2c1/2/3-What's next?, Bioengineered, 8:3, 265-273, and references therein). In other embodiments, the “CRISPR-associated endonuclease” may be Cpf1, such as, but not limited to, AsCpf1 or LbCpf1 (Shota Nakade, Takashi Yamamoto & Tetsushi Sakuma (2017), Cas9, Cpf1 and C2c1/2/3-What's next?, Bioengineered, 8:3, 265-273, and references therein).

The terms “guide RNA” or “gRNA” as used herein may be used interchangeably and refer to a polynucleotide which facilitates the specific targeting of a CRISPR-associated endonuclease or a modified CRISPR-associated endonuclease to a target sequence such as a genomic or episomal sequence in a cell. According to some embodiments, gRNAs can be chimeric/uni-molecular (comprising a single RNA molecule, also referred to as single guide RNA or sgRNA) or modular (comprising more than one separate RNA molecule, typically a crRNA and tracrRNA which may be linked, for example by duplexing). According to some embodiments, a gRNA is an sgRNA.

The sgRNA is an RNA molecule which includes both the tracrRNA and crRNA (and a connecting loop). The sgRNA comprises a nucleotide sequence encoding the target homologous sequence (crRNA) and the endogenous bacterial RNA that links the crRNA to the Cas nuclease (tracrRNA) in a single chimeric transcript. This region of the crRNA, known as the variable region, confers the cutting specificity of the associated endonuclease and is typically 20 nucleotides in length, but can be between about 17 to 20 nucleotides in length. The gRNA/Cas complex is recruited to the target sequence by the base-pairing between the gRNA sequence and the complement genomic DNA. For successful binding of Cas, the genomic target sequence must also contain the correct Protospacer Adjacent Motif (PAM) sequence immediately following the target sequence. The binding of the gRNA/Cas complex localizes the Cas to the genomic target sequence so that the Cas can cut both strands of the DNA causing a double-strand or double-stranded break. Just as with ZFNs and TALENs, the double-stranded breaks produced by CRISPR/Cas can be repaired by HR (homologous recombination) or NHEJ (non-homologous end-joining), and are susceptible to specific sequence modification during DNA repair. The Cas nuclease has two functional domains: RuvC and HNH, each cutting a different DNA strand. When both of these domains are active, the Cas causes double strand breaks in the genomic DNA. A significant advantage of CRISPR/Cas is the high efficiency of this system coupled with the ability to easily create synthetic gRNAs. This creates a system that can be readily modified to target different genomic sites and/or to target different modifications at the same site. Additionally, protocols have been established which enable simultaneous targeting of multiple genes. The majority of cells carrying the mutation present biallelic mutations in the targeted genes. However, apparent flexibility in the base-pairing interactions between the gRNA sequence and the genomic DNA target sequence allows imperfect matches to the target sequence to be cut by Cas.

Modified versions of the Cas enzyme containing a single inactive catalytic domain, either RuvC- or HNH-, are called ‘nickases’. With only one active nuclease domain, the Cas nickase cuts only one strand of the target DNA, creating a single-strand break or “nick”. A single-strand break or single-stranded break, or nick, is mostly repaired by single strand break repair mechanism involving proteins such as but not only, PARP (sensor) and XRCCI/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas nickase are treated as a double-strand break, in what is often referred to as a “double nick” CRISPR system. A double-nick which is basically non-parallel DSB can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas nickase to create a double-nick by designing two gRNAs with target sequences in close proximity and on opposite strands of the genomic DNA would decrease off-target effect as either gRNA alone will result in nicks that are not likely to change the genomic DNA, even though these events are not impossible.

As used herein, a “modified CRISPR-associated endonuclease” (or “modified Cas”) refers to a Cas in which the catalytic domain has been altered and/or which are fused to additional domain. According to some embodiments, a “modified Cas” refers to a Cas which contains inactive catalytic domains (dead Cas, or dCas) and has no nuclease activity while still being able to bind to DNA based on gRNA specificity.

According to some embodiments, a “modified Cas” refers to a Cas which has a nickase activity (“nCas9”), thus inducing a single strand break. In some embodiments, the modified CRISPR-associated endonuclease is a “modified Cas9 endonuclease”, possibly a catalytically inactive Cas9 (or “dCas9”) or a nickase Cas9 (“nCas9”). The dCas can be utilized as a platform for DNA transcriptional regulators to activate or repress gene expression by fusing the inactive enzyme to known regulatory domains. For example, the binding of dCas alone to a target sequence in genomic DNA can interfere with gene transcription. There are a number of publically available tools available to help choose and/or design target sequences as well as lists of bioinformatically determined unique gRNAs for different genes in different species such as the Feng Zhang lab's Target Finder, the Michael Boutros lab's Target Finder “E-CRISP”, the RGEN Tools: “Cas-OFFinder”, the CasFinder: Flexible algorithm for identifying specific Cas9 targets in genomes and the CRISPR Optimal Target Finder.

In the context of the invention, modified Cas, such as dCas or nCas9, can also be used according to some embodiments together with other enzymes (possibly as a fusion protein) for base-editing. Base editing is a genome editing approach that uses components from CRISPR systems together with other enzymes to directly install point mutations into cellular DNA or RNA without making double-stranded DNA breaks. DNA base editors comprise a catalytically disabled nuclease fused to a nucleobase deaminase enzyme and, in some cases, a DNA glycosylase inhibitor. RNA base editors achieve analogous changes using components that target RNA. Base editors directly convert one base or base pair into another, enabling the efficient installation of point mutations in non-dividing cells without generating excess undesired editing by-products (Rees and Liu (2018), “Base Editing: Precision Chemistry on the Genome and Transcriptome of Living Cells”, Nature Reviews Genetics, 19(12): 770-788). According to some embodiments, the modified Cas9 is an nCas fused to a base editor enzyme such as an adenosine or cytidine deaminase. Particular base editors contemplated include APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, ABE7.10*, xABE, ABESa, VQR-ABE, VRER-ABE, SaKKH-ABE (Rees and Liu (2018), “Base Editing: Precision Chemistry on the Genome and Transcriptome of Living Cells”, Nature Reviews Genetics, 19(12): 770-788, and references therein).

As used herein, the “guide RNA” (or “gRNA”) is not limited to a particular sequence, provided that the sequence is either specific to the at least one polyphenol oxidase gene, or targeted to a genomic sequence that encodes a silencing RNA, whose sequence is changed such that the encoded silencing RNA silences a polyphenol oxidase gene as defined herein. In some embodiments of the invention, the one or more guide RNAs comprises a variable region having a sequence selected from the group consisting of: SEQ ID NOs: 32-39 and 57-97; and any combination of the foregoing. In some embodiments of the invention, the one or more guide RNAs comprises a variable region having a sequence selected from the group consisting of: SEQ ID NOs: 180-196, 199-202 and 207-225; and any combination of the foregoing. In some embodiments of the invention, the one or more guide RNAs comprises a variable region having a sequence selected from the group consisting of: SEQ ID NO: 32; SEQ ID NO: 33; SEQ ID NO: 34; SEQ ID NO: 35; SEQ ID NO: 36; SEQ ID NO: 37; SEQ ID NO: 57; SEQ ID NO: 58; SEQ ID NO: 38;

SEQ ID NO: 39; SEQ ID NO: 62; SEQ ID NO: 76; SEQ ID NO: 77; and any combination of the foregoing.

In some embodiments of the invention, said one or more guide RNAs are a pair of guide RNAs comprising variable regions selected from the group consisting of: SEQ ID NOs: 32 and 33; SEQ ID NOs: 34 and 35; SEQ ID NOs: 32 and 34; SEQ ID NOs: 32 and 35; SEQ ID NOs: 33 and 34; SEQ ID NOs: 33 and 35; SEQ ID NOs: 57 and 58; SEQ ID NOs: 38 and 39; SEQ ID NOs: 62 and 33; and SEQ ID NOs: 76 and 77. According to some embodiments, the above gRNAs comprise, a variable region sequence as set forth above, followed by a constant scaffold sequence of SEQ ID NO: 98.

Preferably, the invention provides a method of reducing the level or activity of at least one endogenous PPO1 polyphenol oxidase encoded by a PPO1 polyphenol oxidase gene in a banana plant or banana plant cell, wherein the PPO1 polyphenol oxidase gene: (A) comprises a coding sequence of SEQ ID NO: 5 (PPO1) or a polynucleotide with at least 75% sequence identity to SEQ ID NO: 5; (B) encodes a polyphenol oxidase of SEQ ID NO: 40 (PPO1) or a polypeptide with at least 75% sequence identity to SEQ ID NO: 40; or (C) comprises a polynucleotide sequence of SEQ ID NO: 151 (PPO1) or a polynucleotide with at least 75% sequence identity to SEQ ID NO: 151;
wherein the method comprises providing to said banana plant cell a CRISPR-associated endonuclease or a modified CRISPR-associated endonuclease, and one or more guide RNAs specific to said at least one PPO1 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO1 polyphenol oxidase gene;
optionally wherein said one or more guide RNAs comprises a variable region having a sequence of SEQ ID NO: 32, SEQ ID NO: 33, or SEQ ID NO: 62.

Preferably, the invention provides a method of reducing the level or activity of at least one endogenous PPO2 polyphenol oxidase encoded by a PPO2 polyphenol oxidase gene in a banana plant or banana plant cell, wherein the PPO2 polyphenol oxidase gene: (A) comprises a coding sequence of SEQ ID NO: 6 (PPO2) or a polynucleotide with at least 75% sequence identity to SEQ ID NO: 6; (B) encodes a polyphenol oxidase of SEQ ID NO: 41 (PPO2) or a polypeptide with at least 75% sequence identity to SEQ ID NO: 41; or (C) comprises a polynucleotide sequence of SEQ ID NO: 152 (PPO2) or a polynucleotide with at least 75% sequence identity to SEQ ID NO: 152;
wherein the method comprises providing to said banana plant cell a CRISPR-associated endonuclease or a modified CRISPR-associated endonuclease, and one or more guide RNAs specific to said at least one PPO2 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO2 polyphenol oxidase gene;
optionally wherein said one or more guide RNAs comprises a variable region having a sequence of SEQ ID NO: 34 or SEQ ID NO: 35.

Preferably, the invention provides a method of reducing the level or activity of at least one endogenous PPO5 polyphenol oxidase encoded by a PPO5 polyphenol oxidase gene in a banana plant or banana plant cell, wherein the PPO5 polyphenol oxidase gene: (A) comprises a coding sequence of SEQ ID NO: 9 (PPO5) or a polynucleotide with at least 75% sequence identity to SEQ ID NO: 9; (B) encodes a polyphenol oxidase of SEQ ID NO: 44 (PPO5) or a polypeptide with at least 75% sequence identity to SEQ ID NO: 44; or (C) comprises a polynucleotide sequence of SEQ ID NO: 155 (PPO5) or a polynucleotide with at least 75% sequence identity to SEQ ID NO: 155;
wherein the method comprises providing to said banana plant cell a CRISPR-associated endonuclease or a modified CRISPR-associated endonuclease, and one or more guide RNAs specific to said at least one PPO5 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO5 polyphenol oxidase gene;
optionally wherein said one or more guide RNAs comprises a variable region having a sequence of SEQ ID NO: 78 or SEQ ID NO: 79.

Preferably, the invention provides a method of reducing the level or activity of at least one endogenous PPO8 polyphenol oxidase encoded by a PPO8 polyphenol oxidase gene in a banana plant or banana plant cell, wherein the PPO8 polyphenol oxidase gene: (A) comprises a coding sequence of SEQ ID NO: 12 (PPO8) or a polynucleotide with at least 75% sequence identity to SEQ ID NO: 12; (B) encodes a polyphenol oxidase of SEQ ID NO: 47 (PPO8) or a polypeptide with at least 75% sequence identity to SEQ ID NO: 47; or (C) comprises a polynucleotide sequence of SEQ ID NO: 158 (PPO8) or a polynucleotide with at least 75% sequence identity to SEQ ID NO: 158;
wherein the method comprises providing to said banana plant cell a CRISPR-associated endonuclease or a modified CRISPR-associated endonuclease, and one or more guide RNAs specific to said at least one PPO8 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO8 polyphenol oxidase gene;
optionally wherein said one or more guide RNAs comprises a variable region having a sequence of SEQ ID NO: 57 or SEQ ID NO: 58.

Preferably, the invention provides a method of reducing the level or activity of at least one endogenous PPO9 polyphenol oxidase encoded by a PPO9 polyphenol oxidase gene in a banana plant or banana plant cell, wherein the PPO9 polyphenol oxidase gene: (A) comprises a coding sequence of SEQ ID NO: 13 (PPO9) or a polynucleotide with at least 75% sequence identity to SEQ ID NO: 13; (B) encodes a polyphenol oxidase of SEQ ID NO: 48 (PPO9) or a polypeptide with at least 75% sequence identity to SEQ ID NO: 48; or (C) comprises a polynucleotide sequence of SEQ ID NO: 159 (PPO9) or a polynucleotide with at least 75% sequence identity to SEQ ID NO: 159;
wherein the method comprises providing to said banana plant cell a CRISPR-associated endonuclease or a modified CRISPR-associated endonuclease, and one or more guide RNAs specific to said at least one PPO9 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO9 polyphenol oxidase gene;
optionally wherein said one or more guide RNAs comprises a variable region having a sequence of SEQ ID NO: 38 or SEQ ID NO: 39.

Preferably, the invention provides a method of reducing the level or activity of at least one endogenous PPO4 polyphenol oxidase encoded by a PPO4 polyphenol oxidase gene in a banana plant or banana plant cell, wherein the PPO4 polyphenol oxidase gene: (A) comprises a coding sequence of SEQ ID NO: 8 (PPO4) or a polynucleotide with at least 75% sequence identity to SEQ ID NO: 8; (B) encodes a polyphenol oxidase of SEQ ID NO: 43 (PPO4) or a polypeptide with at least 75% sequence identity to SEQ ID NO: 43; or (C) comprises a polynucleotide sequence of SEQ ID NO: 154 (PPO4) or a polynucleotide with at least 75% sequence identity to SEQ ID NO: 154;
wherein the method comprises providing to said banana plant cell a CRISPR-associated endonuclease or a modified CRISPR-associated endonuclease, and one or more guide RNAs specific to said at least one PPO4 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO4 polyphenol oxidase gene;
optionally wherein said one or more guide RNAs comprises a variable region having a sequence of SEQ ID NO: 76 or SEQ ID NO: 77. In order to use the CRISPR/Cas system, both gRNA and Cas should be in a target cell or delivered as a ribonucleoprotein complex. According to some embodiments, the Cas/modified Cas and at least one gRNA are provided to a banana cell by introducing one or more vectors which express the Cas/modified Cas and/or the at least one gRNA. The insertion vector can contain both cassettes on a single plasmid, or the cassettes are expressed from two separate plasmids. CRISPR plasmids are commercially available (such as the px330 plasmid from Addgene). The use of clustered regularly interspaced short palindromic repeats (CRISPR)-associated (Cas)-guide RNA technology and a Cas endonuclease for modifying plant genomes are also at least disclosed by Svitashev et al. (2015), Plant Physiology, 169 (2): 931-945; Kumar and Jain, 2015, Journal of Experimental Botany, 66: 47-57; and in U.S. Patent Application Publication No. 20150082478, which is specifically incorporated herein by reference in its entirety.

In some embodiments, the method of the invention further comprises identifying at least one banana plant cell that comprises a modification of the at least one endogenous polyphenol oxidase gene. In some embodiments, the method of the invention further comprises identifying at least one banana plant cell that comprises a modification of the at least one endogenous polyphenol oxidase gene, wherein the modification is selected from the group consisting of: at least one nucleotide insertion; at least one nucleotide deletion; at least one nucleotide substitution; and any combination of the foregoing. In some of these embodiments, the at least one endogenous polyphenol oxidase gene is selected from the group consisting of: PPO1, PPO2, PPO3, PPO4, PPO5, PPO6, PPO7, PPO8, and PPO9. In some embodiments, the at least one endogenous polyphenol oxidase gene is a PPO1 gene. In some embodiments, the at least one endogenous polyphenol oxidase gene is a PPO2 gene. In some embodiments, the at least one endogenous polyphenol oxidase gene is a PPO3 gene. In some embodiments, the at least one endogenous polyphenol oxidase gene is a PPO4 gene. In some embodiments, the at least one endogenous polyphenol oxidase gene is a PPO5 gene. In some embodiments, the at least one endogenous polyphenol oxidase gene is a PPO6 gene. In some embodiments, the at least one endogenous polyphenol oxidase gene is a PPO7 gene. In some embodiments, the at least one endogenous polyphenol oxidase gene is a PPO8 gene. In some embodiments, the at least one endogenous polyphenol oxidase gene is a PPO9 gene. In some embodiments, the endogenous polyphenol oxidase genes are two or more of PPO1, PPO2, PPO3, PPO4, PPO5, PPO6, PPO7, PPO8, and PPO9 genes. In some embodiments, the endogenous polyphenol oxidase genes are PPO1 and PPO2 genes.

As used herein, “identifying” can include any technique known in the art capable of detecting the modification or editing event selected from the group consisting of: at least one nucleotide insertion; at least one nucleotide deletion; at least one nucleotide substitution; and any combination of the foregoing, such as, but not limited to, DNA sequencing (e.g. next generation sequencing), electrophoresis, an enzyme-based mismatch-detection assay, and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based 17 endonuclease, Heteroduplex and Sanger sequencing. Another method of validating the presence of a DNA editing event (such as insertion-deletion events (or “indels”)) comprises a mismatch cleavage assay that makes use of a structure selective enzyme (e.g. endonuclease) that recognizes and cleaves mismatched DNA. The mismatch cleavage assay is a simple and cost-effective method for the detection of indels and is therefore the typical procedure to detect mutations induced by genome editing. The assay uses enzymes that cleave heteroduplex DNA at mismatches and extrahelical loops formed by multiple nucleotides, yielding two or more smaller fragments. A PCR product of about 300 to 1000 bp is generated with the predicted nuclease cleavage site off-centre so that the resulting fragments are dissimilar in size and can easily be resolved by conventional gel electrophoresis or high-performance liquid chromatography (HPLC). End-labelled digestion products can also be analysed by automated gel or capillary electrophoresis. The frequency of indels at the locus can be estimated by measuring the integrated intensities of the PCR amplicon and cleaved DNA bands. The digestion step takes 15 to 60 minutes, and when the DNA preparation and PCR steps are added, the entire assays can be completed in less than 3 hours. Two alternative enzymes are typically used in this assay. 17 endonuclease 1 (T7E1) is a resolvase that recognises and cleaves imperfectly matched DNA at the first, second or third phosphodiester bond upstream of the mismatch. The sensitivity of a T7E1-based assay is 0.5 to 5%. In contrast, Surveyor™ nuclease (Transgenomic Inc., Omaha, NE, USA) is a member of the CEL family of mismatch-specific nucleases derived from celery. It recognizes and cleaves mismatches due to the presence of single nucleotide polymorphisms (SNPs) or small indels, cleaving both DNA strands downstream of the mismatch. It can detect indels of up to 12 nucleotides and is sensitive to mutations present at frequencies as low as about 3%, i.e. 1 in 32 copies. Yet another method of validating the presence of an editing event comprises the high-resolution melting analysis. High-resolution melting analysis (HRMA) involves the amplification of a DNA sequence spanning the genomic target (90 to 200 bp) by real-time PCR with the incorporation of a fluorescent dye, followed by melt curve analysis of the amplicons. HRMA is based on the loss of fluorescence when intercalating dyes are released from double-stranded DNA during thermal denaturation. It records the temperature-dependent denaturation profile of amplicons and detects whether the melting process involves one or more molecular species. Yet another method is the heteroduplex mobility assay. Mutations can also be detected by analysing re-hybridized PCR fragments directly by native polyacrylamide gel electrophoresis (PAGE). This method takes advantage of the differential migration of heteroduplex and homoduplex DNA in polyacrylamide gels. The angle between matched and mismatched DNA strands caused by an indel means that heteroduplex DNA migrates at a significantly slower rate than homoduplex DNA under native conditions, and they can easily be distinguished based on their mobility. Fragments of 140 to 170 bp can be separated in a 15% polyacrylamide gel. The sensitivity of such assays can approach 0.5% under optimal conditions, which is similar to T7E1. After reannealing the PCR products, the electrophoresis component of the assay takes about 2 hours. Other methods of validating the presence of editing events are described in length in Zischewski (2017), Biotechnology Advances 1(1): 95-104.

In some embodiments of the invention, the one or more guide RNAs (gRNAs) are provided to the banana plant cell within one or more recombinant DNA constructs encoding said one or more guide RNAs operably linked to one or more promoters. DNA constructs useful in the embodiments of the invention may be constructed using recombinant DNA technology well known to a person skilled in the art. Such DNA constructs may be commercially available, suitable for transforming into plants and suitable for expression of the gene of interest in the transformed cells.

As used herein, the “promoter” is plant-expressible, i.e. capable of inducing, conferring, activating or enhancing expression in a plant cell, tissue or organ. Examples of promoters useful in the methods of the invention include, but are not limited to, Actin, CANV 35S, CaMV19S, GOS2. Promoters active in various tissues or developmental stages can also be used. In any of the embodiments herein, PPO polynucleotide sequences may be optimised for plant expression. Examples of such sequence modifications include, but are not limited to, an altered G/C content to more closely approach that typically found in banana, and the removal of codons atypically found in the plant species commonly (referred to as codon optimisation). Banana plant cells may be transformed stably or transiently with the DNA constructs of the embodiments of the invention. In stable transformation, the PPO polynucleotide is integrated into the plant genome and as such it represents a stable and inherited trait. In transient transformation, the PPO polynucleotide is expressed by the cell transformed, but not integrated into the genome (and as such, it represents a transient trait). In some embodiments, the promoter in the DNA construct comprises a Pol3 promoter. Examples of Pol3 promoters include, but are not limited to, AtU6-29, AtU626, AtU3B, AtU3d, and TaU6. In some embodiments, the promoter in the DNA construct comprises a Pol2 promoter. Examples of Pol2 promoters include, but are not limited to, CaMV 35S, CaMV 195, ubiquitin, CVMV. In some embodiments, the promoter in the DNA construct comprises a 35S promoter. In some embodiments, the promoter in the DNA construct comprises a U6 promoter. In some embodiments, the promoter in the DNA construct comprises a Pol3 promoter (such as U6) operatively linked to the nucleic acid agent encoding at least one gRNA and/or a Pol2 promoter (such as CamV35S) operatively linked to the nucleic acid sequence encoding the CRISPR-associated endonuclease and/or a selectable marker gene. The DNA construct may be useful for transient expression by Agrobacterium-mediated transformation (Helens et al. (2005), Plant Methods 1: 13). In some embodiments, the nucleic acid sequences comprised in the DNA construct are devoid of sequences which are homologous to the genome of the banana plant cell (other than any guide sequences), so as to avoid integration into the banana genome. In some embodiments, the DNA construct is a non-integrating construct, such as where the nucleic acid sequence encoding the selectable marker is also non-integrating. As used herein, “non-integrating” refers to a DNA construct or sequence that is not affirmatively designed to facilitate integration of the construct or sequence into the genome of the plant of interest. For example, a functional T-DNA vector system for Agrobacterium-mediated genetic transformation is not a non-integrating vector system, as the system is affirmatively designed to integrate into the plant genome. Similarly, a selectable marker gene sequence that has flanking sequences homologous to the genome of the plant of interest to facilitate homologous recombination of the selectable marker gene sequence into the banana genome would not be a non-integrating selectable marker sequence.

Various cloning kits can be used in the context of the invention. As used herein, the “DNA construct” may be a binary vector. Examples for binary vectors are pBIN19, pBHOI, pBinAR, pGPTV, pCAMBIA, pBIB-HYG, pBecks, pGreen or pPZP (Hajukiewicz, P. et al., Plant Molecular Biology, 25, 989 (1994), and Hellens et al. Trends in Plant Science 5, 446 (2000)). Examples of other vectors that can be used in the context of the present invention in other methods of DNA delivery (e.g. transfection, electroporation, particle bombardment, and viral inoculation) are: pGE-sgRNA (Zhang et al. Nature Communications 2016 7: 12697), pJIT163-Ubi-Cas9 (Wang et al. Nature Biotechnology 2004, 32, 947-951), pICH47742:: 2x35S-5′UTR-hCas9(STOP)-NOST (Belhan et al. Plant Methods 2013, 11; 9(1): 39).

In other embodiments, said CRISPR-associated endonuclease or modified CRISPR-associated endonuclease, and/or the one or more guide RNAs, are provided to the banana plant cell in RNA form. In yet other embodiments, the CRISPR-associated endonuclease or modified CRISPR-associated endonuclease is provided to the banana plant cell in protein form and the one or more guide RNAs are provided to said banana plant cell in RNA form. In some embodiments, the CRISPR-associated endonuclease or modified CRISPR-associated endonuclease and the one or more guide RNAs are provided to the banana plant cell as a ribonucleoprotein complex.

There are a number of methods of introducing DNA, RNA, peptides and/or proteins or combinations of nucleic acids and peptides into plant cells. These include, for example, protoplast transformation (U.S. Pat. No. 5,508,184); desiccation/inhibition-mediated DNA uptake (Potrykus et al. (1985) Mol. Gen. Genet. 199: 183-8); electroporation (U.S. Pat. No. 5,384,253); agitation with silicon carbide fibres (U.S. Pat. Nos. 5,302,523 and 5,464,765); Agrobacterium-mediated transformation (U.S. Pat. Nos. 5,563,055, 5,591,616, 5,693,512, 5,824,877, 5,981,840, and 6,384,301); acceleration of DNA-coated particles (U.S. Pat. Nos. 5,015,580, 5,550,318, 5,538,880, 6,160,208, 6,399,861, and 6,403,865) and nanoparticles, nanocarriers and cell penetrating peptides (WO201126644A2; WO2009046384A1; WO2008148223A1). Other methods of transfection include the use of transfection reagents (e.g. Lipofectin, ThermoFisher), dendrimers (Kukowska-Latallo, J. F. et al. (1996), Proc. Natl. Acad. Sci. USA 93, 4897-902), cell penetrating peptides (Mae et al. (2005), “Internalisation of cell-penetrating peptides into tobacco protoplasts”, Biochimica et Biophysica Acta 1669(2): 101-7) or polyamines (Zhang and Vinogradov (2010), “Short biodegradable polyamines for gene delivery and transfection of brain capillary endothelial cells”, J Control Release, 143(3):359-366).

In some embodiments of the invention, the endonuclease, the one or more guide RNAs and/or the one or more recombinant DNA constructs are provided to the banana plant cell using particle bombardment or biolistics. In other embodiments, the endonuclease, the one or more guide RNAs and/or the one or more recombinant DNA constructs are provided to the banana plant cell using Agrobacterium transformation.

In yet other embodiments, the endonuclease, the one or more guide RNAs and/or the one or more recombinant DNA constructs are provided to the banana plant cell using protoplast transfection. In yet other embodiments, the endonuclease, the one or more guide RNAs and/or the one or more recombinant DNA constructs are provided to the banana plant cell using electroporation. In yet other embodiments, the endonuclease, the one or more guide RNAs and/or the one or more recombinant DNA constructs are provided to the banana plant cell using nanoparticle-mediated transfection. In some embodiments, the endonuclease is provided to the banana plant cell as a polynucleotide encoding an endonuclease polypeptide. In some of these embodiments, the endonuclease is Cas9 endonuclease and the polynucleotide is a Cas9 polynucleotide encoding a Cas9 polypeptide. In some embodiments, the one or more guide RNAs and the CRISPR-associated endonuclease or modified CRISPR-associated endonuclease are provided to the banana plant cell via Agrobacterium transformation of one or more plasmids encoding the CRISPR-associated endonuclease or modified CRISPR-associated endonuclease, at least one selectable marker gene, and the one or more guide RNAs.

In any of the embodiments of the invention, the one or more guide RNAS comprises a variable region having a sequence selected from the group consisting of: SEQ ID NOs: 32-39 and 57-97; and any combination of the foregoing. In some embodiments of the invention, the one or more guide RNAs comprises a variable region having a sequence selected from the group consisting of: SEQ ID NOs: 180-196, 199-202 and 207-225; and any combination of the foregoing. In any of the embodiments of the invention, the one or more guide RNAs comprises a variable region having a sequence selected from the group consisting of: SEQ ID NO: 32; SEQ ID NO: 33; SEQ ID NO: 34; SEQ ID NO: 35; SEQ ID NO: 36; SEQ ID NO: 37; SEQ ID NO: 57; SEQ ID NO: 58; SEQ ID NO: 38; SEQ ID NO: 39; SEQ ID NO: 62; SEQ ID NO: 76; SEQ ID NO: 77; and any combination of the foregoing. In some embodiments of the invention, said one or more guide RNAs are a pair of guide RNAs comprising variable regions selected from the group consisting of: SEQ ID NOs: 32 and 33; SEQ ID NOs: 34 and 35; SEQ ID NOs: 32 and 34; SEQ ID NOs: 32 and 35; SEQ ID NOs: 33 and 34; SEQ ID NOs: 33 and 35; SEQ ID NOs: 57 and 58; SEQ ID NOs: 38 and 39; SEQ ID NOs: 62 and 33; and SEQ ID NOs: 76 and 77.

In some embodiments of the invention, the banana plant cell is a protoplast, an embryogenic cell, and/or contained in an embryogenic cell suspension.

The invention further provides a banana plant cell obtainable by any one of the foregoing methods of the invention.

In some embodiments of the invention, the method further comprises regenerating a banana plant from said banana plant cell.

As used herein, “regenerating” may comprise growing banana plant cells (which include protoplasts) into whole banana plants by first growing the banana plant cells into groups that develop into a callus, followed by the regeneration of shoots (caulogenesis) from the callus using plant tissue culture methods. The growth of banana protoplasts into callus and subsequent regeneration of shoots requires the proper balance of plant growth regulators in the tissue culture medium that must be customised. Protoplasts may also be used for plant breeding, using a technique called protoplast fusion. Protoplasts from different species are induced to fuse by using an electric field or a solution of polyethylene glycol. This technique may be used to generate somatic hybrids in tissue culture. Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment (see Maheshwari et al. (1986), “Differentiation of Protoplasts and of Transformed Plant Cells”: 3-36. Springer-Verlag, Berlin). The regenerated banana plants can be subjected to selection. The banana plant or cells thereof may be devoid of a transgene, i.e. “non-transgenic”. For example, the banana plants may be devoid of any of the DNA constructs encoding any of the CRISPR/Cas system as used in some of the embodiments of the invention. According to some embodiments, when performing genetic manipulations such as genetic editing on banana cells to be grown and regenerated into an adult plant, it is preferable not to edit genes which may be expressed in these cells and negatively affect embryo formation and/or regeneration. Without wishing to be bound by theory or mechanism, the discovery of PPO genes which are expressed in fruit flesh and/or peel but are not expressed in embryonic cells, such as PPO1, PPO2, PPO8, PPO9, or PPO4, enables editing embryonic cells without affecting plant regeneration.

In some of embodiments, the method of the invention further comprises harvesting fruit from said banana plant. Each adult banana plant produces a single bunch, which is formed by many banana fruits or “fingers” and clustered in several “hands”. In this regard, “harvesting” has the conventional meaning. For example, banana bunches may be cut by hand (usually involving 2 or 3 people) using a sharp curved knife or a machete. The harvest usually occurs when the banana fruits are still green and firm, 7 to 14 days prior to ripening.

The invention further provides a banana plant or plant part obtainable by the foregoing method.

Yet further provided by the invention is fruit harvested from a banana plant obtainable by the foregoing method of the invention, wherein said fruit flesh and/or fruit peel is characterised by a phenotype of delayed and/or reduced browning as compared to fruit flesh and/or fruit peel of a banana plant in which the level or activity of said at least one endogenous polyphenol oxidase (selected from the group consisting of: PPO1, PPO2, PPO3, PPO4, PPO5, PPO6, PPO7, PPO8, and PPO9) is not reduced.

According to some embodiments, provided herein is fruit harvested from a banana plant obtainable by the foregoing method of the invention, wherein said fruit flesh and/or fruit peel is characterised by a phenotype of delayed and/or reduced browning as compared to fruit flesh and/or fruit peel of a banana plant in which the level or activity of said at least one endogenous polyphenol oxidase (selected from the group consisting of: PPO1, PPO2, PPO8, PPO9, and PPO4) is not reduced.

The invention further provides a method of producing a banana plant characterised by a phenotype of delayed and/or reduced browning of fruit flesh and/or peel as compared to a wild-type banana plant, the method comprising: providing to a banana plant cell a CRISPR-associated endonuclease or a modified CRISPR-associated endonuclease, and one or more guide RNAs, wherein the CRISPR-associated endonuclease or modified CRISPR-associated endonuclease and the one or more guide RNAs form a complex that enables the CRISPR-associated endonuclease or modified CRISPR-associated endonuclease to introduce a single-strand break or a double-strand break in at least one endogenous polyphenol oxidase gene selected from the group consisting of: PPO1, PPO2, PPO3, PPO4, PPO5, PPO6, PPO7, PPO8, and PPO9; identifying at least one banana plant cell that comprises a modification of the at least one endogenous polyphenol oxidase gene selected from the group consisting of: PPO1, PPO2, PPO3, PPO4, PPO5, PPO6, PPO7, PPO8, and PPO9, wherein the modification is selected from the group consisting of: at least one nucleotide insertion; at least one nucleotide deletion; at least one nucleotide substitution; or any combination of the foregoing; and regenerating a banana plant from the banana plant cell, wherein the banana plant is characterised by a phenotype of delayed and/or reduced browning of fruit flesh and/or peel as compared to a wild-type banana plant. In some embodiments, the method further comprises harvesting fruit from said banana plant, wherein the fruit is characterised by a phenotype of delayed and/or reduced browning as compared to fruit of a banana plant in which the level or activity of the at least one endogenous polyphenol oxidase is not reduced or lost.

The invention further provides a banana plant or plant part comprising in its genome at least one modified endogenous polyphenol oxidase gene, wherein the modification results in a reduction in, or loss of function of, the at least one endogenous polyphenol oxidase encoded by the modified endogenous polyphenol oxidase gene, wherein the modification is located in at least one endogenous polyphenol oxidase gene. In some embodiments of the foregoing method, banana plant, or banana plant part, the at least one endogenous polyphenol oxidase gene is a PPO1 gene. In some embodiments, the at least one endogenous polyphenol oxidase gene is a PPO2 gene. In some embodiments, the at least one endogenous polyphenol oxidase gene is a PPO3 gene. In some embodiments, the at least one endogenous polyphenol oxidase gene is a PPO4 gene. In some embodiments, the at least one endogenous polyphenol oxidase gene is a PPO5 gene. In some embodiments, the at least one endogenous polyphenol oxidase gene is a PPO6 gene. In some embodiments, the at least one endogenous polyphenol oxidase gene is a PPO7 gene. In some embodiments, the at least one endogenous polyphenol oxidase gene is a PPO8 gene. In some embodiments, the at least one endogenous polyphenol oxidase gene is a PPO9 gene. In some embodiments, the endogenous polyphenol oxidase genes are two or more of PPO1, PPO2, PPO3, PPO4, PPO5, PPO6, PPO7, PPO8, and PPO9 genes. In some embodiments, the endogenous polyphenol oxidase genes are PPO1 and PPO2 genes. In particular embodiments of the foregoing method, banana plant, or banana plant part, the PPO1 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5; the PPO2 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6; the PPO3 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7; the PPO4 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8; the PPO5 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9; the PPO6 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10; the PPO7 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11; the PPO8 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12; or the PPO9 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13. In particular embodiments of the foregoing method, banana plant, or banana plant part, the PPO1, PPO2, PPO8, PPO9, or PPO4 polyphenol oxidase gene polynucleotide comprises a coding sequence that is selected from the group consisting of SEQ ID NO: 5 (PPO1) or a polynucleotide with at least 75% sequence identity to SEQ ID NO: 5; SEQ ID NO: 6 (PPO2) or a polynucleotide with at least 75% sequence identity to SEQ ID NO: 6; SEQ ID NO: 12 (PPO8) or a polynucleotide with at least 75% sequence identity to SEQ ID NO: 12; SEQ ID NO: 13 (PPO9) or a polynucleotide with at least 75% sequence identity to SEQ ID NO: 13; and SEQ ID NO: 8 (PPO4) or a polynucleotide with at least 75% sequence identity to SEQ ID NO: 8.

In particular embodiments of the foregoing method, banana plant, or banana plant part, the PPO1 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40; the PPO2 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41; the PPO3 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42; the PPO4 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43; the PPO5 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44; the PPO6 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45; the PPO7 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46; the PPO8 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47; or the PPO9 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In particular embodiments of the foregoing method, banana plant, or banana plant part, the PPO1, PPO2, PPO8, or PPO9 polyphenol oxidase gene encodes a polyphenol oxidase selected from the group consisting of: SEQ ID NO: 40 (PPO1) or a polypeptide with at least 75% sequence identity to SEQ ID NO: 40; SEQ ID NO: 41 (PPO2) or a polypeptide with at least 75% sequence identity to SEQ ID NO: 41; SEQ ID NO: 47 (PPO8) or a polypeptide with at least 75% sequence identity to SEQ ID NO: 47; SEQ ID NO: 48 (PPO9) or a polypeptide with at least 75% sequence identity to SEQ ID NO: 48; and SEQ ID NO: 43 (PPO4) or a polypeptide with at least 75% sequence identity to SEQ ID NO: 43.

In particular embodiments of the foregoing method, banana plant, or banana plant part, the PPO1 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151; the PPO2 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152; the PPO3 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153; the PPO4 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154; the PPO5 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155; the PPO6 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156; the PPO7 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157; the PPO8 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158; or the PPO9 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159. In particular embodiments of the foregoing method, banana plant, or banana plant part, the PPO1, PPO2, PPO8, PPO9, or PPO4 polyphenol oxidase gene comprises a polynucleotide sequence selected from the group consisting of: SEQ ID NO: 151 (PPO1) or a polynucleotide with at least 75% sequence identity to SEQ ID NO: 151; SEQ ID NO: 152 (PPO2) or a polynucleotide with at least 75% sequence identity to SEQ ID NO: 152; SEQ ID NO: 158 (PPO8) or a polynucleotide with at least 75% sequence identity to SEQ ID NO: 158; SEQ ID NO: 159 (PPO9) or a polynucleotide with at least 75% sequence identity to SEQ ID NO: 159; and SEQ ID NO: 154 (PPO4) or a polynucleotide with at least 75% sequence identity to SEQ ID NO: 154.

In some embodiments, the banana plant or plant part of the present invention is non-transgenic. For example, the banana plants or plant parts of the present invention may be devoid of any of the DNA constructs encoding any of the CRISPR/Cas system as used in some of the embodiments of the invention.

Yet further provided by the invention is banana fruit harvested from the banana plant of any of the embodiments of the invention, wherein the fruit is characterised by a phenotype of delayed and/or reduced browning as compared to fruit of a banana plant in which the level or activity of said at least one endogenous polyphenol oxidase selected from the group consisting of: PPO1, PPO2, PPO3, PPO4, PPO5, PPO6, PPO7, PPO8, and PPO9 is not reduced.

The invention further provides a method of obtaining a banana fruit food product, the method comprising processing the banana fruit of the invention. In some embodiments, the banana fruit food product is a thickening agent, colouring, or flavour. Also contemplated are livestock feed, natural fibres, and sources of natural bioactive compounds and bio-fertilisers.

The invention further provides a DNA sequence comprising a banana polyphenol oxidase polynucleotide.

Yet further provided by the invention is a DNA construct or vector comprising a banana polyphenol oxidase polynucleotide.

The invention further provides a plant cell transformed with a vector comprising a banana polyphenol oxidase polynucleotide. In some of these embodiments, the plant cell is a banana plant cell.

Yet further provided by the invention is a polyphenol oxidase protein. In some embodiments, the polyphenol oxidase protein is selected from the group consisting of: PPO1, PPO2, PPO3, PPO4, PPO5, PPO6, PPO7, PPO8, and PPO9. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47. In some embodiments, the polyphenol oxidase protein comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158. In some embodiments, the polyphenol oxidase protein is encoded by a polynucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159.

The invention further provides a method of expressing a polyphenol oxidase in a plant cell, wherein the method comprises introducing into said plant cell a banana polyphenol oxidase polynucleotide; operably linked to a promoter active in a plant cell.

In some of the embodiments of the DNA sequence, DNA construct, vector, plant cell transformed with a vector, banana plant cell transformed with a vector, and method of expressing a polyphenol oxidase in a plant cell, the polyphenol oxidase polynucleotide is selected from the group consisting of: PPO1, PPO2, PPO3, PPO4, PPO5, PPO6, PPO7, PPO8, and PPO9. In some embodiments of the DNA sequence, DNA construct, vector, plant cell transformed with a vector, banana plant cell transformed with a vector, and method of expressing a polyphenol oxidase in a plant cell, the at least one endogenous polyphenol oxidase polynucleotide is PPO1 polynucleotide. In some embodiments, the polyphenol oxidase polynucleotide is a PPO2 polynucleotide. In some embodiments, the polyphenol oxidase polynucleotide is a PPO3 polynucleotide. In some embodiments, the polyphenol oxidase polynucleotide is a PPO4 polynucleotide. In some embodiments, the polyphenol oxidase polynucleotide is a PPO5 polynucleotide. In some embodiments, the polyphenol oxidase polynucleotide is a PPO6 polynucleotide. In some embodiments, the polyphenol oxidase polynucleotide is a PPO7 polynucleotide. In some embodiments, the polyphenol oxidase polynucleotide is a PPO8 polynucleotide. In some embodiments, the polyphenol oxidase polynucleotide is a PPO9 polynucleotide. In some embodiments, the polyphenol oxidase polynucleotide comprises two or more of PPO1, PPO2, PPO3, PPO4, PPO5, PPO6, PPO7, PPO8, and PPO9 polynucleotides. In some embodiments of the DNA sequence, DNA construct, vector, plant cell transformed with a vector, banana plant cell transformed with a vector, and method of expressing a polyphenol oxidase in a plant cell, the polyphenol oxidase polynucleotides are PPO1 and PPO2 polynucleotides. In particular embodiments of the DNA sequence, DNA construct, vector, plant cell transformed with a vector, banana plant cell transformed with a vector, and method of expressing a polyphenol oxidase in a plant cell, the PPO1 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5; the PPO2 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6; the PPO3 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7; the PPO4 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8; the PPO5 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9; the PPO6 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10; the PPO7 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11; the PPO8 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12; or the PPO9 polynucleotide comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13. In particular embodiments of the DNA sequence, DNA construct, vector, plant cell transformed with a vector, banana plant cell transformed with a vector, and method of expressing a polyphenol oxidase in a plant cell, the PPO1, PPO2, PPO8, PPO9, or PPO4 polyphenol oxidase polynucleotide comprises a coding sequence that is selected from the group consisting of SEQ ID NO: 5 (PPO1) or a polynucleotide with at least 75% sequence identity to SEQ ID NO: 5; SEQ ID NO: 6 (PPO2) or a polynucleotide with at least 75% sequence identity to SEQ ID NO: 6; SEQ ID NO: 12 (PPO8) or a polynucleotide with at least 75% sequence identity to SEQ ID NO: 12; SEQ ID NO: 13 (PPO9) or a polynucleotide with at least 75% sequence identity to SEQ ID NO: 13; and SEQ ID NO: 8 (PPO4) or a polynucleotide with at least 75% sequence identity to SEQ ID NO: 8.

In particular embodiments of the DNA sequence, DNA construct, vector, plant cell transformed with a vector, banana plant cell transformed with a vector, and method of expressing a polyphenol oxidase in a plant cell, the PPO1 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40; the PPO2 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41; the PPO3 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42; the PPO4 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43; the PPO5 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44; the PPO6 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45; the PPO7 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46; the PPO8 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47; or the PPO9 polynucleotide encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In particular embodiments of the DNA sequence, DNA construct, vector, plant cell transformed with a vector, banana plant cell transformed with a vector, and method of expressing a polyphenol oxidase in a plant cell, the PPO1, PPO2, PPO8, PPO9, or PPO4 polyphenol oxidase polynucleotide encodes a polyphenol oxidase selected from the group consisting of: SEQ ID NO: 40 (PPO1) or a polypeptide with at least 75% sequence identity to SEQ ID NO: 40; SEQ ID NO: 41 (PPO2) or a polypeptide with at least 75% sequence identity to SEQ ID NO: 41; SEQ ID NO: 47 (PPO8) or a polypeptide with at least 75% sequence identity to SEQ ID NO: 47; SEQ ID NO: 48 (PPO9) or a polypeptide with at least 75% sequence identity to SEQ ID NO: 48;

and SEQ ID NO: 43 (PPO4) or a polypeptide with at least 75% sequence identity to SEQ ID NO: 43.

In particular embodiments of the DNA sequence, DNA construct, vector, plant cell transformed with a vector, banana plant cell transformed with a vector, and method of expressing a polyphenol oxidase in a plant cell, the PPO1 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151; the PPO2 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152; the PPO3 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153; the PPO4 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154; the PPO5 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155; the PPO6 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156; the PPO7 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157; the PPO8 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158; or the PPO9 polynucleotide comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159. In particular embodiments of the DNA sequence, DNA construct, vector, plant cell transformed with a vector, banana plant cell transformed with a vector, and method of expressing a polyphenol oxidase in a plant cell, the PPO1, PPO2, PPO8, PPO9, or PPO4 polyphenol oxidase polynucleotide comprises a polynucleotide sequence that is selected from the group consisting of SEQ ID NO: 151 (PPO1) or a polynucleotide with at least 75% sequence identity to SEQ ID NO: 151; SEQ ID NO: 152 (PPO2) or a polynucleotide with at least 75% sequence identity to SEQ ID NO: 152; SEQ ID NO: 158 (PPO8) or a polynucleotide with at least 75% sequence identity to SEQ ID NO: 158; SEQ ID NO: 159 (PPO9) or a polynucleotide with at least 75% sequence identity to SEQ ID NO: 159; and SEQ ID NO: 154 (PPO4) or a polynucleotide with at least 75% sequence identity to SEQ ID NO: 154.

Yet further provided by the invention is a synthetic banana polyphenol oxidase guide RNA comprising a variable region selected from the group consisting of: SEQ ID NOs: 32-39 and 57-97. Yet further provided by the invention is a synthetic banana polyphenol oxidase guide RNA comprising a variable region selected from the group consisting of: SEQ ID NOs: 180-196, 199-202 and 207-225. In some embodiments, the synthetic banana polyphenol oxidase guide RNA comprises a variable region selected from the group consisting of: SEQ ID NO: 32; SEQ ID NO: 33; SEQ ID NO: 34; SEQ ID NO:

35; SEQ ID NO: 36; SEQ ID NO: 37; SEQ ID NO: 57; SEQ ID NO: 58; SEQ ID NO: 38; SEQ ID NO: 39; SEQ ID NO: 62; SEQ ID NO: 76; and SEQ ID NO: 77.

The invention further provides a recombinant DNA construct comprising a promoter operably linked to a nucleotide sequence encoding at least one banana polyphenol oxidase guide RNA, wherein the guide RNA is capable of forming a complex with a CRISPR-associated endonuclease or modified CRISPR-associated endonuclease, and wherein the complex is capable of binding to and creating a double-strand or single-strand break in the at least one endogenous banana polyphenol oxidase gene. In some of these embodiments, the at least one endogenous polyphenol oxidase gene is selected from the group consisting of: PPO1, PPO2, PPO3, PPO4, PPO5, PPO6, PPO7, PPO8, and

PPO9. In some embodiments, the at least one endogenous polyphenol oxidase gene is a PPO1 gene. In some embodiments, the at least one endogenous polyphenol oxidase gene is a PPO2 gene. In some embodiments, the at least one endogenous polyphenol oxidase gene is a PPO3 gene. In some embodiments, the at least one endogenous polyphenol oxidase gene is a PPO4 gene. In some embodiments, the at least one endogenous polyphenol oxidase gene is a PPO5 gene. In some embodiments, the at least one endogenous polyphenol oxidase gene is a PPO6 gene. In some embodiments, the at least one endogenous polyphenol oxidase gene is a PPO7 gene. In some embodiments, the at least one endogenous polyphenol oxidase gene is a PPO8 gene. In some embodiments, the at least one endogenous polyphenol oxidase gene is a PPO9 gene. In some embodiments, the endogenous polyphenol oxidase genes are two or more of PPO1, PPO2, PPO3, PPO4, PPO5, PPO6, PPO7, PPO8, and PPO9 genes. In some embodiments, the endogenous polyphenol oxidase genes are PPO1 and PPO2 genes. In particular embodiments, the PPO1 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5. In particular embodiments, the PPO2 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6. In particular embodiments, the PPO3 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7. In particular embodiments, the PPO4 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8. In particular embodiments, the PPO5 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9. In particular embodiments, the PPO6 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10. In particular embodiments, the PPO7 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11. In particular embodiments, the PPO8 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12. In particular embodiments, the PPO9 gene comprises a coding sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13.

In particular embodiments, the PPO1, PPO2, PPO8, PPO9, or PPO4 polyphenol oxidase gene comprises a coding sequence that is selected from the group consisting of: SEQ ID NO: 5 (PPO1) or a polynucleotide with at least 75% sequence identity to SEQ ID NO: 5; SEQ ID NO: 6 (PPO2) or a polynucleotide with at least 75% sequence identity to SEQ ID NO: 6; SEQ ID NO: 12 (PPO8) or a polynucleotide with at least 75% sequence identity to SEQ ID NO: 12; SEQ ID NO: 13 (PPO9) or a polynucleotide with at least 75% sequence identity to SEQ ID NO: 13; and SEQ ID NO: 8 (PPO4) or a polynucleotide with at least 75% sequence identity to SEQ ID NO: 8. In particular embodiments, the PPO1 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40; the PPO2 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41; the PPO3 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42; the PPO4 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43; the PPO5 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44; the PPO6 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 45; the PPO7 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46; the PPO8 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 47; or the PPO9 gene encodes a polyphenol oxidase that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 48. In particular embodiments, the PPO1, PPO2, PPO8, PPO9, or PPO4 polyphenol oxidase gene encodes a polyphenol oxidase selected from the group consisting of: SEQ ID NO: 40 (PPO1) or a polypeptide with at least 75% sequence identity to SEQ ID NO: 40; SEQ ID NO: 41 (PPO2) or a polypeptide with at least 75% sequence identity to SEQ ID NO: 41; SEQ ID NO: 47 (PPO8) or a polypeptide with at least 75% sequence identity to SEQ ID NO: 47; SEQ ID NO: 48 (PPO9) or a polypeptide with at least 75% sequence identity to SEQ ID NO: 48; and SEQ ID NO: 43 (PPO4) or a polypeptide with at least 75% sequence identity to SEQ ID NO: 43. In particular embodiments, the PPO1 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 151. In particular embodiments, the PPO2 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 152. In particular embodiments, the PPO3 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 153. In particular embodiments, the PPO4 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 154. In particular embodiments, the PPO5 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 155. In particular embodiments, the PPO6 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 156. In particular embodiments, the PPO7 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157. In particular embodiments, the PPO8 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158. In particular embodiments, the PPO9 gene comprises a polynucleotide sequence that has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 159. In particular embodiments, the PPO1, PPO2, PPO8, PPO9, or PPO4 polyphenol oxidase gene comprises a polynucleotide sequence that is selected from the group consisting of:

SEQ ID NO: 151 (PPO1) or a polynucleotide with at least 75% sequence identity to SEQ ID NO: 151; SEQ ID NO: 152 (PPO2) or a polynucleotide with at least 75% sequence identity to SEQ ID NO: 152; SEQ ID NO: 158 (PPO8) or a polynucleotide with at least 75% sequence identity to SEQ ID NO: 158; SEQ ID NO: 159 (PPO9) or a polynucleotide with at least 75% sequence identity to SEQ ID NO: 159; and SEQ ID NO: 154 (PPO4) or a polynucleotide with at least 75% sequence identity to SEQ ID NO: 154.

In some of these embodiments, the one or more banana polyphenol oxidase guide RNAs comprises a variable region having a sequence selected from the group consisting of: SEQ ID NOs: 32-39 and 57-97; and any combination of the foregoing. In some embodiments the one or more banana polyphenol oxidase guide RNAs comprises a variable region having a sequence selected from the group consisting of: SEQ ID NOS:

180-196, 199-202 and 207-225; and any combination of the foregoing. In some embodiments, the synthetic banana polyphenol oxidase guide RNA comprises a variable region selected from the group consisting of: SEQ ID NO: 32; SEQ ID NO: 33; SEQ ID NO: 34; SEQ ID NO: 35; SEQ ID NO: 36; SEQ ID NO: 37; SEQ ID NO: 57; SEQ ID NO: 58; SEQ ID NO: 38; SEQ ID NO: 39; SEQ ID NO: 62; SEQ ID NO: 76; and SEQ ID NO: 77; and any combination of the foregoing. In some embodiments of the invention, said one or more guide RNAs are a pair of guide RNAs comprising variable regions selected from the group consisting of: SEQ ID NOs: 32 and 33; SEQ ID NOs: 34 and 35; SEQ ID NOs: 32 and 34; SEQ ID NOs: 32 and 35; SEQ ID NOs: 33 and 34; SEQ ID NOs: 33 and 35; SEQ ID NOs: 57 and 58; SEQ ID NOs: 38 and 39; SEQ ID NOs: 62 and 33; and SEQ ID NOs: 76 and 77. In some of these embodiments, the CRISPR-associated endonuclease is a Cas9 endonuclease.

Additional embodiments of the methods and compositions of the present invention are shown herein. Such embodiments include:
1. A method of reducing the level or activity of at least one endogenous PPO1, PPO2, PPO3, PPO4, PPO5, PPO6, PPO7, PPO8, or PPO9 polyphenol oxidase encoded by a PPO1, PPO2, PPO3, PPO4, PPO5, PPO6, PPO7, PPO8, or PPO9 polyphenol oxidase gene in a banana plant or banana plant cell.
2. The method of embodiment 1, wherein the PPO1, PPO2, PPO3, PPO4, PPO5, PPO6, PPO7, PPO8, or PPO9 polyphenol oxidase gene:
   (A) comprises a coding sequence selected from the group consisting of:
      (a) SEQ ID NO: 5 (PPO1) or a polynucleotide with at least 75% sequence identity to SEQ ID NO: 5;
      (b) SEQ ID NO: 6 (PPO2) or a polynucleotide with at least 75% sequence identity to SEQ ID NO: 6;
      (c) SEQ ID NO: 7 (PPO3) or a polynucleotide with at least 75% sequence identity to SEQ ID NO: 7;
      (d) SEQ ID NO: 8 (PPO4) or a polynucleotide with at least 75% sequence identity to SEQ ID NO: 8;
      (e) SEQ ID NO: 9 (PPO5) or a polynucleotide with at least 75% sequence identity to SEQ ID NO: 9;
      (f) SEQ ID NO: 10 (PPO6) or a polynucleotide with at least 75% sequence identity to SEQ ID NO: 10;
      (g) SEQ ID NO: 11 (PPO7) or a polynucleotide with at least 75% sequence identity to SEQ ID NO: 11;
      (h) SEQ ID NO: 12 (PPO8) or a polynucleotide with at least 75% sequence identity to SEQ ID NO: 12; and
      (i) SEQ ID NO: 13 (PPO9) or a polynucleotide with at least 75% sequence identity to SEQ ID NO: 13;
   (B) encodes a polyphenol oxidase selected from the group consisting of:
      (a) SEQ ID NO: 40 (PPO1) or a polypeptide with at least 75% sequence identity to SEQ ID NO: 40;
      (b) SEQ ID NO: 41 (PPO2) or a polypeptide with at least 75% sequence identity to SEQ ID NO: 41;
      (c) SEQ ID NO: 42 (PPO3) or a polypeptide with at least 75% sequence identity to SEQ ID NO: 42;
      (d) SEQ ID NO: 43 (PPO4) or a polypeptide with at least 75% sequence identity to SEQ ID NO: 43;
      (e) SEQ ID NO: 44 (PPO5) or a polypeptide with at least 75% sequence identity to SEQ ID NO: 44;
      (f) SEQ ID NO: 45 (PPO6) or a polypeptide with at least 75% sequence identity to SEQ ID NO: 45;
      (g) SEQ ID NO: 46 (PPO7) or a polypeptide with at least 75% sequence identity to SEQ ID NO: 46;
      (h) SEQ ID NO: 47 (PPO8) or a polypeptide with at least 75% sequence identity to SEQ ID NO: 47; and
      (i) SEQ ID NO: 48 (PPO9) or a polypeptide with at least 75% sequence identity to SEQ ID NO: 48; or
   (C) comprises a polynucleotide sequence selected from the group consisting of:
      (a) SEQ ID NO: 151 (PPO1) or a polynucleotide with at least 75% sequence identity to SEQ ID NO: 151;
      (b) SEQ ID NO: 152 (PPO2) or a polynucleotide with at least 75% sequence identity to SEQ ID NO: 152;
      (c) SEQ ID NO: 153 (PPO3) or a polynucleotide with at least 75% sequence identity to SEQ ID NO: 153;
      (d) SEQ ID NO: 154 (PPO4) or a polynucleotide with at least 75% sequence identity to SEQ ID NO: 154;
      (e) SEQ ID NO: 155 (PPO5) or a polynucleotide with at least 75% sequence identity to SEQ ID NO: 155;
      (f) SEQ ID NO: 156 (PPO6) or a polynucleotide with at least 75% sequence identity to SEQ ID NO: 156;
      (g) SEQ ID NO: 157 (PPO7) or a polynucleotide with at least 75% sequence identity to SEQ ID NO: 157;
      (h) SEQ ID NO: 158 (PPO8) or a polynucleotide with at least 75% sequence identity to SEQ ID NO: 158; and
      (i) SEQ ID NO: 159 (PPO9) or a polynucleotide with at least 75% sequence identity to SEQ ID NO: 159.
3. The method of embodiment 1 or 2, wherein the method results in:
   (a) a reduction in level of the at least one endogenous PPO1, PPO2, PPO3, PPO4, PPO5, PPO6, PPO7, PPO8, or PPO9 polyphenol oxidase in said banana plant or banana plant cell;
   (b) a reduction in function of the at least one endogenous PPO1, PPO2, PPO3, PPO4, PPO5, PPO6, PPO7, PPO8, or PPO9 polyphenol oxidase in said banana plant or banana plant cell; or
   (c) a loss of function of the at least one endogenous PPO1, PPO2, PPO3, PPO4, PPO5, PPO6, PPO7, PPO8, or PPO9 polyphenol oxidase in said banana plant or banana plant cell.
4. The method of any one of embodiments 1 to 3, wherein the method results in:
   (a) delayed browning of fruit flesh and/or fruit peel of said banana plant as compared to fruit flesh and/or fruit peel of a banana plant in which the level or activity of said at least one endogenous PPO1, PPO2, PPO3, PPO4, PPO5, PPO6, PPO7, PPO8, or PPO9 polyphenol oxidase is not reduced; and/or
   (b) reduced browning of fruit flesh and/or fruit peel of said banana plant as compared to fruit flesh and/or fruit peel of a banana plant in which the level or activity of said at least one endogenous PPO1, PPO2, PPO3, PPO4, PPO5, PPO6, PPO7, PPO8, or PPO9 polyphenol oxidase is not reduced.
5. The method of any one of embodiments 1 to 4, wherein the method comprises:
   (a) providing to said banana plant cell or to a part of said banana plant a silencing RNA targeting a transcript of said at least one PPO1, PPO2, PPO3, PPO4, PPO5, PPO6, PPO7, PPO8, or PPO9 polyphenol oxidase gene, optionally wherein providing said silencing RNA is by introducing an endonuclease to said banana plant cell or part of said banana plant, said endonuclease being capable of modifying a gene encoding an endogenous non-coding RNA such that it encodes said silencing RNA targeting a transcript of said at least one PPO1, PPO2, PPO3, PPO4, PPO5, PPO6, PPO7, PPO8, or PPO9 polyphenol oxidase gene, further optionally wherein the endonuclease is selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease; or
   (b) introducing a modification into the PPO1, PPO2, PPO3, PPO4, PPO5, PPO6, PPO7, PPO8, or PPO9 polyphenol oxidase gene encoding said at least one PPO1, PPO2, PPO3, PPO4, PPO5, PPO6, PPO7, PPO8, or PPO9 polyphenol oxidase; optionally wherein said modification is introduced into said the PPO1, PPO2, PPO3, PPO4, PPO5, PPO6, PPO7, PPO8, or PPO9 polyphenol oxidase gene by an endonuclease provided to said banana plant cell and capable of targeting said at least one PPO1, PPO2, PPO3, PPO4, PPO5, PPO6, PPO7, PPO8, or PPO9 polyphenol oxidase gene; further optionally wherein the endonuclease is selected from the group consisting of: a meganuclease, a zinc finger nuclease (ZFN), a transcription-activator like effector nuclease (TALEN), a homing endonuclease, a CRISPR-associated endonuclease, and a modified CRISPR-associated endonuclease; further optionally wherein said CRISPR-associated endonuclease is a Cas9 endonuclease.
6. The method of any one of embodiments 1 to 4, wherein the method comprises providing to said banana plant cell a CRISPR-associated endonuclease or a modified CRISPR-associated endonuclease, and one or more guide RNAs specific to said at least one PPO1, PPO2, PPO3, PPO4, PPO5, PPO6, PPO7, PPO8, or PPO9 polyphenol oxidase gene, wherein said CRISPR-associated endonuclease or modified CRISPR-associated endonuclease, and said one or more guide RNAS, form a complex that enables the CRISPR-associated endonuclease or modified CRISPR-associated endonuclease to introduce a double-strand or single-strand break at said at least one endogenous PPO1, PPO2, PPO3, PPO4, PPO5, PPO6, PPO7, PPO8, or PPO9 polyphenol oxidase gene; optionally wherein said CRISPR-associated endonuclease is a Cas9 endonuclease.
7. The method of embodiment 5 or 6, further comprising identifying at least one banana plant cell that comprises a modification of the at least one endogenous PPO1, PPO2, PPO3, PPO4, PPO5, PPO6, PPO7, PPO8, or PPO9 polyphenol oxidase gene, wherein the modification is selected from the group consisting of:
   (a) at least one nucleotide insertion;
   (b) at least one nucleotide deletion;
   (c) an insertion-deletion (indel);
   (d) an inversion;
   (e) at least one nucleotide substitution; and
   (f) any combination of (a) to (e).
8. The method of embodiment 6 or 7, wherein said one or more guide RNAs are provided to the banana plant cell within one or more recombinant DNA constructs encoding said one or more guide RNAs operably linked to one or more promoters.
9. The method of embodiment 6 or 7, wherein said CRISPR-associated endonuclease or modified CRISPR-associated endonuclease, and/or said one or more guide RNAs, are provided to said banana plant cell in RNA form.
10. The method of embodiment 6 or 7, wherein said CRISPR-associated endonuclease or modified CRISPR-associated endonuclease is provided to said banana plant cell in protein form and said one or more guide RNAs are provided to said banana plant cell in RNA form; optionally wherein said CRISPR-associated endonuclease or modified CRISPR-associated endonuclease and said one or more guide RNAs are provided to said banana plant cell as a ribonucleoprotein complex.
11. The method of any one of embodiments 5 to 10, wherein said endonuclease, said one or more guide RNAs and/or said one or more recombinant DNA constructs are provided to said banana plant cell using a method selected from the group consisting of:
   (a) particle bombardment;
   (b) Agrobacterium transformation;
   (c) protoplast transfection;
   (d) electroporation; and
   (e) nanoparticle-mediated transfection.
12. The method of any one of embodiments 5 to 11, wherein said endonuclease is provided to the banana plant cell as a polynucleotide encoding an endonuclease polypeptide; optionally wherein said endonuclease is Cas9 endonuclease and the polynucleotide is a Cas9 polynucleotide encoding a Cas9 polypeptide.
13. The method of embodiment 6 or 7, wherein said one or more guide RNAs and said CRISPR-associated endonuclease or modified CRISPR-associated endonuclease are provided to said banana plant cell via Agrobacterium transformation of one or more plasmids encoding said CRISPR-associated endonuclease or modified CRISPR-associated endonuclease, at least one selectable marker gene, and said one or more guide RNAs.
14. The method of any one of embodiments 6 to 13, wherein said one or more guide RNAs comprises a variable region having a sequence selected from the group consisting of: SEQ ID NOs: 32-39 and 57-97; and any combination of the foregoing, or wherein said one or more guide RNAs comprises a variable region having a sequence selected from the group consisting of: SEQ ID NOs: 180-196, 199-202 and 207-225; and any combination of the foregoing.
15. The method of any one of embodiments 6 to 13, wherein said one or more guide RNAs are a pair of guide RNAs comprising variable regions selected from the group consisting of:
   (a) SEQ ID NOs: 32 and 33;
   (b) SEQ ID NOs: 34 and 35;
   (c) SEQ ID NOs: 32 and 34;
   (d) SEQ ID NOs: 32 and 35;
   (e) SEQ ID NOs: 33 and 34;
   (f) SEQ ID NOs: 33 and 35;
   (g) SEQ ID NOs: 57 and 58;
   (h) SEQ ID NOs: 38 and 39;
   (i) SEQ ID NOs: 62 and 33; and
   (j) SEQ ID NOs: 76 and 77.
16. The method of any one of embodiments 1 to 15, wherein said banana plant cell is an embryogenic cell and/or contained in an embryogenic cell suspension.
17. A banana plant cell obtainable by the method of any one of embodiments 1 to 16.
18. The method of any one of embodiments 1 to 16, further comprising regenerating a banana plant from said banana plant cell;
   optionally further comprising harvesting fruit from said banana plant.
19. A banana plant or plant part obtainable by the method of embodiment 18; optionally wherein the banana plant or plant part comprises a mutated PPO1 gene:
   (a) as set forth in SEQ ID NO: 179;
   (b) that expresses a truncated PPO1 protein as set forth in SEQ ID NO: 177; or
   (c) that has a coding sequence as set forth in SEQ ID NO: 178;
   further optionally wherein the mutation is present in only one allele of the PPO1 gene.
20. Fruit harvested from a banana plant obtainable by the method of embodiment 18, wherein said fruit flesh and/or fruit peel is characterised by a phenotype of delayed and/or reduced browning as compared to fruit flesh and/or fruit peel of a banana plant in which the level or activity of said at least one endogenous PPO1, PPO2, PPO3, PPO4, PPO5, PPO6, PPO7, PPO8, or PPO9 polyphenol oxidase is not reduced.
21. A method of producing a banana plant characterised by a phenotype of delayed and/or reduced browning of fruit flesh and/or peel as compared to a wild-type banana plant, the method comprising:
   (a) providing to a banana plant cell a CRISPR-associated endonuclease or a modified CRISPR-associated endonuclease, and one or more guide RNAS, wherein said CRISPR-associated endonuclease or modified CRISPR-associated endonuclease and said one or more guide RNAs form a complex that enables the CRISPR-associated endonuclease or modified CRISPR-associated endonuclease to introduce a single-strand break or a double-strand break in at least one endogenous PPO1, PPO2, PPO3, PPO4, PPO5, PPO6, PPO7, PPO8, or PPO9 polyphenol oxidase gene:
   (A) comprising a coding sequence selected from the group consisting of:
      (i) SEQ ID NO: 5 or a polynucleotide with at least 75% sequence identity to SEQ ID NO: 5;
      (ii) SEQ ID NO: 6 or a polynucleotide with at least 75% sequence identity to SEQ ID NO: 6;
      (iii) SEQ ID NO: 7 or a polynucleotide with at least 75% sequence identity to SEQ ID NO: 7;
      (iv) SEQ ID NO: 8 or a polynucleotide with at least 75% sequence identity to SEQ ID NO: 8;
      (v) SEQ ID NO: 9 or a polynucleotide with at least 75% sequence identity to SEQ ID NO: 9;
      (vi) SEQ ID NO: 10 or a polynucleotide with at least 75% sequence identity to SEQ ID NO: 10;
      (vii) SEQ ID NO: 11 or a polynucleotide with at least 75% sequence identity to SEQ ID NO: 11;
      (viii) SEQ ID NO: 12 or a polynucleotide with at least 75% sequence identity to SEQ ID NO: 12; and
      (ix) SEQ ID NO: 13 or a polynucleotide with at least 75% sequence identity to SEQ ID NO: 13;
   (B) encoding a polyphenol oxidase selected from the group consisting of:
      (i) SEQ ID NO: 40 or a polypeptide with at least 75% sequence identity to SEQ ID NO: 40;
      (ii) SEQ ID NO: 41 or a polypeptide with at least 75% sequence identity to SEQ ID NO: 41;
      (iii) SEQ ID NO: 42 or a polypeptide with at least 75% sequence identity to SEQ ID NO: 42;
      (iv) SEQ ID NO: 43 or a polypeptide with at least 75% sequence identity to SEQ ID NO: 43;
      (v) SEQ ID NO: 44 or a polypeptide with at least 75% sequence identity to SEQ ID NO: 44;
      (vi) SEQ ID NO: 45 or a polypeptide with at least 75% sequence identity to SEQ ID NO: 45;
      (vii) SEQ ID NO: 46 or a polypeptide with at least 75% sequence identity to SEQ ID NO: 46;
      (viii) SEQ ID NO: 47 or a polypeptide with at least 75% sequence identity to SEQ ID NO: 47; and
      (ix) SEQ ID NO: 48 or a polypeptide with at least 75% sequence identity to SEQ ID NO: 48; or
   (C) comprising a polynucleotide sequence selected from the group consisting of:
      (i) SEQ ID NO: 151 or a polynucleotide with at least 75% sequence identity to SEQ ID NO: 151;
      (ii) SEQ ID NO: 152 or a polynucleotide with at least 75% sequence identity to SEQ ID NO: 152;
      (iii) SEQ ID NO: 153 or a polynucleotide with at least 75% sequence identity to SEQ ID NO: 153;
      (iv) SEQ ID NO: 154 or a polynucleotide with at least 75% sequence identity to SEQ ID NO: 154;
      (v) SEQ ID NO: 155 or a polynucleotide with at least 75% sequence identity to SEQ ID NO: 155;
      (vi) SEQ ID NO: 156 or a polynucleotide with at least 75% sequence identity to SEQ ID NO: 156;
      (vii) SEQ ID NO: 157 or a polynucleotide with at least 75% sequence identity to SEQ ID NO: 157;
      (viii) SEQ ID NO: 158 or a polynucleotide with at least 75% sequence identity to SEQ ID NO: 158; and
      (ix) SEQ ID NO: 159 or a polynucleotide with at least 75% sequence identity to SEQ ID NO: 159;
   (b) identifying at least one banana plant cell that comprises a modification of said at least one endogenous PPO1, PPO2, PPO3, PPO4, PPO5, PPO6, PPO7, PPO8, or PPO9 polyphenol oxidase gene, wherein the modification is selected from the group consisting of:
      (i) at least one nucleotide insertion;
      (ii) at least one nucleotide deletion;
      (iii) at least one nucleotide substitution; or
      (iv) any combination of (b)(i) to (b)(iii); and
   (c) regenerating a banana plant from said banana plant cell, wherein said banana plant is characterised by a phenotype of delayed and/or reduced browning of fruit flesh and/or peel as compared to a wild-type banana plant.
22. The method of embodiment 21, further comprising harvesting fruit from said banana plant, wherein said fruit is characterised by a phenotype of delayed and/or reduced browning as compared to fruit of a banana plant in which the level or activity of said at least one endogenous PPO1, PPO2, PPO3, PPO4, PPO5, PPO6, PPO7, PPO8, or PPO9 polyphenol oxidase is not reduced or lost.
23. A banana plant or plant part comprising in its genome at least one modified endogenous PPO1, PPO2, PPO3, PPO4, PPO5, PPO6, PPO7, PPO8, or PPO9 polyphenol oxidase gene, wherein said modification results in a reduction in, or loss of function of, the at least one endogenous PPO1, PPO2, PPO3, PPO4, PPO5, PPO6, PPO7, PPO8, or PPO9 polyphenol oxidase encoded by said modified endogenous PPO1, PPO2, PPO3, PPO4, PPO5, PPO6, PPO7, PPO8, or PPO9 polyphenol oxidase gene, wherein said modification is located in at least one endogenous PPO1, PPO2, PPO3, PPO4, PPO5, PPO6, PPO7, PPO8, or PPO9 polyphenol oxidase gene:
   (A) comprising a coding sequence selected from the group consisting of:
      (a) SEQ ID NO: 5 or a polynucleotide with at least 75% sequence identity to SEQ ID NO: 5;
      (b) SEQ ID NO: 6 or a polynucleotide with at least 75% sequence identity to SEQ ID NO: 6;
      (c) SEQ ID NO: 7 or a polynucleotide with at least 75% sequence identity to SEQ ID NO: 7;
      (d) SEQ ID NO: 8 or a polynucleotide with at least 75% sequence identity to SEQ ID NO: 8;
      (e) SEQ ID NO: 9 or a polynucleotide with at least 75% sequence identity to SEQ ID NO: 9;
      (f) SEQ ID NO: 10 or a polynucleotide with at least 75% sequence identity to SEQ ID NO: 10;
      (g) SEQ ID NO: 11 or a polynucleotide with at least 75% sequence identity to SEQ ID NO: 11;
      (h) SEQ ID NO: 12 or a polynucleotide with at least 75% sequence identity to SEQ ID NO: 12; and
      (i) SEQ ID NO: 13 or a polynucleotide with at least 75% sequence identity to SEQ ID NO: 13;
   (B) encoding a polyphenol oxidase selected from the group consisting of:
      (a) SEQ ID NO: 40 or a polypeptide with at least 75% sequence identity to SEQ ID NO: 40;
      (b) SEQ ID NO: 41 or a polypeptide with at least 75% sequence identity to SEQ ID NO: 41;
      (c) SEQ ID NO: 42 or a polypeptide with at least 75% sequence identity to SEQ ID NO: 42;
      (d) SEQ ID NO: 43 or a polypeptide with at least 75% sequence identity to SEQ ID NO: 43;
      (e) SEQ ID NO: 44 or a polypeptide with at least 75% sequence identity to SEQ ID NO: 44;
      (f) SEQ ID NO: 45 or a polypeptide with at least 75% sequence identity to SEQ ID NO: 45;
      (g) SEQ ID NO: 46 or a polypeptide with at least 75% sequence identity to SEQ ID NO: 46;
      (h) SEQ ID NO: 47 or a polypeptide with at least 75% sequence identity to SEQ ID NO: 47; and
      (i) SEQ ID NO: 48 or a polypeptide with at least 75% sequence identity to SEQ ID NO: 48; or
   (C) comprising a polynucleotide sequence selected from the group consisting of:
      (a) SEQ ID NO: 151 or a polynucleotide with at least 75% sequence identity to SEQ ID NO: 151;
      (b) SEQ ID NO: 152 or a polynucleotide with at least 75% sequence identity to SEQ ID NO: 152;
      (c) SEQ ID NO: 153 or a polynucleotide with at least 75% sequence identity to SEQ ID NO: 153;
      (d) SEQ ID NO: 154 or a polynucleotide with at least 75% sequence identity to SEQ ID NO: 154;
      (e) SEQ ID NO: 155 or a polynucleotide with at least 75% sequence identity to SEQ ID NO: 155;
      (f) SEQ ID NO: 156 or a polynucleotide with at least 75% sequence identity to SEQ ID NO: 156;
      (g) SEQ ID NO: 157 or a polynucleotide with at least 75% sequence identity to SEQ ID NO: 157;
      (h) SEQ ID NO: 158 or a polynucleotide with at least 75% sequence identity to SEQ ID NO: 158; and
      (i) SEQ ID NO: 159 or a polynucleotide with at least 75% sequence identity to SEQ ID NO: 159.
24. The banana plant or plant part of embodiment 19 or 23, wherein the banana plant or plant part is non-transgenic.
25. Banana fruit harvested from the banana plant of embodiment 23 or 24, wherein said fruit is characterised by a phenotype of delayed and/or reduced browning as compared to fruit of a banana plant in which the level or activity of said at least one endogenous PPO1, PPO2, PPO3, PPO4, PPO5, PPO6, PPO7, PPO8, or PPO9 polyphenol oxidase is not reduced.
26. A method of obtaining a banana fruit food product, the method comprising processing the banana fruit of embodiment 25.
27. A DNA sequence comprising a banana PPO1, PPO2, PPO3, PPO4, PPO5, PPO6, PPO7, PPO8, or PPO9 polyphenol oxidase polynucleotide:
   (A) comprising a coding sequence selected from the group consisting of:
      (a) SEQ ID NO: 5 or a polynucleotide with at least 75% sequence identity to SEQ ID NO: 5;
      (b) SEQ ID NO: 6 or a polynucleotide with at least 75% sequence identity to SEQ ID NO: 6;
      (c) SEQ ID NO: 7 or a polynucleotide with at least 75% sequence identity to SEQ ID NO: 7;
      (d) SEQ ID NO: 8 or a polynucleotide with at least 75% sequence identity to SEQ ID NO: 8;
      (e) SEQ ID NO: 9 or a polynucleotide with at least 75% sequence identity to SEQ ID NO: 9;
      (f) SEQ ID NO: 10 or a polynucleotide with at least 75% sequence identity to SEQ ID NO: 10;
      (g) SEQ ID NO: 11 or a polynucleotide with at least 75% sequence identity to SEQ ID NO: 11;
      (h) SEQ ID NO: 12 or a polynucleotide with at least 75% sequence identity to SEQ ID NO: 12; and
      (i) SEQ ID NO: 13 or a polynucleotide with at least 75% sequence identity to SEQ ID NO: 13;
   (B) encoding a polyphenol oxidase selected from the group consisting of:
      (a) SEQ ID NO: 40 or a polypeptide with at least 75% sequence identity to SEQ ID NO: 40;
      (b) SEQ ID NO: 41 or a polypeptide with at least 75% sequence identity to SEQ ID NO: 41;
      (c) SEQ ID NO: 42 or a polypeptide with at least 75% sequence identity to SEQ ID NO: 42;
      (d) SEQ ID NO: 43 or a polypeptide with at least 75% sequence identity to SEQ ID NO: 43;
      (e) SEQ ID NO: 44 or a polypeptide with at least 75% sequence identity to SEQ ID NO: 44;
      (f) SEQ ID NO: 45 or a polypeptide with at least 75% sequence identity to SEQ ID NO: 45;
      (g) SEQ ID NO: 46 or a polypeptide with at least 75% sequence identity to SEQ ID NO: 46;
      (h) SEQ ID NO: 47 or a polypeptide with at least 75% sequence identity to SEQ ID NO: 47; and
      (i) SEQ ID NO: 48 or a polypeptide with at least 75% sequence identity to SEQ ID NO: 48; or
   (C) comprising a polynucleotide sequence selected from the group consisting of:
      (a) SEQ ID NO: 151 or a polynucleotide with at least 75% sequence identity to SEQ ID NO: 151;
      (b) SEQ ID NO: 152 or a polynucleotide with at least 75% sequence identity to SEQ ID NO: 152;
      (c) SEQ ID NO: 153 or a polynucleotide with at least 75% sequence identity to SEQ ID NO: 153;
      (d) SEQ ID NO: 154 or a polynucleotide with at least 75% sequence identity to SEQ ID NO: 154;
      (e) SEQ ID NO: 155 or a polynucleotide with at least 75% sequence identity to SEQ ID NO: 155;
      (f) SEQ ID NO: 156 or a polynucleotide with at least 75% sequence identity to SEQ ID NO: 156;
      (g) SEQ ID NO: 157 or a polynucleotide with at least 75% sequence identity to SEQ ID NO: 157;
      (h) SEQ ID NO: 158 or a polynucleotide with at least 75% sequence identity to SEQ ID NO: 158; and
      (i) SEQ ID NO: 159 or a polynucleotide with at least 75% sequence identity to SEQ ID NO: 159.
28. A DNA construct or vector comprising a banana PPO1, PPO2, PPO3, PPO4, PPO5, PPO6, PPO7, PPO8, or PPO9 polyphenol oxidase polynucleotide:
   (A) comprising a coding sequence selected from the group consisting of:
      (a) SEQ ID NO: 5 or a polynucleotide with at least 75% sequence identity to SEQ ID NO: 5;
      (b) SEQ ID NO: 6 or a polynucleotide with at least 75% sequence identity to SEQ ID NO: 6;
      (c) SEQ ID NO: 7 or a polynucleotide with at least 75% sequence identity to SEQ ID NO: 7;
      (d) SEQ ID NO: 8 or a polynucleotide with at least 75% sequence identity to SEQ ID NO: 8;
      (e) SEQ ID NO: 9 or a polynucleotide with at least 75% sequence identity to SEQ ID NO: 9;
      (f) SEQ ID NO: 10 or a polynucleotide with at least 75% sequence identity to SEQ ID NO: 10;
      (g) SEQ ID NO: 11 or a polynucleotide with at least 75% sequence identity to SEQ ID NO: 11;
      (h) SEQ ID NO: 12 or a polynucleotide with at least 75% sequence identity to SEQ ID NO: 12; and
      (i) SEQ ID NO: 13 or a polynucleotide with at least 75% sequence identity to SEQ ID NO: 13;
   (B) encoding a polyphenol oxidase selected from the group consisting of:
      (a) SEQ ID NO: 40 or a polypeptide with at least 75% sequence identity to SEQ ID NO: 40;
      (b) SEQ ID NO: 41 or a polypeptide with at least 75% sequence identity to SEQ ID NO: 41;
      (c) SEQ ID NO: 42 or a polypeptide with at least 75% sequence identity to SEQ ID NO: 42;
      (d) SEQ ID NO: 43 or a polypeptide with at least 75% sequence identity to SEQ ID NO: 43;
      (e) SEQ ID NO: 44 or a polypeptide with at least 75% sequence identity to SEQ ID NO: 44;
      (f) SEQ ID NO: 45 or a polypeptide with at least 75% sequence identity to SEQ ID NO: 45;
      (g) SEQ ID NO: 46 or a polypeptide with at least 75% sequence identity to SEQ ID NO: 46;
      (h) SEQ ID NO: 47 or a polypeptide with at least 75% sequence identity to SEQ ID NO: 47; and
      (i) SEQ ID NO: 48 or a polypeptide with at least 75% sequence identity to SEQ ID NO: 48; or
   (C) comprising a polynucleotide sequence selected from the group consisting of:
      (a) SEQ ID NO: 151 or a polynucleotide with at least 75% sequence identity to SEQ ID NO: 151;
      (b) SEQ ID NO: 152 or a polynucleotide with at least 75% sequence identity to SEQ ID NO: 152;
      (c) SEQ ID NO: 153 or a polynucleotide with at least 75% sequence identity to SEQ ID NO: 153;
      (d) SEQ ID NO: 154 or a polynucleotide with at least 75% sequence identity to SEQ ID NO: 154;
      (e) SEQ ID NO: 155 or a polynucleotide with at least 75% sequence identity to SEQ ID NO: 155;
      (f) SEQ ID NO: 156 or a polynucleotide with at least 75% sequence identity to SEQ ID NO: 156;
      (g) SEQ ID NO: 157 or a polynucleotide with at least 75% sequence identity to SEQ ID NO: 157;
      (h) SEQ ID NO: 158 or a polynucleotide with at least 75% sequence identity to SEQ ID NO: 158; and
      (i) SEQ ID NO: 159 or a polynucleotide with at least 75% sequence identity to SEQ ID NO: 159.
29. A plant cell transformed with the vector or embodiment 28, optionally wherein the plant cell is a banana plant cell.
30. A polyphenol oxidase protein:
   (a) encoded by any one of SEQ ID NOs: 5 to 13, or encoded by a polynucleotide with at least 75% sequence identity to any one of SEQ ID NOs: 5 to 13;
   (b) comprising any one of SEQ ID NOs: 40 to 48, or comprising a sequence with at least 75% sequence identity to any one of SEQ ID NOs: 40 to 48; or
   (c) encoded by any one of SEQ ID NOs: 151 to 159, or encoded by a polynucleotide with at least 75% sequence identity to any one of SEQ ID NOs: 151 to 159.
31. A method of expressing a polyphenol oxidase in a plant cell, wherein the method comprises introducing into said plant cell a banana polyphenol oxidase polynucleotide:
   (A) comprising a coding sequence selected from the group consisting of:
      (a) SEQ ID NO: 5 or a polynucleotide with at least 75% sequence identity to SEQ ID NO: 5;
      (b) SEQ ID NO: 6 or a polynucleotide with at least 75% sequence identity to SEQ ID NO: 6;
      (c) SEQ ID NO: 7 or a polynucleotide with at least 75% sequence identity to SEQ ID NO: 7;
      (d) SEQ ID NO: 8 or a polynucleotide with at least 75% sequence identity to SEQ ID NO: 8;
      (e) SEQ ID NO: 9 or a polynucleotide with at least 75% sequence identity to SEQ ID NO: 9;
      (f) SEQ ID NO: 10 or a polynucleotide with at least 75% sequence identity to SEQ ID NO: 10;
      (g) SEQ ID NO: 11 or a polynucleotide with at least 75% sequence identity to SEQ ID NO: 11;
      (h) SEQ ID NO: 12 or a polynucleotide with at least 75% sequence identity to SEQ ID NO: 12; and
      (i) SEQ ID NO: 13 or a polynucleotide with at least 75% sequence identity to SEQ ID NO: 13;
   (B) encoding a polyphenol oxidase selected from the group consisting of:
      (a) SEQ ID NO: 40 or a polypeptide with at least 75% sequence identity to SEQ ID NO: 40;
      (b) SEQ ID NO: 41 or a polypeptide with at least 75% sequence identity to SEQ ID NO: 41;
      (c) SEQ ID NO: 42 or a polypeptide with at least 75% sequence identity to SEQ ID NO: 42;
      (d) SEQ ID NO: 43 or a polypeptide with at least 75% sequence identity to SEQ ID NO: 43;
      (e) SEQ ID NO: 44 or a polypeptide with at least 75% sequence identity to SEQ ID NO: 44;
      (f) SEQ ID NO: 45 or a polypeptide with at least 75% sequence identity to SEQ ID NO: 45;
      (g) SEQ ID NO: 46 or a polypeptide with at least 75% sequence identity to SEQ ID NO: 46;
      (h) SEQ ID NO: 47 or a polypeptide with at least 75% sequence identity to SEQ ID NO: 47; and
      (i) SEQ ID NO: 48 or a polypeptide with at least 75% sequence identity to SEQ ID NO: 48; or
   (C) comprising a polynucleotide sequence selected from the group consisting of:
      (a) SEQ ID NO: 151 or a polynucleotide with at least 75% sequence identity to SEQ ID NO: 151;
      (b) SEQ ID NO: 152 or a polynucleotide with at least 75% sequence identity to SEQ ID NO: 152;
      (c) SEQ ID NO: 153 or a polynucleotide with at least 75% sequence
      (d) SEQ ID NO: 154 or a polynucleotide with at least 75% sequence identity to SEQ ID NO: 154;
      (e) SEQ ID NO: 155 or a polynucleotide with at least 75% sequence identity to SEQ ID NO: 155;
      (f) SEQ ID NO: 156 or a polynucleotide with at least 75% sequence identity to SEQ ID NO: 156;
      (g) SEQ ID NO: 157 or a polynucleotide with at least 75% sequence identity to SEQ ID NO: 157;
      (h) SEQ ID NO: 158 or a polynucleotide with at least 75% sequence identity to SEQ ID NO: 158; and
      (i) SEQ ID NO: 159 or a polynucleotide with at least 75% sequence identity to SEQ ID NO: 159;
      operably linked to a promoter active in a plant cell.
32. A synthetic banana polyphenol oxidase guide RNA comprising a variable region selected from the group consisting of: SEQ ID NOs: 32-39 and 57-97, or selected from the group consisting of SEQ ID NOs: 180-196, 199-202 and 207-225.
33. A recombinant DNA construct comprising a promoter operably linked to a nucleotide sequence expressing at least one banana PPO1, PPO2, PPO3, PPO4, PPO5, PPO6, PPO7, PPO8, or PPO9 polyphenol oxidase guide RNA, wherein said guide RNA is capable of forming a complex with a CRISPR-associated endonuclease or modified CRISPR-associated endonuclease, and wherein said complex is capable of binding to and creating a double-strand or single-strand break in the at least one endogenous banana PPO1, PPO2, PPO3, PPO4, PPO5, PPO6, PPO7, PPO8, or PPO9 polyphenol oxidase gene:
   (A) comprising a coding sequence selected from the group consisting of:
      (a) SEQ ID NO: 5 or a polynucleotide with at least 75% sequence identity to SEQ ID NO: 5;
      (b) SEQ ID NO: 6 or a polynucleotide with at least 75% sequence identity to SEQ ID NO: 6;
      (c) SEQ ID NO: 7 or a polynucleotide with at least 75% sequence identity to SEQ ID NO: 7;
      (d) SEQ ID NO: 8 or a polynucleotide with at least 75% sequence identity to SEQ ID NO: 8;
      (e) SEQ ID NO: 9 or a polynucleotide with at least 75% sequence identity to SEQ ID NO: 9;
      (f) SEQ ID NO: 10 or a polynucleotide with at least 75% sequence identity to SEQ ID NO: 10;
      (g) SEQ ID NO: 11 or a polynucleotide with at least 75% sequence identity to SEQ ID NO: 11;
      (h) SEQ ID NO: 12 or a polynucleotide with at least 75% sequence identity to SEQ ID NO: 12; and
      (i) SEQ ID NO: 13 or a polynucleotide with at least 75% sequence identity to SEQ ID NO: 13;
   (B) encoding a polyphenol oxidase selected from the group consisting of:
      (a) SEQ ID NO: 40 or a polypeptide with at least 75% sequence identity to SEQ ID NO: 40;
      (b) SEQ ID NO: 41 or a polypeptide with at least 75% sequence identity to SEQ ID NO: 41;
      (c) SEQ ID NO: 42 or a polypeptide with at least 75% sequence identity to SEQ ID NO: 42;
      (d) SEQ ID NO: 43 or a polypeptide with at least 75% sequence identity to SEQ ID NO: 43;
      (e) SEQ ID NO: 44 or a polypeptide with at least 75% sequence identity to SEQ ID NO: 44;
      (f) SEQ ID NO: 45 or a polypeptide with at least 75% sequence identity to SEQ ID NO: 45;
      (g) SEQ ID NO: 46 or a polypeptide with at least 75% sequence identity to SEQ ID NO: 46;
      (h) SEQ ID NO: 47 or a polypeptide with at least 75% sequence identity to SEQ ID NO: 47; and
      (i) SEQ ID NO: 48 or a polypeptide with at least 75% sequence identity to SEQ ID NO: 48; or
   (C) comprising a polynucleotide sequence selected from the group consisting of:
      (a) SEQ ID NO: 151 or a polynucleotide with at least 75% sequence identity to SEQ ID NO: 151;
      (b) SEQ ID NO: 152 or a polynucleotide with at least 75% sequence identity to SEQ ID NO: 152;
      (c) SEQ ID NO: 153 or a polynucleotide with at least 75% sequence
      (d) SEQ ID NO: 154 or a polynucleotide with at least 75% sequence identity to SEQ ID NO: 154;
      (e) SEQ ID NO: 155 or a polynucleotide with at least 75% sequence identity to SEQ ID NO: 155;
      (f) SEQ ID NO: 156 or a polynucleotide with at least 75% sequence identity to SEQ ID NO: 156;
      (g) SEQ ID NO: 157 or a polynucleotide with at least 75% sequence identity to SEQ ID NO: 157;
      (h) SEQ ID NO: 158 or a polynucleotide with at least 75% sequence identity to SEQ ID NO: 158; and
      (i) SEQ ID NO: 159 or a polynucleotide with at least 75% sequence identity to SEQ ID NO: 159;
      in the banana genome.
34. The recombinant DNA construct of embodiment 33, wherein the one or more banana polyphenol oxidase guide RNAs comprises a variable region having a sequence selected from the group consisting of: SEQ ID NOs: 32-39 and 57-97; and any combination of the foregoing, or wherein the one or more banana polyphenol oxidase guide RNAs comprises a variable region having a sequence selected from the group consisting of: SEQ ID NOs: 180-196, 199-202 and 207-225; and any combination of the foregoing.
35. A recombinant DNA construct of embodiment 33 or 34, wherein said CRISPR-associated endonuclease is a Cas9 endonuclease.

Unless otherwise defined, all technical and/or scientific terms used herein have the same meaning as commonly understood by one of ordinary skill in the art to which the invention pertains. Although methods and materials similar or equivalent to those described herein can be used, exemplary methods and/or materials are described. The materials, methods, and examples are illustrative only and are not intended to be limiting.

The terms “comprises”, “comprising”, “includes”, “including”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an”, and “the” include plural references unless the context dictates otherwise. For example, the term “a compound” or “at least one compound” may include a plurality of compounds, including mixtures thereof. As used herein the term “about” refers to +/−10%.

### EXAMPLES

The following examples are illustrative and not considered to limit the scope of the invention.

The nomenclature and laboratory procedures used herein include molecular, biochemical, microbiological and recombinant DNA techniques. Such techniques are explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout. The procedures therein are well known in the art and are provided for the convenience of the reader. All the information contained therein is incorporated herein by reference.

### Example 1—Measurement of Browning in Banana

Browning can be measured based on peel colour, in which case a browning index is constructed such that a visual assessment of the colour of the peel is correlated to the number of days it takes banana fruits to reach such a colour after flower appearance (see “Dole Retail Banana Ripening Guide” https://www.dolenz.co.nz/uploads/media/59236082048a9/banana-trade-section-web.pdf and Gooding et al., “Molecular cloning and characterisation of banana fruit polyphenol oxidase”, Planta, September 2001; 123(5): 748-57). This is based on the fact that fruits mature in about 60 to 90 days after flower appearance. In order to build the index, banana fruits are collected as early as 40 days (stage 1) up until they turn very brown (stage 10), and each colour is correlated to the number of days following flower appearance. In the index, stage 10 is expected to be reached around 90 days after flower appearance. The fruit colour is visually assessed at all 10 stages based on the description of colour defined for example in the “Dole Retail Banana Ripening Guide”. Stage 1 is when all fingers of the bunch have green peel (40 days after flower appearance), stage 2 is when there is a first colour change to light green, usually seen in the shoulders (45 days after flower appearance), stage 3 is when bananas are more green than yellow (50 days after flower appearance), stage 4 is when bananas are more yellow than green (60 days after flower appearance), stage 5 is when bananas are yellow with green tips (65 days after flower appearance), and stage 6 is when bananas are full yellow (70 days after flower appearance), while at stage 7, all fingers of the bunch are yellow-flecked with brown peel (75 days after flower appearance), at stage 8, progressive browning has occurred (80 days after flower appearance), at stage 9, still further browning has occurred (85 days after flower appearance), and at stage 10, all fingers of the bunch have dark-brown peel (90 days after flower appearance). To standardise the measure of colour, colorimetric coordinates are taken with a Minolta Chroma Meter CR 400 or a Minolta CR-300 Chroma Meter with DP-301 Data Processor. The measuring head of the CR-300 uses diffuse illumination/0° viewing geometry (specular component included) to provide measurements of a wide variety of surfaces which correlate well with colour, as seen under diffuse lighting conditions, as is described in Bruno Bonnet, C., Hubert, O., Mbeguie-A-Mbeguie, D. et al., Effect of physiological harvest stages on the composition of bioactive compounds in Cavendish bananas. J. Zhejiang Univ. Sci. B 14, 270-278 (2013). This allows measuring reflected colour at each stage of fruit development (1 to 10), and these data are to correlate peel colour to fruit ripening and browning.

Browning can also be measured based on a banana browning guide that makes use of a visual assessment of sliced and pureed banana pulp (flesh) over time (from 0 to 180 hours). Three fingers are collected from banana bunches representing stages 3 to 10 (see above), and the peel is gently (to avoid mechanical damage that may lead to browning) washed for 5 minutes in 0.2% sodium hypochlorite. Next, banana puree is prepared from each individual banana finger after peeling, cutting into pieces, and homogenization in an electrical blender or food processor. The resulting puree is poured on Petri dishes, and images are captured at 0, 15, 30, 60, and 120 minutes and later at 24, 48, and 72 hours. In addition, banana slices are cut and placed on Petri dishes, and images are captured at 0, 12, 24, 36, 48, and 72 hours for banana bunches at colour stage 3 and 4. For banana fingers at stages 5 up to 10, images of banana slices are captured every 8 hours from 0 to 180 hours (˜22 time points). This measurement method is based on results in Chi et al. (2014) (Chi, M., Bhagwat, B., Lane, W. D. et al. Reduced polyphenol oxidase gene expression and enzymatic browning in potato (Solanum tuberosum L.) with artificial microRNAs. BMC Plant Biol 14, 62 (2014). https://doi.org/10.1186/1471-2229-14-62) and Escalante-Minakata, P., Ibarra-Junquera, V., Ornelas-Paz, J.d. et al., Comparative study of the banana pulp browning process of ‘Giant Dwarf’ and FHIA-23 during fruit ripening based on image analysis and the polyphenol oxidase and peroxidase biochemical properties. 3 Biotech 8, 30 (2018). Images are processed to correlate image colour to browning. Colour is expected to vary somewhere from freshly cut banana slices or banana puree colour (yellow/off-white) to brown bananas (dark brown).

Browning can also be measured based on an assessment of pulp firmness and peel hardness. Pulp firmness and peel hardness are measured with a TA-XT2 penetrometer as described in Bruno Bonnet, C., Hubert, O., Mbeguie-A-Mbeguie, D. et al., Effect of physiological harvest stages on the composition of bioactive compounds in Cavendish bananas. J. Zhejiang Univ. Sci. B 14, 270-278 (2013). Three banana fingers are taken from bunches representing stages 3 to 10 (as established based on peel colour; see above). Using a cylindrical 4.9 mm metal borer, the clean, fresh, unpeeled fruits are penetrated at a constant speed (2 mm/s) to a depth of 10 mm. The maximum force applied to break the peel represents the peel hardness, and the slope of the force/time curve represents the fruit firmness.

Browning can also be measured based on a correlation of peel colour (and firmness) with flesh colour/texture. This make use of a catalogue of colour (visual and colorimetric measurements), peel hardness, and pulp firmness vs banana maturation stages and browning of peel and flesh relative to time in wild type plants. This creates the standard by which a reduction of browning can be assessed in banana peel and flesh.

### Example 2—Identification of PPOs in Banana

In order to identify the banana PPO genes, a bioinformatic analysis was performed. PPO peptide sequences from the arctic apple as provided in U.S. Pat. No. 9,580,723 (SEQ ID NOs: 1 to 4) were used as query sequences to search for homologues in the banana genome and genomes of further plant species known to have a PPO gene (apricot, sweet potato, pokeweed, tobacco, tomato, potato and grape). The “TBlastN” tool was used to align the query protein sequences with nucleotide sequences of the chosen genomes which have been translated. As a result, 72 sequences from various species homologous to the query sequence were found, including nine from banana (Musa acuminata).

The resulting sequences were subjected to Multiple Sequence Alignment (MSA), following which a phylogenetic tree was constructed. The genes were arranged in clusters. Each cluster was assigned confidence scoring by combining the Maximum Likeliness and the Neighbour Joining algorithms. The rationale behind this is that banana genes that cluster with known PPO genes with a high degree of confidence are more likely to also have conserved PPO activity. The analysis showed that all nine banana genes identified by “TBlastN” cluster with PPO genes from other species with a high degree of confidence. Thus, all nine genes were predicted to be PPO genes with conserved functionality. The analysis and the retrieved PPOs were confirmed by running additional phylogeny analyses. These PPOs have between 35% to 97% homology to each other, as shown in FIG. 1 and between about 39% to 97% homology to the query sequences as shown in FIG. 2. The identified banana PPOs were further confirmed by aligning their DWL domain with that of the query sequences, as can be seen in FIG. 3. The nine PPO banana peptide homologues, along with their corresponding accession numbers from the Banana Genome Hub, are listed in Table 1 and set forth in SEQ ID NOs: 40 to 48 (the corresponding gene sequences listed in SEQ ID NOs: 5 to 13).

TABLE 1
Identified PPOs from Musa acuminata

| Potential PPO | SEQ ID NO: | Accession number (Banana Genome Hub) |
|---|---|---|
| PPO1 | 40 | Ma06_31080 |
| PPO2 | 41 | Ma07_03540 |
| PPO3 | 42 | Ma07_03650 |
| PPO4 | 43 | Ma08_09150 |
| PPO5 | 44 | Ma08_09160 |
| PPO6 | 45 | Ma08_09170 |
| PPO7 | 46 | Ma08_09180 |
| PPO8 | 47 | Ma08_34740 |
| PPO9 | 48 | Ma10_20510 |

### Example 3—Selection of PPO Candidates for Targeting

In order to determine the banana tissues in which the PPOs of Table 1 are expressed, mRNA from various banana tissues was produced, in particular from embryonic cell suspension (ECS), embryos which differentiated from ECS following incubation in embryo development medium (EDM), rolled leaf, top leaf, old leaf, brown peel, yellow peel, green peel, brown fruit, yellow fruit, and green fruit. RNA extraction was carried out by snap freezing banana tissues in liquid nitrogen, freeze-drying, and homogenising. The samples were then placed in tubes with extraction buffer and allowed to defrost while mixing. The samples were centrifuged, and the supernatant transferred to a fresh tube. Phenol-chloroform extraction was then used, followed by centrifugation to achieve an RNA pellet. The extraction of mRNA was then completed using the Plant/Fungi Total RNA Purification Kit (Norgen Biotek Corp).

The expression of mRNAs encoding the nine identified PPOs was examined in all the above tissues and using semi-quantitative PCR, with three to six biological repeats for each tissue/PPO combination. DNA was removed from the RNA samples using the Turbo DNA-free kit (Invitrogen). DNAse-treated RNA was used to synthesise cDNA using Superscript III (Thermofisher), with a mixture of oligo-dT and random hexamers to ensure complete coverage of transcripts. For each PCR, 12 ng of cDNA was used as template, and 30 cycles of a standard PCR reaction were performed using GoTaq G2 master mix (Promega). The oligonucleotides used in the PCR reactions are shown in Table 2 (and in SEQ ID NOs: 14 to 31):

TABLE 2
Primers used to identify the expression of mRNA-encoding PPOs.

| | | SEQ | | SEQ | Annealing | |
|---|---|---|---|---|---|---|
| | | ID | Reverse | ID | Temperature | Amplicon |
| PPO | Forward primer | NO: | primer | NO: | (° C.) | size |
| PPO1 | CAGCTTCGCGAT | 14 | TTCCAGATGCG | 15 | 53.2 | 438 |
| | TCCGTTCT | | GTCGATGTT | | | (cDNA) |
| | | | | | | 748 |
| | | | | | | (gDNA) |
| PPO2 | GAGCACTCCATG | 16 | GGAAGCCGAT | 17 | 53.9 | 459 |
| | TTCGTCCC | | CTGGTCGTAA | | | (cDNA) |
| | | | | | | 459 |
| | | | | | | (gDNA) |
| PPO3 | AAGTTTCTGCGA | 18 | CAGTTCCAGAA | 19 | 53.3 | 420 |
| | CCCCAAGA | | AGGGAGCGT | | | (cDNA) |
| | | | | | | 420 |
| | | | | | | (gDNA) |
| PPO4 | GAGTTCGAAGA | 20 | GTCGCTTCCCT | 21 | 52.3 | 477 |
| | CAACGACTGG | | TCATCCTATGC | | | (cDNA) |
| | | | | | | 562 |
| | | | | | | (gDNA) |
| PPO5 | GAGCCGAGCGG | 22 | GTCTCGTTGG | 23 | 53.8 | 494 |
| | AAACTATGA | | TGGTCACCTT | | | (cDNA) |
| | | | | | | 579 |
| | | | | | | (gDNA) |
| PPO6 | ACCGAAAACACT | 24 | CGGGTTAAGG | 25 | 53.6 | 414 |
| | GCATCCGA | | CAGTCCTGG | | | (cDNA) |
| | | | | | | 499 |
| | | | | | | (gDNA) |
| PPO7 | AAAACCCTGCCT | 26 | GGACTTCGTCT | 27 | 53.0 | 494 |
| | CCAACAGC | | CTGTCGTCTT | | | (cDNA) |
| | | | | | | 600 |
| | | | | | | (gDNA) |
| PPO8 | GATAGAAGACG | 28 | GAAGCCGATC | 29 | 54.2 | 465 |
| | CCATGCCCA | | TGGTCGTAGG | | | (cDNA) |
| | | | | | | 465 |
| | | | | | | (gDNA) |
| PPO9 | TGCTTGTTCTCG | 30 | GAAGAGGGCA | 31 | 53.4 | 455 |
| | TGGGCAT | | GAGGAGTTCA | | | (cDNA) |
| | | | | | | 1872 |
| | | | | | | (gDNA) |

The expression profile is presented in FIG. 4, depicting the average expression level of each PPO in each examined tissue. The units are arbitrary, derived by visually quantifying, expressing the quantifications as numbers, and converting the numbers to a scale of − to +, ++, +++ in a linear manner.

An additional expression analysis in some of the tissues has been performed by comparing total RNA sequencing data from various tissues and measuring expression by TMM normalization (Trimmed Mean of M-values). Briefly, samples of roots, top leaves, rolled leaves and banana flesh and peel (both green-yellow and yellow ripening stages) were harvested from greenhouse Grande Naine banana plants and commercial banana fruit, respectively. The roots, top leaves, and rolled leaves were harvested from plants grown under greenhouse conditions for 9 months to a year and a half. Samples were snap frozen and subjected to freeze drying for two days before sampling processing. Freeze dried samples were ground using a mortar and a pestle. The RNA-seq sample preparations from these samples consisted of total RNA extraction, mRNA enrichment using polyadenylation tail binding, reverse transcription to produce cDNA, and preparation of a sequencing library using adaptor ligations. The library was sequenced to a depth of at least 44 million raw reads per sample, and after raw data QC analysis (including adaptor trimming) sequencing reads were aligned to the banana genome. The number of reads aligned to each gene was quantified and normalised across samples using the ‘Trimmed Mean of M-values’ (TMM) method (Robinson, M. D., Oshlack, A. A scaling normalization method for differential expression analysis of RNA-seq data. Genome Biol 11, R25 (2010)). As can be seen in FIG. 5, high expression was seen for PPO1, PPO2, PPO8, and PPO9 at the green-yellow and/or yellow ripening stages in the flesh and/or peel. Similar expression for PPO1, PPO2, PPO8, and PPO9 was suggested by the semi-quantitative PCR data.

The annotation of the identified PPO genes was further validated on the banana genome hub (https://banana-genome-hub.southgreen.fr/) by loading additional annotation tracks onto the genome to show expression patterns from published datasets. These were assessed to confirm the expression of the annotated exon mRNA sequences in the genome. This validation confirmed, for example, the annotation of PPO1 and PPO2. The validation further confirmed that PPO3 is not expressed in any of the tissues from which RNA sequencing data was compared to the annotation. PPO1 and PPO2 were observed to be expressed in fruit flesh and peel, suggesting that they are relevant for browning of fruit, but are not expressed in embryos/ECS.

Additional RNA-seq expression analyses were performed on Grande Naine bananas obtained directly from a commercial distributor, over the course of the natural ripening process without the application of exogenous ethylene. Peel and flesh samples were harvested from five ripening stages: all-green (mature unripe stage), green-yellow (first turning point), all-yellow (mature ripe stage), yellow-brown (second turning point), all-brown (over-ripe stage). High-quality RNA was obtained from all flesh samples and from the peel of the all-green stage. Tissue samples were also harvested from leaves and roots of in vitro Grande Naine plants and from in vitro cultures of embryos and embryogenic cells. Relative mRNA abundance was quantified as described above, using TMM normalization. As can be seen in FIG. 8, PPO1, PPO4 and PPO9 account for more than 90% of PPO expression in the peel of Grande Naine bananas at the unripe green stage, whereas PPO1 is the predominant PPO gene expressed in the flesh of Grande Nanine bananas, with the exception of the over-ripe brown stage, where PPO8 is more highly expressed.

### Example 4—Confirmation of PPO Activity

To confirm that identified PPOs, such as PPO1 and PPO2, are indeed PPO genes, the proteins are synthesised and a colorimetric assay is used to test PPO activity. Briefly, for each synthesized PPO, 0.2 mL of synthesized enzyme solution and 2.8 ml of 10 mM 4-methylcatechol (0.2 M phosphate buffer, pH 6.3) are mixed, and PPO activity is measured as a function of change in absorbance at 420 nm over time (increase in absorbance indicative of PPO activity). The relative enzyme activities are calculated using the linear part of the data plot.

### Example 5—Generating a Genetically Modified Plant Edited in PPO1, PPO2, PPO8, or PPO9

In order to measure the effect of reducing the level or activity of PPO1, PPO2, PPO8, or PPO9, genetically modified banana plants are generated which are edited in either PPO1, PPO2, PPO8, or PPO9. Plants edited in PPO3 are used as controls. Agrobacterium is used to introduce DNA encoding CAS9 and an sgRNA targeting PPO1, PPO2, PPO3, PPO8, or PPO9 into the genome of the banana plant. Embryogenic cell suspensions (ECS) are transformed with an Agrobacterium strain harbouring plasmids encoding the Cas9 machinery and expressing sgRNAs targeting PPO as well as a kanamycin resistance gene (nptll). Agrobacterium transformation is performed according to Ganapathi et al., Plant Cell Reports (2001) 20:157-162, and Kanna et al., Molecular Breeding October 2004, Volume 14, Issue 3, pp 239-252. Embryogenic cells are co-cultivated with Agrobacterium for one to three days, and then transferred to regeneration medium containing G418 as selection agent until shoots develop.

CRISPR vector constructs that target each PPO gene separately are used, each with a pair of sgRNAs as shown in Tables 3 and 3A below. Tables 3 and 3A show the variable sequence of the sgRNAs, which are each used with the scaffold listed as SEQ ID NO: 98. Constructs were produced which contain combinations of sgRNA1 and sgRNA2 as shown in Table 3, to maximise the chance of generating a double mutant plant. An example of generating an edit in PPO2 is demonstrated in FIG. 6. As control, genetically modified plants targeting PPO3 are generated. The following combinations of sgRNA variable regions were used: SEQ ID NOs: 32 and 33, SEQ ID NOs: 62 and 33, SEQ ID NOs: 34 and 35, SEQ ID NOs: 36 and 37, SEQ ID NOs: 38 and 39, SEQ ID NOs: 32 and 34, SEQ ID NOs: 32 and 35, SEQ ID NOs: 33 and 34, SEQ ID NOs: 33 and 35; SEQ ID NOS: 38 and 39; and SEQ ID NOs: 57 and 58.

TABLE 3
Sequences of variable regions sgRNAs used to generate banana plants
edited in PPO genes

| | | SEQ | | SEQ |
|---|---|---|---|---|
| | | ID | | ID |
| Gene | sgRNA1 | NO: | sgRNA2 | NO: |
| PPO1 | AGGGACGTTAGTGCAGCGG | 32 | CCTCAAGGGCGAGGACGGGT | 33 |
| PPO1 | GAGGGGTTGGTTGTGGTCTC | 62 | CCTCAAGGGCGAGGACGGGT | 33 |
| PPO2 | AGGAAGCGGAGATGGTGGCA | 34 | ATGCGATGTTGGGACGAACA | 35 |
| PPO3 | GGTTAGGCTTGTCGCCCCCG | 36 | AGGACTGCTTCAAGACCCGG | 37 |
| PPO9 | AACCGATAGGTTTGCTTTGA | 38 | GGTTTGGTCGGCGTCTTTCC | 39 |
| PPO8 | ATCAGGGTAAGAAAAATGGA | 57 | CACATCGCGGCGGTCGAGCT | 58 |

TABLE 3A
Alternative sequences of variable regions sgRNAs
used to generate banana plants edited in PPO
genes

| Gene | sgRNA1 | SEQ ID NO: |
|---|---|---|
| PPO1 | CAAGCTACGCTACGAGTACC | 59 |
| PPO1 | CGCGAACTCGGATCTTGACG | 60 |
| PPO1 | GCCAAAGGCGGCGAGGACCA | 61 |
| PPO1 | GAGGGGTTGGTTGTGGTCTC | 62 |
| PPO1 | CGGAGGGAAGGGGCATGAA | 63 |
| PPO2 | AGGACTGCTTGGAGACCGAT | 64 |
| PPO2 | GTACGTGTAGCGCAGCCAAT | 65 |
| PPO2 | GGTCACGGTCAAGGACTGCT | 66 |
| PPO2 | AGAGCCAGCTGTTGTGGACTG | 67 |
| PPO2 | TTCCAGAAAGGGAGCGAGAG | 68 |
| PPO2 | TGAACAGCCTAACCCTGGCGG | 69 |
| PPO2 | AACAGCCTAACCCTGGCGCG | 70 |
| PPO3 | GGTACTTGTAGCGCAGCCAC | 71 |
| PPO3 | GTACTTGTAGCGCAGCCACC | 72 |
| PPO3 | GTTAGGCTTGTCGCCCCCG | 73 |
| PPO3 | AGGACTGCTTCAAGACCCGG | 74 |
| PPO3 | ACGGCTGAGCTTCTTCCACA | 75 |
| PPO4 | ATGGAAGTCATCCACAATG | 76 |
| PPO4 | CGTTTCGGGTCGTACAGCG | 77 |
| PPO5 | TTGCAATGTCGACCGCATGT | 78 |
| PPO5 | CGTTTCGGGTCGTACAGCG | 79 |
| PPO6 | GGCCGGTCGCCGCACCCGG | 80 |
| PPO6 | ACGGCGTGACATGCCACCAG | 81 |
| PPO7 | AATCCCCGTCACTACTGGAT | 82 |
| PPO7 | GTCCAAGTGCCACGATGCCA | 83 |
| PPO7 | CGTTGTCTTCGAACTCAATG | 84 |
| PPO7 | TCGAGCAAGTTCCTCTCCCA | 85 |
| PPO7 | CGTTGTCTTCGAACTCAATG | 86 |
| PPO7 | TGCGGTTACCGCGGAGGTTC | 87 |
| PPO7 | ACTACCTCCACTTCTACGAG | 88 |
| PPO7 | CTGGAAGTCATCCACAATG | 89 |
| PPO8 | GTCACCCCTCCGTGTCCGCC | 90 |
| PPO8 | CGCCTGGATGGGATTTCCGA | 91 |
| PPO8 | TAATCGTAACGAAGCCACTC | 92 |
| PPO8 | CCGGCTGCAAGCAGTCCTTG | 93 |
| PPO9 | ACGAGAATGTGCGCCTGCGC | 94 |
| PPO9 | ACATAGACAAACTCAGGTAC | 95 |
| PPO9 | TGCCCACGAGAACAAGCACG | 96 |
| PPO9 | GGATCTCCCGAGAGAAGCTC | 97 |

Once the genetically modified plants are regenerated, they are grown in the field until they give fruit. The assessment of PPO editing phenotype in mature plants/fruits is performed during a field trial by comparing PPO expression and browning of fruit between edited and wild type plants. About 20 plants from each edited and control line are arranged in four groups of five plants, spaced and positioned randomly in the field. Plants take about 12 weeks to flower, and the development of fruits will be assessed between about 14 to 17 weeks post flowering. For the fruits, three groups are harvested after 24 weeks from planting, and each bunch is divided and is either stored with no ripening (representative of yellow life) or ripened with ethylene, in order to test browning/PPO expression with and without induced ripening with ethylene.

Browning can be measured as described in Example 1 above.

Control plants edited in a PPO gene that is expressed in leaves (for example edited in PPO9) are compared to wild-type plants for PPO expression level and/or activity at an earlier developmental stage (as expression can be tested already in the leaves). Once it has been determined which of the genetically modified plants displays reduced browning and/or reduced PPO expression in the fruit, non-genetically modified, transiently edited banana plants edited in the selected PPO gene (not incorporating the CRISPR/CAS9 machinery into the genome) are produced.

### Example 6—Generating Banana Plants Having Mutations in PPO Genes Using Transient CAS9 Expression

In order to generate banana plants mutated in PPO genes, banana embryogenic cell suspensions (ECS) are introduced with vectors encoding the CAS9 machinery, and further expressing sgRNAs targeting a PPO gene. To produce the ECS, an embryogenic callus is first developed from an initial explant, such as immature male flowers or shoot tip as described by Ma, Proceedings of Symposium on Tissue culture of horticultural crops, Taipei, Taiwan, 8-9 Mar. 1988, pp. 181-188, and in Schoofs, H. (1997)—“The origin of embryogenic cells in Musa”, PhD thesis, KULeuven, Belgium. Embryogenic cell suspensions (ECS) are then initiated from freshly developed highly embryogenic calli in liquid medium. Next, 80% of the medium is refreshed every 12 to 14 days until the initiated cell suspension is fully established (6 to 9 months).

The embryogenic cell suspension is then bombarded with plasmids encoding the CAS9 machinery, and further expressing sgRNAs targeting PPO. Cell bombardment can be performed according to any method known in the art, such as the method described by Hamada et al., Sci Rep. 2018; 8: 14422. All the plasmids used for bombardment contain four transcriptional units. The first transcriptional unit contains the CaMV-35S promoter-driving expression of Streptococcus Cas9 (human codon optimized) and the tobacco mosaic virus (TMV) terminator. The next transcriptional unit consists of another CaMV-35S promoter driving expression of the mCherry fluorescent marker and the tNOS (nopaline synthase) terminator. The third and fourth transcriptional units each contain the wheat U6 promoter expressing sgRNA to the selected target genes (each vector comprises two sgRNAs). The sgRNAs included in the plasmids used for bombardment are designed to target PPO genes. The sgRNAs were designed to target, for example:
A region found in exon 1 of PPO gene PPO1 Ma06_g31080 (SEQ ID NO: 5);
A region found in exon 1 of PPO gene PPO2 Ma07_g03540 (SEQ ID NO: 6);
A region found in exon 2 of PPO gene PPO3 Ma07_g03650 (SEQ ID NO: 7);
A region found in exon 1 of PPO gene PPO8 Ma08_34740 (SEQ ID NO: 12); and
A region found in exon 2 of PPO gene PPO9 Ma10_g20510 (SEQ ID NO: 13).

A summary of the sgRNAs used, and the target gene used for designing them, is provided in Table 4 (all the sgRNA sequences are listed in the 5′ to 3′ direction. All sgRNAs were cloned without the sequence of the PAM motif (marked in bold typeface in Table 4).

TABLE 4
sgRNAs used to target the PPO genes

| Target gene used | | SEQ | sgRNA sequence + |
|---|---|---|---|
| for designing | sgRNA | ID | sequence of PAM |
| sgRNA | ID | NO: | motif (Bold) |
| PPO1 - Ma06_g31080 | 2019 | 49 | AGGGACGTTAGTGCAGCGGAGG |
| PPO1 - Ma06_g31080 | sg857 | 176 | GAGGGGTTGGTTGTGGTCTCTGG |
| PPO1 - Ma06_g31080 | sg858 | 50 | CCTCAAGGGCGAGGACGGGTCGG |
| PPO2 - Ma07_g03540 | sg854 | 51 | AGGAAGCGGAGATGGTGGCAGGG |
| PPO2 - Ma07_g03540 | sg855 | 52 | ATGCGATGTTGGGACGAACATGG |
| PPO3 - Ma07_g03650 | sg1435 | 53 | GGTTAGGCTTGTCGCCCCCGCGG |
| PPO3 - Ma07_g03650 | sg1436 | 54 | AGGACTGCTTCAAGACCCGGTGG |
| PPO9 - Ma10_20510 | sg850 | 55 | AACCGATAGGTTTGCTTTGAGGG |
| PPO9 - Ma10_20510 | sg851 | 56 | GGTTTGGTCGGCGTCTTTCCAGG |
| PPO8 - Ma08_34740 | sg852 | 99 | ATCAGGGTAAGAAAAATGGAAGG |
| PPO8 - Ma08_34740 | sg853 | 100 | CACATCGCGGCGGTCGAGCTTGG |

TABLE 4A
Alternative sgRNAs used to target the PPO genes

| | SEQ | |
|---|---|---|
| Target gene used for | ID | sgRNA sequence + |
| designing sgRNA | NO: | sequence of PAM motif |
| PPO1 - Ma06_g31080 | 101 | CAAGCTACGCTACGAGTACCAGG |
| PPO1 - Ma06_g31080 | 102 | CGCGAACTCGGATCTTGACGAGG |
| PPO1 - Ma06_g31080 | 103 | GCCAAAGGCGGCGAGGACCAAGG |
| PPO2 - Ma07_g03540 | 104 | AGGACTGCTTGGAGACCGATTGG |
| PPO2 - Ma07_g03540 | 105 | GTACGTGTAGCGCAGCCAATCGG |
| PPO2 - Ma07_g03540 | 106 | GGTCACGGTCAAGGACTGCTTGG |
| PPO3 - Ma07_g03650 | 107 | GGTACTTGTAGCGCAGCCACCGG |
| PPO3 - Ma07_g03650 | 108 | GTACTTGTAGCGCAGCCACCGGG |
| PPO7 - Ma08_g09180 | 109 | AATCCCCGTCACTACTGGATCGG |
| PPO7 - Ma08_g09180 | 110 | GTCCAAGTGCCACGATGCCAAGG |
| PPO7 - Ma08_g09180 | 111 | CGTTGTCTTCGAACTCAATGCGG |
| PPO7 - Ma08_g09180 | 112 | TCGAGCAAGTTCCTCTCCCATGG |
| PPO7 - Ma08_g09180 | 113 | CGTTGTCTTCGAACTCAATGCGG |
| PPO7 - Ma08_g09180 | 114 | TGCGGTTACCGCGGAGGTTCCGG |
| PPO8 - Ma08_g34740 | 115 | GTCACCCCTCCGTGTCCGCCCGG |
| PPO8 - Ma08_g34740 | 116 | CGCCTGGATGGGATTTCCGAGGG |
| PPO8 - Ma08_g34740 | 117 | TAATCGTAACGAAGCCACTCCGG |
| PPO8 - Ma08_g34740 | 118 | CCGGCTGCAAGCAGTCCTTGAGG |
| PPO9 - Ma10_g20510 | 119 | ACGAGAATGTGCGCCTGCGCAGG |
| PPO9 - Ma10_g20510 | 120 | ACATAGACAAACTCAGGTACCGG |
| PPO9 - Ma10_g20510 | 121 | TGCCCACGAGAACAAGCACGAGG |
| PPO9 - Ma10_g20510 | 122 | GGATCTCCCGAGAGAAGCTCCGG |

Three days following bombardment with the vectors containing the sgRNAs shown above, the cells are moved to a proliferation medium, followed by an embryo development medium (EDM) and then a maturation medium (relevant media can be found, for example, in Strosse H., R. Domergue, B. Panis, J. V. Escalant and F. Côte, 2003, Banana and plantain embryogenic cell suspensions (A. Vézina and C. Picq, eds). INIBAP Technical Guidelines 8, The International Network for the Improvement of Banana and Plantain, Montpellier, France). The mature embryos are germinated in germination medium (Strosse H., R. Domergue, B. Panis, J. V. Escalant and F. Côte. 2003. Banana and plantain embryogenic cell suspensions (A. Vézina and C. Picq, eds). INIBAP Technical Guidelines 8. The International Network for the Improvement of Banana and Plantain, Montpellier, France) and young shoots are transferred to shoot maturation medium until approximately 1 cm in height. Shoots are transferred to rooting medium for plantlet development.

Next, a leaf sample is taken from each plantlet for genomic DNA extraction, and the DNA samples are subjected to genotyping using Next Generation Sequencing. The purpose of the genotyping is to identify whether a Cas9-driven gene editing event has occurred in any of the PPO genes. Plants are sampled individually. A small piece of leaf is cut and placed in a sample tube (˜25 mg). Extractions of genomic DNA are made from these samples using the Sbeadex kit (Biosearch Technologies) on an OktoPure system (LGC). The resultant genomic DNA is diluted to 5 ng/ul and then mixed to form pools of 12 plants. PCR amplification of target genes is performed using primers flanking the predicted editing sites, to form 1 Kbp amplicons, using 10 ng of template DNA. The PCR amplicons for each pooled sample are mixed and sent for AmpSeq sequencing. Here, libraries are constructed using a transposase mediated fragmentation, and then miseq sequencing performed to generate reads of sequencing for each amplicon. The amplicon sequences are then analysed using the “Geneious”, “pindel”, “varscan” and “freebase” softwares to identify potential indels. If an editing event is found, it is further verified by PCR reactions using the primers listed in Table 5. When an edit is found, it indicates that the target gene was cut by transiently expressed Cas9 in the ECS from which the plantlet originated. To confirm that the Cas9 was indeed transiently expressed, the absence of Cas9 and the backbone of the carrying vector is confirmed by PCR and qRT-PCR using a standard set of primers as listed in Table 6.

In an alternative method, Sanger sequencing is used for genotyping, and the genomic DNA is used to amplify target regions using primers flanking the predicted editing sites (Table 5), and 1 kbp amplicons are analysed by Sanger sequencing to identify target gene edits.

TABLE 5
Primers used to detect gene editing events in PPO
genes

| | | | SEQ | |
|---|---|---|---|---|
| | Primer | Fwd/ | ID | Primer sequence |
| Gene ID | # | Rev | NO: | 5′ - 3′ |
| PPO1-Ma06_ | G152 | Fwd | 123 | CGAGAACCACGGTATCGATCT |
| g31080 | G159 | Rev | 124 | ATGCTGAGTGAAGTTCCGGG |
| PPO2 - Ma07_ | G170 | Fwd | 125 | ACAAGAGAACTCCAGCACGTA |
| g03540 | G171 | Rev | 126 | AGGGGCCTGAATGGGGTTAG |
| PPO3 - Ma07_ | G190 | Fwd | 127 | GCCAATTCTTCTCCAACGGC |
| g03650 | G191 | Rev | 128 | CCTTGGTGAGCTTGGGAGTC |
| PPO8 - Ma08_ | G0562 | Fwd | 129 | GATAGAAGACGCCATGCCCA |
| 34740 | G0563 | Rev | 130 | GAAGCCGATCTGGTCGTAGG |
| PPO9 - Ma10_ | G210 | Fwd | 131 | CTTTGATTTGCAACGATCTCGG |
| 20510 | G211 | Rev | 132 | TCATACTTGGCCACGAACTCC |

TABLE 6
Primers used to confirm absence of Cas9 and/or
backbone

| | | SEQ | |
|---|---|---|---|
| | | ID | |
| Gene ID | Primer # | NO: | Primer sequence 5′ - 3′ |
| Cas9 | 1684 | 133 | AGTACAAGGTGCCGAGCAAA |
| | 1685 | 134 | ACCTGAATGCAGTGGTAGGC |
| | 1686 | 135 | CCGTGCTGTTCTTTTGAGCC |
| | 1687 | 136 | GGTGGCCTTGCCTATTTCCT |
| Backbone | 1563 | 137 | ACACACGAAGCAGCAGATCA |
| detection | 1564 | 138 | ACAGCTTGCGGTACTTCTCC |
| | 1565 | 139 | AACCCAGACAACAGCGATGT |
| | 1566 | 140 | CCGTCAATGTATCCGGCGTA |
| | 1567 | 141 | AGACACGCCAGATCACCAAG |
| | 1568 | 142 | AGAAGCCTCCGGTCTGTACT |
| | 1569 | 143 | ACAAGCCTGGGGATAAGTGC |
| | 1570 | 144 | CGTTCGGTCAAGGTTCTGGA |
| | 1571 | 145 | CATCCAGAAATTGCGTGGCG |
| | 1572 | 146 | ATGACCCGACAAACAAGTGC |
| | 1573 | 147 | CTCGTGACCACCCTGACCTA |
| | 1574 | 148 | GTCCATGCCGAGAGTGATCC |
| | 1575 | 149 | GGCGGACAAGTGGTATGACA |
| | 1576 | 150 | GGCGGTGCTACAGAGTTCTT |

Once an edited plantlet is identified, the plantlet is micro-propagated to generate additional identical plantlets. Micro-propagation methods are known in the art, for example in Munir Iqbal et al. (2013), International Journal of Agriculture Innovations and Research Volume 2, Issue 1, ISSN (Online) 2319-1473. The level of PPO and browning in edited plants is then confirmed in the field as described above.

Browning can be measured as described in Example 1 above.

### Example 7-Generating Banana Plants Having Mutations in PPO Genes Using Transient Cas9 Expression in Embryogenic Cells Transformed Using Agrobacterium tumefaciens

An alternative method to that described in Example 6 for generating banana plants mutated in PPO genes utilises Agrobacterium-mediated transformation of embryogenic banana cells. Embryogenic cell suspensions (ECS) are produced as described in Example 6, and transformed using Agrobacterium tumefaciens, according to procedures such as those described by Khanna et al., Mol. Breed. 2004; 14: 239 and Tripathi et al., In Vitro Cell Dev. Biol.-Plant 2012; 48: 216. All the plasmids used for Agrobacterium-mediated transformation contain four transcriptional units. The first transcriptional unit drives expression of a resistance gene conferring resistance to a selection agent. The next transcriptional unit drives expression of human codon-optimized Streptococcus pyogenes Cas9. The third and fourth transcriptional units each drive expression of an sgRNA to the selected target genes (each vector comprises two sgRNAs). The sgRNAs included in the plasmids used for Agrobacterium-mediated transformation are designed to target PPO genes. A summary of the sgRNAs used, and the target genes used for designing them, is provided in Table 4 (all sgRNA sequences are listed in the 5′ to 3′ orientation, and sgRNAs were cloned without the sequence of the PAM motif, which is marked in bold typeface in Table 4).

After co-cultivation of banana embryogenic cells with Agrobacterium tumefaciens cells harbouring the plasmids described above, the banana cells are resuspended in 250 ml Erlenmeyer flasks in liquid proliferation medium containing the selection agent and cultured for 5 days with gentle shaking. This selection treatment allows for the enrichment of banana cells that have been successfully transformed and thereby express the resistance gene, whereas non-transformed banana cells and Agrobacterium tumefaciens cells are selected against. The banana cells are subsequently washed four times in liquid proliferation medium to remove the selection agent, and are then cultured on proliferation medium, followed by embryo development medium, maturation medium, and germination medium (relevant media can be found, for example, in Strosse H., R. Domergue, B. Panis, J. V. Escalant and F. Côte, 2003, Banana and plantain embryogenic cell suspensions (A. Vézina and C. Picq, eds). INIBAP Technical Guidelines 8, The International Network for the Improvement of Banana and Plantain, Montpellier, France). Young shoots are transferred to shoot maturation medium until approximately 1 cm in height, after which the shoots are transferred to rooting medium for plantlet development. As described in Example 6, target gene edits in regenerated plants are identified by extracting genomic DNA from leaf samples and analysing target sites by PCR and sequencing, with gene-specific primers listed in Table 5. The absence of plasmid sequences in edited plant lines is confirmed using qRT-PCR with primers listed in Table 6. Lastly, as discussed in Example 6, edited plants are micro-propagated to produce clones, and levels of enzymatic browning in the plants are validated using methods described in Example 1. The above procedure was successfully used to obtain banana plants that contain a targeted edit in the PPO1 gene and that lack extraneous plasmid sequences integrated into the genome. Banana embryogenic cells were transformed with plasmid pMOL_0019, containing sgRNAs sg857 (SEQ ID NO: 176) and sg858 (SEQ ID NO: 50) (see Table 4), both of which target the first exon of PPO1. The pMOL_0019-transformed cells were transiently selected and regenerated into shoots that were screened by Sanger sequencing and qRT-PCR as described above.

The PPO1-edited plants contain a single base pair deletion in the first exon of one of three alleles of PPO1, leading to a truncated PPO1 protein being produced from the edited allele. The resulting protein, coding, and gene sequences are provided in SEQ ID NOs: 177, 178, and 179, respectively. The target region of the PPO1 gene is depicted in FIG. 7A, and the deleted nucleotide (cytosine-bp371), sgRNAs, and genotyping primers are highlighted. The sequence of the edited and non-edited PPO1 proteins are given in FIG. 7B. As shown in Table 7, plasmid-specific primers failed to amplify target sequences from genomic DNA extracted from PPO1-edited banana plants. This was also the case for DNA from negative control wild-type plants, whereas these primers did amplify plasmid sequences from genomic DNA extracted from positive control transgenic plants. As an internal control, an endogenous banana genomic region was amplified in all samples. These analyses, therefore, confirm that plasmid sequences are absent from the genome of PPO1-edited banana plants.

TABLE 7
Cq values from quantitative PCR analyses

| | qPCR amplicons¹ | | | | | | | | |
|---|---|---|---|---|---|---|---|---|---|
| | | | | | | | | | Endogenous |
| | | | | | | | | | genomic |
| Sample | 1 | 2 | 3 | 4 | 6 | 7 | 8 | 9 | control |
| Reduced browning | NA² | NA | NA | NA | NA | NA | NA | NA | 27.2 |
| banana plant | | | | | | | | | |
| Negative control | NA | NA | NA | NA | NA | NA | NA | NA | 28.19 |
| wild-type | | | | | | | | | |
| banana plant | | | | | | | | | |
| Positive control | 23.1 | 21.46 | 21.39 | 20.84 | 23.56 | 22.86 | 22.28 | 22.48 | 30.19 |
| transgenic | | | | | | | | | |
| banana plant | | | | | | | | | |

¹Amplicon IDs from FIG. 3
²NA = Not Amplified, DNA region not present in the sample

### Example 8—Design of Further sgRNAs

Further PPO sgRNAs were designed to maximise targeting specificity (minimal number of potential off-target edits), efficiency of editing (taking into consideration potential RNA secondary structures and SNPs that affect targeting) and predictability of mutations (likelihood of inducing frameshift mutations based on the sequence microhomology surrounding the DSB). As listed in Table 8, multiple sgRNAs were designed for all PPO genes, for both Cas9 and Base editor targeting strategies, to produce either indels or programmable base substitutions, respectively, which lead to the generation of premature stop codons in the coding sequence of the target genes. The sgRNAs designed for Cas9 editing are expected to be most effective for editing using Cas9, but may also be useful for editing with base editors. Similarly, the sgRNAs designed for base editing are expected to be most effective for editing using base editors, but may also be useful for editing with Cas9. The sgRNAs against PPO3 and PPO7 were designed primarily for use as controls. The sequences in Table 8 do not include PAM sites. PAM sites can be identified by aligning guide sequences with target sequences.

TABLE 8
List of sgRNAs designed to target the PPO genes

| | | | | Target | Editing |
|---|---|---|---|---|---|
| Gene | Gene ID | sgRNA ID | sgRNA sequence | region | strategy |
| PPO1 | Ma06_g31080 | 857 | GAGGGGTTGGTTGTGGTCTC | Exon 1 | Cas9 |
| | | | | (beginning) | |
| PPO1 | Ma06_g31080 | 858 | CCTCAAGGGCGAGGACGGGT | Exon 1 | Cas9 |
| | | | | (middle) | |
| PPO1 | Ma06_g31080 | 2019 | AGGGACGTTAGTGCAGCGG | Exon 1 | Cas9 |
| | | | | (beginning) | |
| PPO1 | Ma06_g31080 | 3518 | CAAGCTACGCTACGAGTACC | Exon 2 | Cas9 |
| | | | | (middle) | |
| PPO1 | Ma06_g31080 | 3519 | GCCAAAGGCGGCGAGGACCA | Exon 2 | Cas9 |
| | | | | (end) | |
| PPO1 | Ma06_g31080 | 4117 | GCGATTGCCAGCGATGTACG | Exon 1 | Cas9 |
| | | | | (end) | |
| PPO1 | Ma06_g31080 | MolMoClo0157 | GTACAGCCAAGTCGGCTTCC | Exon 1 | Base |
| | | | | (middle) | editor |
| PPO1 | Ma06_g31080 | MolMoClo0158 | AGAGCCATGAGTTGTGCACC | Exon 1 | Base |
| | | | | (middle) | editor |
| PPO1 | Ma06_g31080 | MolMoClo0159 | TCGCCGGTCCAGACGTGGAC | Exon 2 | Base |
| | | | | (beginning) | editor |
| PPO1 | Ma06_g31080 | MolMoClo0160 | CGAGCCAGTCGGGGTCGGCC | Exon 2 | Base |
| | | | | (middle) | editor |
| PPO2 | Ma07_g03540 | 854 | AGGAAGCGGAGATGGTGGCA | Exon 1 | Cas9 |
| | | | | (beginning) | |
| PPO2 | Ma07_g03540 | 855 | ATGCGATGTTGGGACGAACA | Exon 1 | Cas9 |
| | | | | (beginning) | |
| PPO2 | Ma07_g03540 | 3520 | GGTCACGGTCAAGGACTGCT | Exon 1 | Cas9 |
| | | | | (end) | |
| PPO2 | Ma07_g03540 | 2961 | AGAGCCAGCTGTTGTGGACT | Exon 1 | Base |
| | | | | (middle) | editor |
| PPO2 | Ma07_g03540 | 2962 | GTTCCAGAAAGGGAGCGAGA | Exon 1 | Base |
| | | | | (middle) | editor |
| PPO2 | Ma07_g03540 | 2963 | TGAACAGCCTAACCCTGGCG | Exon 1 | Base |
| | | | | (middle) | editor |
| PPO2 | Ma07_g03540 | 2964 | GAACAGCCTAACCCTGGCGC | Exon 1 | Base |
| | | | | (middle) | editor |
| PPO3 | Ma07_g03650 | 1435 | GGTTAGGCTTGTCGCCCCCG | Exon 2 | Cas9 |
| | | | | (middle) | |
| PPO3 | Ma07_g03650 | 1436 | AGGACTGCTTCAAGACCCGG | Exon 2 | Cas9 |
| | | | | (middle) | |
| PPO4 | Ma08_g09150 | 4118 | ATGTCGCGCCGATCCAGCAG | Exon 1 | Cas9 |
| | | | | (beginning) | |
| PPO4, | Ma08_g09150, | 4119 | TGCGGTCACCATTACTGCCC | Exon 1 | Cas9 |
| PPO5 | Ma08_g09160 | | | (beginning) | |
| PPO6 | Ma08_g09170 | 4120 | TCGGCGCGACATGCTGTTGG | Exon 1 | Cas9 |
| | | | | (beginning) | |
| PPO6 | Ma08_g09170 | 4121 | GGCTTTACGGCGTGACCGCA | Exon 1 | Cas9 |
| | | | | (beginning) | |
| PPO7 | Ma08_g09180 | 3521 | AATCCCCGTCACTACTGGAT | Exon 1 | Cas9 |
| | | | | (beginning) | |
| PPO7 | Ma08_g09180 | 3522 | GTCCAAGTGCCACGATGCCA | Exon 1 | Cas9 |
| | | | | (beginning) | |
| PPO7 | Ma08_g09180 | 3523 | TCGAGCAAGTTCCTCTCCCA | Exon 2 | Cas9 |
| | | | | (beginning) | |
| PPO7 | Ma08_g09180 | 3524 | TGCGGTTACCGCGGAGGTTC | Exon 1 | Cas9 |
| | | | | (end) | |
| PPO8 | Ma08_g34740 | 3525 | GTCACCCCTCCGTGTCCGCC | Exon 1 | Cas9 |
| | | | | (beginning) | |
| PPO8 | Ma08_g34740 | 3526 | CGCCTGGATGGGATTTCCGA | Exon 1 | Cas9 |
| | | | | (beginning) | |
| PPO8 | Ma08_g34740 | 3527 | TAATCGTAACGAAGCCACTC | Exon 1 | Cas9 |
| | | | | (end) | |
| PPO8 | Ma08_g34740 | 4158 | GGTAAGATCAGGCGCCTGGA | Exon 1 | Cas9 |
| | | | | (beginning) | |
| PPO8 | Ma08_g34740 | 4159 | CATGAAGTTGCGCGGATCGT | Exon 1 | Cas9 |
| | | | | (beginning) | |
| PPO8 | Ma08_g34740 | MolMoClo0161 | AGCTCCAAGTCCACAACTCC | Exon 1 | Base |
| | | | | (middle) | editor |
| PPO8 | Ma08_g34740 | MolMoClo0162 | CGTCCCAGTTCCAGAAAGGA | Exon 1 | Base |
| | | | | (middle) | editor |
| PPO8 | Ma08_g34740 | MolMoClo0163 | TACCGGCAGGTGATCTCCAA | Exon 1 | Base |
| | | | | (middle) | editor |
| PPO8 | Ma08_g34740 | MolMoClo0164 | GTCGCCAGTCCACCCGTGGA | Exon 1 | Base |
| | | | | (middle) | editor |
| PPO9 | Ma10_g20510 | 850 | AACCGATAGGTTTGCTTTGA | Exon 2 | Cas9 |
| | | | | (beginning) | |
| PPO9 | Ma10_g20510 | 851 | GGTTTGGTCGGCGTCTTTCC | Exon 2 | Cas9 |
| | | | | (beginning) | |
| PPO9 | Ma10_g20510 | 3528 | ACGAGAATGTGCGCCTGCGC | Exon 2 | Cas9 |
| | | | | (middle) | |
| PPO9 | Ma10_g20510 | 3529 | TGCCCACGAGAACAAGCACG | Exon 1 | Cas9 |
| | | | | (beginning) | |
| PPO9 | Ma10_g20510 | 4160 | CAGGCCGGGCAACAGTAGAC | Exon 2 | Cas9 |
| | | | | (beginning) | |
| PPO9 | Ma10_g20510 | MolMoClo0165 | GGCCTGGCGCCAAAAGTTGT | Exon 2 | Base |
| | | | | (beginning) | editor |
| PPO9 | Ma10_g20510 | MolMoClo0166 | GGGGGTGTCCCAGCTCCAGT | Exon 2 | Base |
| | | | | (middle) | editor |
| PPO9 | Ma10_g20510 | MolMoClo0167 | TCGGGGAACCACATGCCCTC | Exon 2 | Base |
| | | | | (middle) | editor |
| PPO9 | Ma10_g20510 | MolMoClo0168 | ATCACACCACCCCTCGACGC | Exon 2 | Base |
| | | | | (middle) | editor |
| PPO9 | Ma10_g20510 | MolMoClo0169 | CGACCAGGAGTGGCTCGAGT | Exon 2 | Base |
| | | | | (middle) | editor |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned herein are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of reducing the level or activity of at least one endogenous PPO1, PPO2, PPO8, PPO9, or PPO4 polyphenol oxidase encoded by a PPO1, PPO2, PPO8, PPO9, or PPO4 polyphenol oxidase gene in a banana plant or banana plant cell.

2. The method of claim 1, wherein the PPO1, PPO2, PPO8, PPO9, or PPO4 polyphenol oxidase gene:
(A) comprises a coding sequence selected from the group consisting of:
(a) SEQ ID NO: 5 (PPO1) or a polynucleotide with at least 75% sequence identity to SEQ ID NO: 5;
(b) SEQ ID NO: 6 (PPO2) or a polynucleotide with at least 75% sequence identity to SEQ ID NO: 6;
(c) SEQ ID NO: 12 (PPO8) or a polynucleotide with at least 75% sequence identity to SEQ ID NO: 12;
(d) SEQ ID NO: 13 (PPO9) or a polynucleotide with at least 75% sequence identity to SEQ ID NO: 13; and
(e) SEQ ID NO: 8 (PPO4) or a polynucleotide with at least 75% sequence identity to SEQ ID NO: 8;
(B) encodes a polyphenol oxidase selected from the group consisting of:
(a) SEQ ID NO: 40 (PPO1) or a polypeptide with at least 75% sequence identity to SEQ ID NO: 40;
(b) SEQ ID NO: 41 (PPO2) or a polypeptide with at least 75% sequence identity to SEQ ID NO: 41;
(c) SEQ ID NO: 47 (PPO8) or a polypeptide with at least 75% sequence identity to SEQ ID NO: 47;
(d) SEQ ID NO: 48 (PPO9) or a polypeptide with at least 75% sequence identity to SEQ ID NO: 48; and
(e) SEQ ID NO: 43 (PPO4) or a polypeptide with at least 75% sequence identity to SEQ ID NO: 43; or
(C) comprises a polynucleotide sequence selected from the group consisting of:
(a) SEQ ID NO: 151 (PPO1) or a polynucleotide with at least 75% sequence identity to SEQ ID NO: 151;
(b) SEQ ID NO: 152 (PPO2) or a polynucleotide with at least 75% sequence identity to SEQ ID NO: 152;
(c) SEQ ID NO: 158 (PPO8) or a polynucleotide with at least 75% sequence identity to SEQ ID NO: 158;
(d) SEQ ID NO: 159 (PPO9) or a polynucleotide with at least 75% sequence identity to SEQ ID NO: 159; and
(e) SEQ ID NO: 154 (PPO4) or a polynucleotide with at least 75% sequence identity to SEQ ID NO: 154.

3. The method of claim 1 or 2, wherein the method results in:
(a) a reduction in level of the at least one endogenous PPO1, PPO2, PPO8, PPO9, or PPO4 polyphenol oxidase in said banana plant or banana plant cell;
(b) a reduction in function of the at least one endogenous PPO1, PPO2, PPO8, PPO9, or PPO4 polyphenol oxidase in said banana plant or banana plant cell; or
(c) a loss of function of the at least one endogenous PPO1, PPO2, PPO8, PPO9, or PPO4 polyphenol oxidase in said banana plant or banana plant cell.

4. The method of any one of claims 1 to 3, wherein the method results in:
(a) delayed browning of fruit flesh and/or fruit peel of said banana plant as compared to fruit flesh and/or fruit peel of a banana plant in which the level or activity of said at least one endogenous PPO1, PPO2, PPO8, PPO9, or PPO4 polyphenol oxidase is not reduced; and/or
(b) reduced browning of fruit flesh and/or fruit peel of said banana plant as compared to fruit flesh and/or fruit peel of a banana plant in which the level or activity of said at least one endogenous PPO1, PPO2, PPO8, PPO9, or PPO4 polyphenol oxidase is not reduced.

5. The method of any one of claims 1 to 4, wherein the method comprises:
(a) providing to said banana plant cell or to a part of said banana plant a silencing RNA targeting a transcript of said at least one PPO1, PPO2, PPO8, PPO9, or PPO4 polyphenol oxidase gene, optionally wherein providing said silencing RNA is by introducing an endonuclease to said banana plant cell or part of said banana plant, said endonuclease being capable of modifying a gene encoding an endogenous non-coding RNA such that it encodes said silencing RNA targeting a transcript of said at least one PPO1, PPO2, PPO8, PPO9, or PPO4 polyphenol oxidase gene, further optionally wherein the endonuclease is selected from the group consisting of: a meganuclease, a zinc finger nuclease (ZFN), a transcription-activator like effector nuclease (TALEN), a homing endonuclease, a CRISPR-associated endonuclease and a modified CRISPR-associated endonuclease; or
(b) introducing a modification into the PPO1, PPO2, PPO8, PPO9, or PPO4 polyphenol oxidase gene encoding said at least one PPO1, PPO2, PPO8, PPO9, or PPO4 polyphenol oxidase; optionally wherein said modification is introduced into said the PPO1, PPO2, PPO8, PPO9, or PPO4 polyphenol oxidase gene by an endonuclease provided to said banana plant cell and capable of targeting said at least one PPO1, PPO2, PPO8, PPO9, or PPO4 polyphenol oxidase gene; further optionally wherein the endonuclease is selected from the group consisting of: a meganuclease, a zinc finger nuclease (ZFN), a transcription-activator like effector nuclease (TALEN), a homing endonuclease, a CRISPR-associated endonuclease, and a modified CRISPR-associated endonuclease;
further optionally wherein said CRISPR-associated endonuclease is a Cas9 endonuclease.

6. The method of any one of claims 1 to 4, wherein the method comprises providing to said banana plant cell a CRISPR-associated endonuclease or a modified CRISPR-associated endonuclease, and one or more guide RNAs specific to said at least one PPO1, PPO2, PPO8, PPO9, or PPO4 polyphenol oxidase gene, wherein said CRISPR-associated endonuclease or modified CRISPR-associated endonuclease, and said one or more guide RNAs, form a complex that enables the CRISPR-associated endonuclease or modified CRISPR-associated endonuclease to introduce a double-strand or single-strand break at said at least one endogenous PPO1, PPO2, PPO8, PPO9, or PPO4 polyphenol oxidase gene; optionally wherein said CRISPR-associated endonuclease is a Cas9 endonuclease.

7. The method of claim 5 or 6, further comprising identifying at least one banana plant cell that comprises a modification of the at least one endogenous PPO1, PPO2, PPO8, PPO9, or PPO4 polyphenol oxidase gene, wherein the modification is selected from the group consisting of:
(a) at least one nucleotide insertion;
(b) at least one nucleotide deletion;
(c) an insertion-deletion (indel);
(d) an inversion;
(e) at least one nucleotide substitution; and
(f) any combination of (a) to (e).

8. The method of claim 6 or 7, wherein said one or more guide RNAs are provided to the banana plant cell within one or more recombinant DNA constructs encoding said one or more guide RNAs operably linked to one or more promoters.

9. The method of claim 6 or 7, wherein said CRISPR-associated endonuclease or modified CRISPR-associated endonuclease, and/or said one or more guide RNAs, are provided to said banana plant cell in RNA form.

10. The method of claim 6 or 7, wherein said CRISPR-associated endonuclease or modified CRISPR-associated endonuclease is provided to said banana plant cell in protein form and said one or more guide RNAs are provided to said banana plant cell in RNA form; optionally wherein said CRISPR-associated endonuclease or modified CRISPR-associated endonuclease and said one or more guide RNAs are provided to said banana plant cell as a ribonucleoprotein complex.

11. The method of any one of claims 5 to 10, wherein said endonuclease, said one or more guide RNAs and/or said one or more recombinant DNA constructs are provided to said banana plant cell using a method selected from the group consisting of:
(a) particle bombardment;
(b) Agrobacterium transformation;
(c) protoplast transfection;
(d) electroporation; and
(e) nanoparticle-mediated transfection.

12. The method of any one of claims 5 to 11, wherein said endonuclease is provided to the banana plant cell as a polynucleotide encoding an endonuclease polypeptide; optionally wherein said endonuclease is Cas9 endonuclease and the polynucleotide is a Cas9 polynucleotide encoding a Cas9 polypeptide.

13. The method of claim 6 or 7, wherein said one or more guide RNAs and said CRISPR-associated endonuclease or modified CRISPR-associated endonuclease are provided to said banana plant cell via Agrobacterium transformation of one or more plasmids encoding said CRISPR-associated endonuclease or modified CRISPR-associated endonuclease, at least one selectable marker gene, and said one or more guide RNAs.

14. The method of any one of claims 6 to 13, wherein said one or more guide RNAs comprises a variable region having a sequence selected from the group consisting of:
(a) SEQ ID NO: 32;
(b) SEQ ID NO: 33;
(c) SEQ ID NO: 34;
(d) SEQ ID NO: 35;
(e) SEQ ID NO: 57;
(f) SEQ ID NO: 58;
(g) SEQ ID NO: 38;
(h) SEQ ID NO: 39;
(i) SEQ ID NO: 62;
(j) SEQ ID NO: 76;
(k) SEQ ID NO: 77; and
(l) any combination of (a) to (k).

15. The method of any one of claims 6 to 13, wherein said one or more guide RNAs are a pair of guide RNAs comprising variable regions selected from the group consisting of:
(a) SEQ ID NOs: 32 and 33;
(b) SEQ ID NOs: 34 and 35;
(c) SEQ ID NOs: 32 and 34;
(d) SEQ ID NOs: 32 and 35;
(e) SEQ ID NOs: 33 and 34;
(f) SEQ ID NOs: 33 and 35;
(g) SEQ ID NOs: 57 and 58;
(h) SEQ ID NOs: 38 and 39;
(i) SEQ ID NOs: 62 and 33; and
(j) SEQ ID NOs: 76 and 77.

16. The method of any one of claims 1 to 15, wherein said banana plant cell is an embryogenic cell and/or contained in an embryogenic cell suspension.

17. A banana plant cell obtainable by the method of any one of claims 1 to 16.

18. The method of any one of claims 1 to 16, further comprising regenerating a banana plant from said banana plant cell;
optionally further comprising harvesting fruit from said banana plant.

19. A banana plant or plant part obtainable by the method of claim 18; optionally wherein the banana plant or plant part comprises a mutated PPO1 gene:
(a) as set forth in SEQ ID NO: 179;
(b) that expresses a truncated PPO1 protein as set forth in SEQ ID NO: 177; or
(c) that has a coding sequence as set forth in SEQ ID NO: 178;
further optionally wherein the mutation is present in only one allele of the PPO1 gene.

20. Fruit harvested from a banana plant obtainable by the method of claim 18, wherein said fruit flesh and/or fruit peel is characterised by a phenotype of delayed and/or reduced browning as compared to fruit flesh and/or fruit peel of a banana plant in which the level or activity of said at least one endogenous PPO1, PPO2, PPO8, PPO9, or PPO4 polyphenol oxidase is not reduced.

21. A method of producing a banana plant characterised by a phenotype of delayed and/or reduced browning of fruit flesh and/or peel as compared to a wild-type banana plant, the method comprising:
(a) providing to a banana plant cell a CRISPR-associated endonuclease or a modified CRISPR-associated endonuclease, and one or more guide RNAs, wherein said CRISPR-associated endonuclease or modified CRISPR-associated endonuclease and said one or more guide RNAs form a complex that enables the CRISPR-associated endonuclease or modified CRISPR-associated endonuclease to introduce a single-strand break or a double-strand break in at least one endogenous PPO1, PPO2, PPO8, PPO9, or PPO4 polyphenol oxidase gene:
(A) comprising a coding sequence selected from the group consisting of:
(i) SEQ ID NO: 5 or a polynucleotide with at least 75% sequence identity to SEQ ID NO: 5;
(ii) SEQ ID NO: 6 or a polynucleotide with at least 75% sequence identity to SEQ ID NO: 6;
(iii) SEQ ID NO: 12 or a polynucleotide with at least 75% sequence identity to SEQ ID NO: 12;
(iv) SEQ ID NO: 13 or a polynucleotide with at least 75% sequence identity to SEQ ID NO: 13; and
(v) SEQ ID NO: 8 or a polynucleotide with at least 75% sequence identity to SEQ ID NO: 8;
(B) encoding a polyphenol oxidase selected from the group consisting of:
(i) SEQ ID NO: 40 or a polypeptide with at least 75% sequence identity to SEQ ID NO: 40;
(ii) SEQ ID NO: 41 or a polypeptide with at least 75% sequence identity to SEQ ID NO: 41;
(iii) SEQ ID NO: 47 or a polypeptide with at least 75% sequence identity to SEQ ID NO: 47;
(iv) SEQ ID NO: 48 or a polypeptide with at least 75% sequence identity to SEQ ID NO: 48; and
(v) SEQ ID NO: 43 or a polypeptide with at least 75% sequence identity to SEQ ID NO: 43; or
(C) comprising a polynucleotide sequence selected from the group consisting of:
(i) SEQ ID NO: 151 or a polynucleotide with at least 75% sequence identity to SEQ ID NO: 151;
(ii) SEQ ID NO: 152 or a polynucleotide with at least 75% sequence identity to SEQ ID NO: 152;
(iii) SEQ ID NO: 158 or a polynucleotide with at least 75% sequence identity to SEQ ID NO: 158;
(iv) SEQ ID NO: 159 or a polynucleotide with at least 75% sequence identity to SEQ ID NO: 159; and
(v) SEQ ID NO: 154 or a polynucleotide with at least 75% sequence identity to SEQ ID NO: 154;
(b) identifying at least one banana plant cell that comprises a modification of said at least one endogenous PPO1, PPO2, PPO8, PPO9, or PPO4 polyphenol oxidase gene, wherein the modification is selected from the group consisting of:
(i) at least one nucleotide insertion;
(ii) at least one nucleotide deletion;
(iii) at least one nucleotide substitution; or
(iv) any combination of (b)(i) to (b)(iii); and
(c) regenerating a banana plant from said banana plant cell, wherein said banana plant is characterised by a phenotype of delayed and/or reduced browning of fruit flesh and/or peel as compared to a wild-type banana plant.

22. The method of claim 21, further comprising harvesting fruit from said banana plant, wherein said fruit is characterised by a phenotype of delayed and/or reduced browning as compared to fruit of a banana plant in which the level or activity of said at least one endogenous PPO1, PPO2, PPO8, PPO9, or PPO4 polyphenol oxidase is not reduced or lost.

23. A banana plant or plant part comprising in its genome at least one modified endogenous PPO1, PPO2, PPO8, PPO9, or PPO4 polyphenol oxidase gene, wherein said modification results in a reduction in, or loss of function of, the at least one endogenous PPO1, PPO2, PPO8, PPO9, or PPO4 polyphenol oxidase encoded by said modified endogenous PPO1, PPO2, PPO8, PPO9, or PPO4 polyphenol oxidase gene, wherein said modification is located in at least one endogenous PPO1, PPO2, PPO8, PPO9, or PPO4 polyphenol oxidase gene:
(A) comprising a coding sequence selected from the group consisting of:
(a) SEQ ID NO: 5 or a polynucleotide with at least 75% sequence identity to SEQ ID NO: 5;
(b) SEQ ID NO: 6 or a polynucleotide with at least 75% sequence identity to SEQ ID NO: 6;
(c) SEQ ID NO: 12 or a polynucleotide with at least 75% sequence identity to SEQ ID NO: 12;
(d) SEQ ID NO: 13 or a polynucleotide with at least 75% sequence identity to SEQ ID NO: 13; and
(e) SEQ ID NO: 8 or a polynucleotide with at least 75% sequence identity to SEQ ID NO: 8;
(B) encoding a polyphenol oxidase selected from the group consisting of:
(a) SEQ ID NO: 40 or a polypeptide with at least 75% sequence identity to SEQ ID NO: 40;
(b) SEQ ID NO: 41 or a polypeptide with at least 75% sequence identity to SEQ ID NO: 41;
(c) SEQ ID NO: 47 or a polypeptide with at least 75% sequence identity to SEQ ID NO: 47;
(d) SEQ ID NO: 48 or a polypeptide with at least 75% sequence identity to SEQ ID NO: 48; and
(e) SEQ ID NO: 43 or a polypeptide with at least 75% sequence identity to SEQ ID NO: 43; or
(C) comprising a polynucleotide sequence selected from the group consisting of:
(a) SEQ ID NO: 151 or a polynucleotide with at least 75% sequence identity to SEQ ID NO: 151;
(b) SEQ ID NO: 152 or a polynucleotide with at least 75% sequence identity to SEQ ID NO: 152;
(c) SEQ ID NO: 158 or a polynucleotide with at least 75% sequence identity to SEQ ID NO: 158;
(d) SEQ ID NO: 159 or a polynucleotide with at least 75% sequence identity to SEQ ID NO: 159; and
(e) SEQ ID NO: 154 or a polynucleotide with at least 75% sequence identity to SEQ ID NO: 154.

24. The banana plant or plant part of claim 19 or 23, wherein the banana plant or plant part is non-transgenic.

25. Banana fruit harvested from the banana plant of claim 23 or 24, wherein said fruit is characterised by a phenotype of delayed and/or reduced browning as compared to fruit of a banana plant in which the level or activity of said at least one endogenous PPO1, PPO2, PPO8, PPO9, or PPO4 polyphenol oxidase is not reduced.

26. A method of obtaining a banana fruit food product, the method comprising processing the banana fruit of claim 25.

27. A DNA sequence comprising a banana PPO1, PPO2, PPO8, PPO9, or PPO4 polyphenol oxidase polynucleotide:
(A) comprising a coding sequence selected from the group consisting of:
(a) SEQ ID NO: 5 or a polynucleotide with at least 75% sequence identity to SEQ ID NO: 5;
(b) SEQ ID NO: 6 or a polynucleotide with at least 75% sequence identity to SEQ ID NO: 6;
(c) SEQ ID NO: 12 or a polynucleotide with at least 75% sequence identity to SEQ ID NO: 12;
(d) SEQ ID NO: 13 or a polynucleotide with at least 75% sequence identity to SEQ ID NO: 13; and
(e) SEQ ID NO: 8 or a polynucleotide with at least 75% sequence identity to SEQ ID NO: 8;
(B) encoding a polyphenol oxidase selected from the group consisting of:
(a) SEQ ID NO: 40 or a polypeptide with at least 75% sequence identity to SEQ ID NO: 40;
(b) SEQ ID NO: 41 or a polypeptide with at least 75% sequence identity to SEQ ID NO: 41;
(c) SEQ ID NO: 47 or a polypeptide with at least 75% sequence identity to SEQ ID NO: 47;
(d) SEQ ID NO: 48 or a polypeptide with at least 75% sequence identity to SEQ ID NO: 48; and
(e) SEQ ID NO: 43 or a polypeptide with at least 75% sequence identity to SEQ ID NO: 43; or
(C) comprising a polynucleotide sequence selected from the group consisting of:
(a) SEQ ID NO: 151 or a polynucleotide with at least 75% sequence identity to SEQ ID NO: 151;
(b) SEQ ID NO: 152 or a polynucleotide with at least 75% sequence identity to SEQ ID NO: 152;
(c) SEQ ID NO: 158 or a polynucleotide with at least 75% sequence identity to SEQ ID NO: 158;
(d) SEQ ID NO: 159 or a polynucleotide with at least 75% sequence identity to SEQ ID NO: 159; and
(e) SEQ ID NO: 154 or a polynucleotide with at least 75% sequence identity to SEQ ID NO: 154.

28. A DNA construct or vector comprising a banana PPO1, PPO2, PPO8, PPO9, or PPO4 polyphenol oxidase polynucleotide:
(A) comprising a coding sequence selected from the group consisting of:
(a) SEQ ID NO: 5 or a polynucleotide with at least 75% sequence identity to SEQ ID NO: 5;
(b) SEQ ID NO: 6 or a polynucleotide with at least 75% sequence identity to SEQ ID NO: 6;
(c) SEQ ID NO: 12 or a polynucleotide with at least 75% sequence identity to SEQ ID NO: 12;
(d) SEQ ID NO: 13 or a polynucleotide with at least 75% sequence identity to SEQ ID NO: 13; and
(e) SEQ ID NO: 8 or a polynucleotide with at least 75% sequence identity to SEQ ID NO: 8;
(B) encoding a polyphenol oxidase selected from the group consisting of:
(a) SEQ ID NO: 40 or a polypeptide with at least 75% sequence identity to SEQ ID NO: 40;
(b) SEQ ID NO: 41 or a polypeptide with at least 75% sequence identity to SEQ ID NO: 41;
(c) SEQ ID NO: 47 or a polypeptide with at least 75% sequence identity to SEQ ID NO: 47;
(d) SEQ ID NO: 48 or a polypeptide with at least 75% sequence identity to SEQ ID NO: 48; and
(e) SEQ ID NO: 43 or a polypeptide with at least 75% sequence identity to SEQ ID NO: 43; or
(C) comprising a polynucleotide sequence selected from the group consisting of:
(a) SEQ ID NO: 151 or a polynucleotide with at least 75% sequence identity to SEQ ID NO: 151;
(b) SEQ ID NO: 152 or a polynucleotide with at least 75% sequence identity to SEQ ID NO: 152;
(c) SEQ ID NO: 158 or a polynucleotide with at least 75% sequence identity to SEQ ID NO: 158;
(d) SEQ ID NO: 159 or a polynucleotide with at least 75% sequence identity to SEQ ID NO: 159; and
(e) SEQ ID NO: 154 or a polynucleotide with at least 75% sequence identity to SEQ ID NO: 154.

29. A plant cell transformed with the vector or claim 28, optionally wherein the plant cell is a banana plant cell.

30. A polyphenol oxidase protein:
(a) encoded by SEQ ID NO: 5, SEQ ID NO: 6, SEQ ID NO: 12, SEQ ID NO: 13, or SEQ ID NO: 8, or encoded by a polynucleotide with at least 75% sequence identity to SEQ ID NO: 5, SEQ ID NO: 6, SEQ ID NO: 12, SEQ ID NO: 13, or SEQ ID NO: 8;
(b) comprising SEQ ID NO: 40, SEQ ID NO: 41, SEQ ID NO: 47, SEQ ID NO: 48, or SEQ ID NO: 43, or comprising a sequence with at least 75% sequence identity to SEQ ID NO: 40, SEQ ID NO: 41, SEQ ID NO: 47, SEQ ID NO: 48, or SEQ ID NO: 43; or
(c) encoded by SEQ ID NO: 151, SEQ ID NO: 152, SEQ ID NO: 158, SEQ ID NO: 159, or SEQ ID NO: 154, or encoded by a polynucleotide with at least 75% sequence identity to SEQ ID NO: 151, SEQ ID NO: 152, SEQ ID NO: 158, SEQ ID NO: 159, or SEQ ID NO: 154.

31. A method of expressing a polyphenol oxidase in a plant cell, wherein the method comprises introducing into said plant cell a banana polyphenol oxidase polynucleotide:
(A) comprising a coding sequence selected from the group consisting of:
(a) SEQ ID NO: 5 or a polynucleotide with at least 75% sequence identity to SEQ ID NO: 5;
(b) SEQ ID NO: 6 or a polynucleotide with at least 75% sequence identity to SEQ ID NO: 6;
(c) SEQ ID NO: 12 or a polynucleotide with at least 75% sequence identity to SEQ ID NO: 12;
(d) SEQ ID NO: 13 or a polynucleotide with at least 75% sequence identity to SEQ ID NO: 13; and
(e) SEQ ID NO: 8 or a polynucleotide with at least 75% sequence identity to SEQ ID NO: 8;
(B) encoding a polyphenol oxidase selected from the group consisting of:
(a) SEQ ID NO: 40 or a polypeptide with at least 75% sequence identity to SEQ ID NO: 40;
(b) SEQ ID NO: 41 or a polypeptide with at least 75% sequence identity to SEQ ID NO: 41;
(c) SEQ ID NO: 47 or a polypeptide with at least 75% sequence identity to SEQ ID NO: 47;
(d) SEQ ID NO: 48 or a polypeptide with at least 75% sequence identity to SEQ ID NO: 48; and
(e) SEQ ID NO: 43 or a polypeptide with at least 75% sequence identity to SEQ ID NO: 43; or
(C) comprising a polynucleotide sequence selected from the group consisting of:
(a) SEQ ID NO: 151 or a polynucleotide with at least 75% sequence identity to SEQ ID NO: 151;
(b) SEQ ID NO: 152 or a polynucleotide with at least 75% sequence identity to SEQ ID NO: 152;
(c) SEQ ID NO: 158 or a polynucleotide with at least 75% sequence identity to SEQ ID NO: 158;
(d) SEQ ID NO: 159 or a polynucleotide with at least 75% sequence identity to SEQ ID NO: 159; and
(e) SEQ ID NO: 154 or a polynucleotide with at least 75% sequence identity to SEQ ID NO: 154;
operably linked to a promoter active in a plant cell.

32. A synthetic banana polyphenol oxidase guide RNA comprising a variable region selected from the group consisting of:
(a) SEQ ID NO: 32;
(b) SEQ ID NO: 33;
(c) SEQ ID NO: 34;
(d) SEQ ID NO: 35;
(e) SEQ ID NO: 57;
(f) SEQ ID NO: 58;
(g) SEQ ID NO: 38;
(h) SEQ ID NO: 39;
(i) SEQ ID NO: 62;
(j) SEQ ID NO: 76; and
(k) SEQ ID NO: 77.

33. A recombinant DNA construct comprising a promoter operably linked to a nucleotide sequence expressing at least one banana PPO1, PPO2, PPO8, PPO9, or PPO4 polyphenol oxidase guide RNA, wherein said guide RNA is capable of forming a complex with a CRISPR-associated endonuclease or modified CRISPR-associated endonuclease, and wherein said complex is capable of binding to and creating a double-strand or single-strand break in the at least one endogenous banana PPO1, PPO2, PPO8, PPO9, or PPO4 polyphenol oxidase gene:
(A) comprising a coding sequence selected from the group consisting of:
(a) SEQ ID NO: 5 or a polynucleotide with at least 75% sequence identity to SEQ ID NO: 5;
(b) SEQ ID NO: 6 or a polynucleotide with at least 75% sequence identity to SEQ ID NO: 6;
(c) SEQ ID NO: 12 or a polynucleotide with at least 75% sequence identity to SEQ ID NO: 12;
(d) SEQ ID NO: 13 or a polynucleotide with at least 75% sequence identity to SEQ ID NO: 13; and
(e) SEQ ID NO: 8 or a polynucleotide with at least 75% sequence identity to SEQ ID NO: 8;
(B) encoding a polyphenol oxidase selected from the group consisting of:
(a) SEQ ID NO: 40 or a polypeptide with at least 75% sequence identity to SEQ ID NO: 40;
(b) SEQ ID NO: 41 or a polypeptide with at least 75% sequence identity to SEQ ID NO: 41;
(c) SEQ ID NO: 47 or a polypeptide with at least 75% sequence identity to SEQ ID NO: 47;
(d) SEQ ID NO: 48 or a polypeptide with at least 75% sequence identity to SEQ ID NO: 48; and
(e) SEQ ID NO: 43 or a polypeptide with at least 75% sequence identity to SEQ ID NO: 43; or
(C) comprising a polynucleotide sequence selected from the group consisting of:
(a) SEQ ID NO: 151 or a polynucleotide with at least 75% sequence identity to SEQ ID NO: 151;
(b) SEQ ID NO: 152 or a polynucleotide with at least 75% sequence identity to SEQ ID NO: 152;
(c) SEQ ID NO: 158 or a polynucleotide with at least 75% sequence identity to SEQ ID NO: 158;
(d) SEQ ID NO: 159 or a polynucleotide with at least 75% sequence identity to SEQ ID NO: 159; and
(e) SEQ ID NO: 154 or a polynucleotide with at least 75% sequence identity to SEQ ID NO: 154;
in the banana genome.

34. The recombinant DNA construct of claim 33, wherein the one or more banana polyphenol oxidase guide RNAs comprises a variable region having a sequence selected from the group consisting of:
(a) SEQ ID NO: 32;
(b) SEQ ID NO: 33;
(c) SEQ ID NO: 34;
(d) SEQ ID NO: 35;
(e) SEQ ID NO: 57;
(f) SEQ ID NO: 58;
(g) SEQ ID NO: 38;
(h) SEQ ID NO: 39;
(i) SEQ ID NO: 62;
(j) SEQ ID NO: 76;
(k) SEQ ID NO: 77; and
(l) any combination of (a) to (k).

35. A recombinant DNA construct of claim 33 or 34, wherein said CRISPR-associated endonuclease is a Cas9 endonuclease.


SEQUENCE LISTING (225 sequences)

SEQ ID NO: 1 - protein, 586 aa, Malus domestica
MTSSPLPPTSTMAALHSTTTTTLFRSPLFPNKSQTPLQRKPKQCLAGRVRCKATKGDNDNLDQGLARLDRRNMLIGLGTGGLYSAAGNSFAFAAPVSAPDLTTCGPADKPDGSTIDCCPPITTTIIDFKLPDRGPLRTRIAAQDVAKNPAYLAKYKKAIELMRALPDDDPRSLVQQAKVHCSYCDGGYPQVGYSDLEIQVHFCWLFFPFHRWYLYFYEKIMGELIGDPTFALPFWNRDAPAGMYIPEIFTDTSSSLYDQNRNTAHQPPKLLDLNYGGTDDDVDDATRIKENLTTMYQQMVSKATSHRLFYGEPYSAGDEPDPGAGNIETTPHNNIHLWVGDPTQTNGEDMGTFYSAGRDPLFYSHHSNVDRMWSIYKDKLGGTDIENTDWLDAEFLFYDEKKNLVRVKVRDSLDTKKLGYVYDEKVPIPWLKSKPTLVSRRIRERPQFIFDLTTTFPATLSDTISVEVTRPSATKRTAAQKKAHDEVLVIKGIEFAGNEPVKFDVYVNDDAESLAGKDKSEFAGSFVHVPHKHKKNIKTNLRLSIMSLLEELDAETDSSLVVTLVPKVGKGPITIGGFSIELINTT

SEQ ID NO: 2 - protein, 616 aa, Malus domestica
MASMSAPLVTSATSIIPTTSLSPFSQKYHRISLFGNPRHSNLQAVSCKATNNSSDQNKNPSTSSNDHDHENPSPVNLDRRNVLIGLGSLYGGVAGLGSDPFAVAKPVSPPDLAKCGAADFPSGAVPTNCCPPTSQKIVDFKFPSPTKLRVRPAAHTVDKAYIEKYSKAIELMKALPDDDPRSFTQQADIHCAYCDGAYDQVGFPNLELQIHQCWLFFPFHRYYLYFHERILAKLIYDPTFALPFWNWDAPAGMQLPALFANPDSPLYDELRAASHQPPTLIDLDFNGTDETMSNDAQIEANRKIMYRQMVSNSKKPLLFFGSPYRAGTEPDPGGGSIETTPHGPVHLWTGDNTQPNFEDMGNFYSAGRDPIFFSHHSNIDRMWNIWKSIGTKNKDINDKDWLDTGFLFYDENAELVRVTVRDTLDNKKLGYTYEDVEIPWLKSRPTPRRTKLARKAKAAGVAKAAGVAKAAETTSSGKVVAGKDFPINLETKISTVVSRPKPKKRSKKEKEDEEEILVIQGIELDKDVAVKFDVYVNDVDDEDAAPSGPDKSEFAGSFVSVPHKQKEKSKSCLRLGLTDLLEDLGAEDDESVVVTLVPRYGAQAVKIGSIKIEFLA

SEQ ID NO: 3 - protein, 592 aa, Artificial Sequence
  PPO peptide sequence from the arctic apple
MTSLSPPVVTTPTVPNPATKPLSPFSQNNSQVSLLTKPKRSFARKVSCKATNNDQNDQAQSKLDRRNVLLGLGGLYGVAGMGTDPFAFAKPIAPPDVSKCGPADLPQGAVPTNCCPPPSTKIIDFKLPAPAKLRIRPPAHAVDQAYRDKYYKAMELMKALPDDDPRSFKQQAAVHCAYCDGAYDQVGFPELELQIHNSWLFFPFHRYYLYFFEKILGKLINDPTFALPFWNWDSPAGMPLPAIYADPKSPLYDKLRSANHQPPTLVDLDYNGTEDNVSKETTINANLKIMYRQMVSNSKNAKLFFGNPYRAGDEPDPGGGSIEGTPHAPVHLWTGDNTQPNFEDMGNFYSAGRDPIFFAHHSNVDRMWSIWKTLGGKRTDLTDSDWLDSGFLFYNENAELVRVKVRDCLETKNLGYVYQDVDIPWLSSKPTPRRAKVALSKVAKKLGVAHAAVASSSKVVAGTEFPISLGSKISTVVKRPKQKKRSKKAKEDEEEILVIEGIEFDRDVAVKFDVYVNDVDDLPSGPDKTEFAGSFVSVPHSHKHKKKMNTILRLGLTDLLEEIEAEDDDSVVVTLVPKFGAVKIGGIKIEFA

SEQ ID NO: 4 - protein, 588 aa, Artificial Sequence
  PPO peptide sequence from the arctic apple
TTTTMGTYSSLIISTNSFSAFLPNKSQLSFSGKSKHYIARRSSIYCKATNPNYNNEQDQQQSSNLLGKLDRRNVLIGLGGLYGATTLAPKPLAFANPLAPPDLTKCKPAEITTGGETVECCPPVSTKIKTFTPDQSIPLRTRPAAHLVTDEYLAKFKKAQALMRALPEDDPRSMVQQAKVHCAYCNGAYPQVGFPDIDIQIHFSWLFFPFHRMYLYFYERILGKLIDDPTFALPYWNWDSPDGFPIPDIYTDTTSPLYDQYRNADHQPPVLVDLSYGGTDDDVDDQTRIDENLAIMYRQMVSGAKTPHLFFGHEYRAGDTTTGTYAGTIENSPHNNIHLWCGDPNQTHHEDMGNFYSAGRIPVYAHHCSDRMWNVWKTLGGKRKDPTDTDWLDAEFLFYDENAELVSCKVRDSLKPEKDLRYTYEPVSVPWLFTKPTARKPKSKTKAKVGATQLTTKFPATFDSKTTVEVARPKPRKRTKKEKIDEEEVLIIKDIEFESNEAVKFDVFINDDAESLSRKDKSEFAGSFVHVPHNQKTGTKKKTNLKLGITDLLEDLGVEDDSSVLVTLVPRVSNSPITIGGFKIEYSS

SEQ ID NO: 5 - DNA, 1734 bp, Musa acuminata
atggcttctatctcgcagctaatcactacaagcatccccaccaccttttctctctcctattcatgccccttccctccgagaaccacggtatcgatctccggcttcaaacacctccaccacgtttcccccatctcatgctccaccagagaccacaaccaacccctcgtcgatcgccgccacgtcctcgtcggaataggcagcctctacggcgcctccgctgcactaacgtccctccgcgaggccagtgcggcaccgatcgcggcgcccgacctgtccgcatgcgggctggctgacctccctccggatgccactccgacgaactgctgcccgccgtccgcgggagacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttcttccagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcagtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggctgatgggagcatcatggtgactttggtgccgaggcaggggaaggggaaggtgaaggttggcagtctcaagatcgagttagttgat

SEQ ID NO: 6 - DNA, 1761 bp, Musa acuminata
atggccggccttccttattcagctcctcaccctgccaccatctccgcttcctccaactcctttgcatgccccttccgcagcaaggggcttgtcttcccctaccctaccagaagagcactccatgttcgtcccaacatcgcatgcaaggcaggcgaggagcacgagatcgctgctaaggtcgaccgacgcgacgtactcgtgggcctcggtgggctctgcggagccgccgctggccttggcgggttcgataaagccgccctcgctaaccccattcaggcccctgatctctccaagtgcggccctgccgacctccccaccggcgtgccagtcgtcaactgctgcccgccctaccgtcccggtgcgaagattgtggatttcaagcggccgtcgccgtcctccccactccgcgtccgccccgccgcccacttggttgaccccgagtacctggccaagtacaagaaggccatcgagctcatgaaggcgctcccggccgatgaccctcgcaacttcatgcagcaggccgacgtccactgcgcctactgcgacggcgcttacgaccagatcggcttccccaaccttgagatccaagtccacaacagctggctcttcttcccctggcaccgcttgtacctctacttcaacgagaggatcctcggcaagctcatcggcgacgacaccttctcgctccctttctggaactgggacgcacccggcggaatgatgctgccttcgatctacgccgatccttcgtcacccctctacgacaaacttcgcgacgccaagcaccaacctcctgtccttgtcgacctcgactacaatggaaccgacccaaccttccccgacgcccagcaaatcgatcacaacctcaagatcatgtaccgccaagtcttctccaacggcaagacgccgttgctgttcttaggctcagcttaccgtgccggtgaacagcctaaccctggcgcgggctccgtcgagaacatgccgcacaacaacgtgcacttgtggaccggcgaccgcacccagcccaacttcgagaacatgggcaccttctacgccgcggcgcgcgaccccatcttcttcgcccaccacgccaacatcgaccgcatgtggtacctgtggaagaagctcagcaggaagcaccaggacttcaatgactcggactggctcaaagcttccttccttttctacgacgagaacgccgacttagttcgggtcacggtcaaggactgcttggagaccgattggctgcgctacacgtaccaagacgtgaagatcccatgggtgaacgcccgaccgactcccaagctcgccaaggcgaggaaagccgccagcagttcgctgaaagccaccgcggaggtgcagttccctgtgacgctggaatccccggtcaaagcgacggtgaagaggcccaaggtggggaggagcggcaaggagaaggaagatgaggaggagatactcatagtggaggggatcgagttcgaccgcgactacttcatcaagttcgacgtcttcgtgaacgcgacggagggcgacggcatcacggccggggccagcgagttcgccggcagcttcgtgaacgtcccgcacaagcacaagcaccgcaaggatgagaataagctgaagacgaggctgtgtcttggaatcaccgacctgctcgaggacatcggcgcggaggacgacgacagcgtgctcgtcaccatcgtgccgaaggcgggcaaaggaaaggtgtccgtcggcggtcttcggattgacttttccaag

SEQ ID NO: 7 - DNA, 1608 bp, Musa acuminata
atggcagagatcggcaatccaaatgaaaacaacacctcaatatttgccgaccgcaagccgccgaaacgcctcgtgttgccattcggcgtccgaccaccagtggcagaggcactgacaaaggttgatgcaagtttctgcgaccccaagaacaacaatgagtgtataaacttaggatcccaaggaggcgagtgcgccgccaactgctgccttcccatccctcccggtgcgaagattgtggatttcaagcggccatcgcggtccacccccctccgcgtccgccccgccgcccacttggtcgaccccgagtacctggccaagtacaagaaggccatcgagctcatgaaggcgctcccggccgacgaccctcgcaacttcatgcagcagtccaacgtccactgcgttcactgcgacagcatccccgacaatgacatccaagtccaccagaactggttcttctacccctggcaccgctggtacctctacttcaacgagaggatcctcggcaagctcatcggcgacgacaacttcacgctccctttctggaactgggactcgctcggcggaatgatgctgccgtcgatctacgccgacccttcgtcgcccctctacgacaaccttcgcgacgccaagcaccaacctcctttccttgtcgacttcgacttcaatggaaccgacccaggcttcaccgacgcccagcaaatcgatcacaacctcaagatcatgtaccgccaattcttctccaacggcaagaagccgatgctgttcttaggctcaccttaccgcgggggcgacaagcctaaccccggcgggggctccgtcgagaacacgccgcacaacaacgtgcacacgtggaccggcgaccgtactcgtcccaacttcgaggacatgggcaccttctactcggcggggcgcgaccccatcttcttcgcccaccacgccaacatcgatcgcatgtggtccctgtggaagaagctcagccgtaagcaccgggacttcaatgactcggactggctgaaaacttccttcctcttctacgacgagaacgccgacttagttcgggtcacggtcaaggactgcttcaagacccggtggctgcgctacaagtaccaagacgtggagatcccatgggtgaaagcccgaccgactcccaagctcaccaaggcgaggaaagccgccagcggatcgctgaaacccaccgcggaggcgcagttccctgtgacgctggaatccccggtcagcgcgacgctgaagaggcccaaggtggggaggagccgcaaggagaaggaagaggaggaggaagtgctcatagtggaggggatcgagttcgaccgcgaccagttcatcaagttcgacgtcatcgtgaacgcgacggagggcgatggcatcacgcccgcggacagcgagttcgccggcagcttcgtgaacaccccgcacaggcacaggcacctcaaggaggagaacaaggggacgacgaggctgtgtctggggatcaccgacctgctcgaggacatcggcggggaggccgacgacggcgtgctcgtcaccatcgtacccaaggcaggcaagggcaaggtttccgtcggcggtcttcggattgacttcaccaag

SEQ ID NO: 8 - DNA, 1770 bp, Musa acuminata
atgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgccgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgaccccggcgagacgatctcggagtacagcttccccgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaataccgacgacgtgcccatccaggtccacttcagctggatcttcctcccatggcaccgctactacctccacttctacgaaaggatcctcggcaagctcatcgacgacgacaccttcaccatcccattctggaactgggacaccaaggacggcatgacgttccccgccatcttccaggatgcggcatccccgctgtacgacccgaaacgcgaccaacgccacgtcaaggacggcaagatcctcgacctcaagtacgcctacaccgaaaacactgcatccgacagcgagatcatacgggagaacctctgcttcatacagaagacgttcaagcacagcctgtcgctggcggaactgttcatgggggatcccgtgcgcgcgggggagaaggagatccaggaggctaatgggcagatggaagtcatccacaatgcggcgcacatgtgggtcggagagccggacggatacaaggaaaacatgggggacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgaacagctcgtcaaagtcaagatgagcgactgcctcaacccgaccaagcttcggtacacgttcgaacaagttcccctcccatggctgggcaaaatcaattgccagaagacggcagagacgaagtccaaggccacgacggagctgtcgctgacgcgcgtgaacgaattcgggacgacggcccaggcactcgacgcgagcaacccgctgcgggtgatcgtggcaaggccgaagaagaaccgcaagaagaaggagaagcaagagaaggtggaggtgattcagatcaaggatattcaggtgaccaccaacgagacagctcgcttcgacgtctacgtcgcggttccttacggtgacctcgccggacccgactacggcgagttcgcgggcagctacgtgaggctggcgcataggatgaagggaagcgacgggaccgcagagcaggtccccaagaagaagggaaagctcaagctgggtattacgccgctgctcgaggacatcgatgctgaggacgccgacaagttggtggtcaccctggttctccgcaccgggagcgtcaccgtgggcggagtttccatcaatctcctgcagacagattctaccgccgccatc

SEQ ID NO: 9 - DNA, 1524 bp, Musa acuminata
atgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgctgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgtccccggcgagacgatctcggagtacagcttccctgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaatgccgacgacgtgcccatccaggtccacttcagctggatcttcctcccatggcaccgctactacctccacttctacgaaaggatcctcggcaagctcatcgacgacgacaccttcaccatccccttctggaactgggacaccaaggacggcatgacgttccccgccatcttccaggatgcggcatccccgctgtacgacccgaaacgcgaccaacgccacgtcaaggacggcaagatcctcgacctcaagtacgccatcaccgaaaatgaaagcactgcatccgacagcgagatcatacgggagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacagttgggtcggagagccgagcggaaactatgaagacatgggcgacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgagcagctcgtcaaagtcaagatgagggactgcctcaacccgaccaagcttcggtacacgttcgagcaagttcccctcccatggctgggcaaaatcaattgccagaagacggcagagacgaagtccaaggccacgacggagctgtcgctgaatcgcgtgaacgaattcggaacgacggcccaggcactcgacgcgagcaacccgctgcgggtgatcgtggcaaggccgaagaagaaccgcaagaagaaggagaagcaagagaaggtggaggtgattcagatcaaggatattaaggtgaccaccaacgagacagctcgcttcgacgtctacgtcgcggtcccttacggtgacctc

SEQ ID NO: 10 - DNA, 1776 bp, Musa acuminata
atgtctctcctgttgaactctagcctcaccggagcttcctctgcatgcctcctccgtcgagaaaagtgccgccgccgcggccgcggtcacgtccacggcgtgacatgccaccaggggggtaatgatgaccgcagagaggccgcccggcagcagcggtcccggttgctgctggatcggcgcgacatgctgttgggggggttaggagggctttacggcgtgaccgcagggcccaaagttctggcggagccgataatgccgcctgatctgtccaagtgccacgatgccaacgcacctgccctcgacaaccactgctgcccgccttacagtggcagcgagacgatcttggagtacgacttccccgctacgcccctccgggtgcggcgaccggcccacctcgtgaaggatgatcaggagtatatggacaagtacaaggaggccgtgaggaggatgaagaacctgccggcagaacacccttggaactactaccagcaggcgaacatccactgccagtattgcaacgacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccaggatgcggcatccccgctgtacgacccgaaacgcgaccaacgtcacgtcaaggacggcgcgatcctcgacctcaagtacgcctacaccgaaaacactgcatccgacagcgagatcatacgggagaacctctgcttcatacaaaagacgttcaagcacagcctgtcgctggcggagctgttcatgggggatcccgtgcgcgcgggggagaaggagatccaggaggcaaacgggcagctggaagtcatccacaatgcggcgcacatgtgggtcggagagccggacggatacaaggaaaacatgggcgacttctccaccgcggcccgcgattctgttttcttctgccaccattccaatgtcgaccgcatgtgggacatctaccgcaacctccgcggcaacagcgtcgagttcaacgacaaagactggttgcacagcaccttcctcttccatgacgagaacgagcagctcgtcaaagtcaagatccaggactgccttaacccgaccaagcttcggtacacgttcgagcaagttcccctcccatggctgggcaatataaattgccagaagacggcagagacgaagtccaagtccacggcagagctgtcgctgaagcgggtgggcgaattcgggacgacacccaaggcgctcgacgcgagcaacccgctgcgggtgatcgtggcaaggccgaagaagaaccgcaagaagaaggagaagcaagagaaggtggaggtgctccagatcaaggatattaaggtgaccaccaacgagacagctcgcttcgacgtctacgtcgccgttccttacggtgacctcgccggacccgactacggcgagttcgtgggcagcttcgttaggctggcgcataggaagaagggaagcgacgggaccgaagagcagggccccaagaagaagggaaagctcaagctgggtattacggcgctgctcgaggacatcgatgctgaggacgccgacaagttggtggtcaccctggttctccgcaccgggagcgtcactgtgggtggagtttccatcaaactcctgcagacagatactcccgccgtcatc

SEQ ID NO: 11 - DNA, 1512 bp, Musa acuminata
atggctctccagttgaactctagcttcaccggagcttcctctgcatgcctcctccatcgggaaaggtcccgccgcctcaacgtccctgtcgtgacatgccgccaggggaataatgatgatcgcagcgatgccgctcgccagcagaaatccccgtcactactggatcggcgcgacatgctgctggggttaggagggctttatggcttgaccgcaggacccaaagttctggcgaagccgataatgccgcctgatctgtccaagtgccacgatgccaaggcacctgccctcgacaaccactgctgcccgccttacaaccccagcgagacgatctcggagtacggcttccccgctacgcccctccgggtgcggcggccggcccacctcgtgaaggacgatcaggagtatttggacaagtacaaggaggccgtgaggaggatgaagaatctgccggcagaccacccttggaactactaccagcaggcgaacgtccactgccagtactgcaactacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccacgaagagtcatccccgctgtctgacacgaaacgcgaccaacgccacgtcaaggatggcaagatcgtcgacctcaagtacgcctacaccgaaaaccctgcctccaacagcgagatcattcgagagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacatgtgggtcggagagccgtgcggatacaaggaaaacatgggcgatttctctaccgccgcccgcgattctgttttcttcagccaccactccaatgtcgaccgcttgtgggaaatctaccggaacctccgcggtaaccgcattgagttcgaagacaacgactggttggacagtaccttcctcttctacgacgagaacgagaagctcgtcaaagtcaagatgggggactgcctcaacccgaccaagcttcggtatacgttcgagcaagttcctctcccatggctgggcaaaattaattgccagaagacgacagagacgaagtccaagtccacgacagagatgtcgctgacgcgcgtgggagaattcgggacgacgcccaaggcgctcgacgcgagcaacccgctgcgggtgatcgtggcaaggccgaaaaagaaccgcaagaagaaggagaagcaagagaaggtggaggtgcttcagatcaatgatattaaggtgaccaccaacgagacagctcgcttcgacgtctacgtcacggttccttacggtgacctc

SEQ ID NO: 12 - DNA, 1770 bp, Musa acuminata
atggtcagccttcctaaagctactcttcctctctcctccctctcccctccctccaactccaactccaactccaactccaactcctttgcatgcgccttccatttttcttaccctgatagaagacgccatgcccactccaagatctcatgcaaagctagcgatgagcatgagatgactgcaaatgccaagctcgaccgccgcgatgtgctcgttggcctcggcgggctttgtggagccgctgccggcctcgggatcgacggtaaggccctcggaaatcccatccaggcgcctgatcttaccaagtgcggccccgccgatctacccaaaggtgcaacgcccaccaactgctgcccgccttattttcccgacaagaagattatcgatttcaagcgtccgccgaattcgtcacccctccgtgtccgcccggccgcccacttggtcgactccgactacctggacaagtataagaaggcggtggagctcatgagagcactgccggccgacgatccgcgcaacttcatgcagcaggccaatgtccactgcgcttactgtgatggcgcctacgaccagatcggcttccccaacctcgagctccaagtccacaactcctggctcttcttcccttggcaccgcttctacctctacttccacgagaggatcctcggcaagctcataggcgacgacactttcgcccttcctttctggaactgggacgcgcccggcggcatgaagctgccgtcgatctacgccgatccttcgtcctcgctctatgacaagtttcgcgacgccaagcaccagccgccggtcctcgtcgacctcgactacaacggaaccgaccctagtttcaccgacgcagagcagatcgatcagaacctcaagatcatgtaccggcaggtgatctccaacggcaagacgccgttgctgttcttaggctcggcttaccgtgccggcgacaacccaaaccccggcgcgggctcgctcgagaacataccacacggccccgtccacgggtggactggcgacagaaaccaacccaatctcgaggacatgggcaacttctactccgcggggcgcgaccctatcttcttcgcccaccattcaaacgtcgaccgcatgtggtacttgtggaagaagctcggcgggaagcatcaggactttaacgataaggactggctcaacaccaccttcctcttctacgacgagaatgctgacttagttcgagtcaccctcaaggactgcttgcagccggagtggcttcgttacgattaccaagacgtcgagatcccgtggctgaagacccggccgactcccaaagccttgaaggcgcagaaaaccgcagcgaaaacactgaaagctacagcagagacgccgttcccggtgacgctgcaatccgcggtgagcacgacggtgaggaggcccaaggtatcgaggagcggcaaggagaaggaagaggaagaggaggtcctcatcgtggaggggatcgagttcgaccgcgactacttcataaagttcgacgtcttcgtgaacgccaccgagggtgagggcatcacgccgggcgccagcgagttcgcgggcagcttcgtcaacgtcccgcacaagcacaagcacagcaagaaggagaagaagctgaagacgaggctctgcctggggatcactgacctgctcgaggacatcggggcggaggacgacgacagcgtgctcgtcaccatcgtcccgaaagccggaaagggcaaggtgtcggtcgccggcctccgcatcgatttcccaaat

SEQ ID NO: 13 - DNA, 1722 bp, Musa acuminata
atggagggcaaacgatggttgtctcttctcctcctcgtgcttgttctcgtgggcatctccatggatctcccgagagaagctccggcggcttcttctaatatcttgaagagttcatctgccagaataccagtgaatcctcaaggcggagaacagagagatggaagcaagagcaagggcattcccctcaaagcaaacctatcggtttgccatgcttcattctcggacgccgatcgtccggtctactgttgcccggcctggaaagacgccgaccaaaccttgctcgacttcgagttcccggatccgtcgtcgccggtgcgtatccgacggcctgcccatctcgtcgacgaggagttcgtggccaagtatgagagggcggtggccatcatgaagcagatcccgcctgaccatccccacaacttttggcgccaggccaacatgcactgcctctactgcaccggcgcctacgaccagatgaactcctctgccctcttcaagatccacaggtcatggctcttcttcccctggcaccgagccttcatctatttccacgagcgcatcctcgggaagttcatgggagacgacaccttcgcgctcccctactggagctgggacacccccgagggcatgtggttccccgacatctaccggaagggagctctgaatgagacggagcgcgacgccattcacctacgggaggccgccgtcgatgacttcgactacgtggatcatgacctcgccagcgacgtgcagatcgccgacaacctcgcgttcatgtaccaccagatgatctcgggagcgaagaagaccgagctgttcatgggttgcaagctgcggtccggcgtcgaggggtggtgtgatgggcccgggacgatcgaagcggcacctcacaacacgttgcacagctgggtagggaacaggtacaaccccgaaagagagaacatgggggcgttctactccgccgcgcgagacgaagtgttcttcgcgcaccactccaacatcgaccgcatgtggaccgtgtggaagaagctgcacggcgacaagccggagttcgtcgaccaggagtggctcgagtcggagttcaccttctacgacgagaatgtgcgcctgcgcaggatcaaggtgcgcgacgtgttgaacatagacaaactcaggtaccggtacgaagacatcgacatgccatggctcgctgcacgtcccaagccttccgttcaccctaagatcgcgcgcgacatattgaagaagcgcaatggcgaaggcgtactgagaatgcccggcgaaacggatcgttcacaactctccgaatatggtagctggacactggacaagaccatcaccgtgagggttgacaggccaaggatcaacaggacagggcaagaaaaagaggaagaagaggagatcttattggtctacggaatcgatactaagagaagcagattcgtcaaattcgatgtgttcatcaacgtcgtcgacgaaaccgtgctgagcccaaagtcgagggagttcgcagggaccttcgtcaatctccaccacgtctcgaggacgaaaagccatgacgatggcggcatggattcgaagatgaaaagccaccttaagctcggtatatcggaacttttggaagacctcgaggcagacgaagatgacagcatctgggtgacactggtgccaagaggcggcacgggggtcaacaccaccgtggacggcgtccggatcgactacatgaag

SEQ ID NO: 14 - DNA, 20 bp, Artificial Sequence
  Forward oligonucleotide used to identify the expression of mRNA-encoding PPO1
cagcttcgcgattccgttct

SEQ ID NO: 15 - DNA, 20 bp, Artificial Sequence
  Reverse oligonucleotide used to identify the expression of mRNA-encoding PPO1
ttccagatgcggtcgatgtt

SEQ ID NO: 16 - DNA, 20 bp, Artificial Sequence
  Forward oligonucleotide used to identify the expression of mRNA-encoding PPO2
gagcactccatgttcgtccc

SEQ ID NO: 17 - DNA, 20 bp, Artificial Sequence
  Reverse oligonucleotide used to identify the expression of mRNA-encoding PPO2
ggaagccgatctggtcgtaa

SEQ ID NO: 18 - DNA, 20 bp, Artificial Sequence
  Forward oligonucleotide used to identify the expression of mRNA-encoding PPO3
aagtttctgcgaccccaaga

SEQ ID NO: 19 - DNA, 20 bp, Artificial Sequence
  Reverse oligonucleotide used to identify the expression of mRNA-encoding PPO3
cagttccagaaagggagcgt

SEQ ID NO: 20 - DNA, 21 bp, Artificial Sequence
  Forward oligonucleotide used to identify the expression of mRNA-encoding PPO4
gagttcgaagacaacgactgg

SEQ ID NO: 21 - DNA, 22 bp, Artificial Sequence
  Reverse oligonucleotide used to identify the expression of mRNA-encoding PPO4
gtcgcttcccttcatcctatgc

SEQ ID NO: 22 - DNA, 20 bp, Artificial Sequence
  Forward oligonucleotide used to identify the expression of mRNA-encoding PPO5
gagccgagcggaaactatga

SEQ ID NO: 23 - DNA, 20 bp, Artificial Sequence
  Reverse oligonucleotide used to identify the expression of mRNA-encoding PPO5
gtctcgttggtggtcacctt

SEQ ID NO: 24 - DNA, 20 bp, Artificial Sequence
  Forward oligonucleotide used to identify the expression of mRNA-encoding PPO6
accgaaaacactgcatccga

SEQ ID NO: 25 - DNA, 19 bp, Artificial Sequence
  Reverse oligonucleotide used to identify the expression of mRNA-encoding PPO6
cgggttaaggcagtcctgg

SEQ ID NO: 26 - DNA, 20 bp, Artificial Sequence
  Forward oligonucleotide used to identify the expression of mRNA-encoding PPO7
aaaaccctgcctccaacagc

SEQ ID NO: 27 - DNA, 21 bp, Artificial Sequence
  Reverse oligonucleotide used to identify the expression of mRNA-encoding PPO7
ggacttcgtctctgtcgtctt

SEQ ID NO: 28 - DNA, 20 bp, Artificial Sequence
  Forward oligonucleotide used to identify the expression of mRNA-encoding PPO8
gatagaagacgccatgccca

SEQ ID NO: 29 - DNA, 20 bp, Artificial Sequence
  Reverse oligonucleotide used to identify the expression of mRNA-encoding PPO8
gaagccgatctggtcgtagg

SEQ ID NO: 30 - DNA, 19 bp, Artificial Sequence
  Forward oligonucleotide used to identify the expression of mRNA-encoding PPO9
tgcttgttctcgtgggcat

SEQ ID NO: 31 - DNA, 20 bp, Artificial Sequence
  Reverse oligonucleotide used to identify the expression of mRNA-encoding PPO9
gaagagggcagaggagttca

SEQ ID NO: 32 - DNA, 19 bp, Artificial Sequence
  Variable sequence of the PPO1 sgRNA1 sg2019
agggacgttagtgcagcgg

SEQ ID NO: 33 - DNA, 20 bp, Artificial Sequence
  Variable sequence of the PPO1 sgRNA2 sg858
cctcaagggcgaggacgggt

SEQ ID NO: 34 - DNA, 20 bp, Artificial Sequence
  Variable sequence of the PPO2 sgRNA1 sg854
aggaagcggagatggtggca

SEQ ID NO: 35 - DNA, 20 bp, Artificial Sequence
  Variable sequence of the PPO2 sgRNA2 sg855
atgcgatgttgggacgaaca

SEQ ID NO: 36 - DNA, 20 bp, Artificial Sequence
  Variable sequence of the PPO3 sgRNA1 sg1435
ggttaggcttgtcgcccccg

SEQ ID NO: 37 - DNA, 20 bp, Artificial Sequence
  Variable sequence of the PPO3 sgRNA2 sg1436
aggactgcttcaagacccgg

SEQ ID NO: 38 - DNA, 20 bp, Artificial Sequence
  Variable sequence of the PPO9 sgRNA1 sg850
aaccgataggtttgctttga

SEQ ID NO: 39 - DNA, 20 bp, Artificial Sequence
  Variable sequence of the PPO9 sgRNA2 sg851
ggtttggtcggcgtctttcc

SEQ ID NO: 40 - protein, 578 aa, Musa acuminata
MASISQLITTSIPTTFSLSYSCPFPPRTTVSISGFKHLHHVSPISCSTRDHNQPLVDRRHVLVGIGSLYGASAALTSLREASAAPIAAPDLSACGLADLPPDATPTNCCPPSAGDATEFVIPDPSSPLRVRPAAHSVDKDYIAKFAKGVALMKALPADDPRNFTQHANVHCAYCDGAYSQVGFPDLELQVHNSWLFLPWHRCYLYFFERILGKLIGDDSFAIPFWNWDAPDGMRLPAMYVDPTSPLYDPLRDAQHQPPTLVDLDFGGIDPSSSDKQQIDHNLKVMYRQIVSNAPTPRLFFGNPYRAGDNPNPGGGSLENVPHGPVHVWTGDRSQSELEDMGNLYSAARDPVFFAHHSNIDRIWNVWKGLGSRRKDLADPDWLDASFVFYDENANLVKIRVRDCIDSDKLRYEYQDVGNPWLNTRPTVTSGVRPRVAGVAHANVVEPKFPIKLDSVVTAKVKRPKAARTKEEKEEKEEVLVVEGIELDRDVHVKFDVFVNVTDHGKVGPGGRELAGSFVNVPHRHKHDKMSKQLKTRLQLGLTELLEDLKADGSIMVTLVPRQGKGKVKVGSLKIELVD

SEQ ID NO: 41 - protein, 587 aa, Musa acuminata
MAGLPYSAPHPATISASSNSFACPFRSKGLVFPYPTRRALHVRPNIACKAGEEHEIAAKVDRRDVLVGLGGLCGAAAGLGGFDKAALANPIQAPDLSKCGPADLPTGVPVVNCCPPYRPGAKIVDFKRPSPSSPLRVRPAAHLVDPEYLAKYKKAIELMKALPADDPRNFMQQADVHCAYCDGAYDQIGFPNLEIQVHNSWLFFPWHRLYLYFNERILGKLIGDDTFSLPFWNWDAPGGMMLPSIYADPSSPLYDKLRDAKHQPPVLVDLDYNGTDPTFPDAQQIDHNLKIMYRQVFSNGKTPLLFLGSAYRAGEQPNPGAGSVENMPHNNVHLWTGDRTQPNFENMGTFYAAARDPIFFAHHANIDRMWYLWKKLSRKHQDFNDSDWLKASFLFYDENADLVRVTVKDCLETDWLRYTYQDVKIPWVNARPTPKLAKARKAASSSLKATAEVQFPVTLESPVKATVKRPKVGRSGKEKEDEEEILIVEGIEFDRDYFIKFDVFVNATEGDGITAGASEFAGSFVNVPHKHKHRKDENKLKTRLCLGITDLLEDIGAEDDDSVLVTIVPKAGKGKVSVGGLRIDFSK

SEQ ID NO: 42 - protein, 536 aa, Musa acuminata
MAEIGNPNENNTSIFADRKPPKRLVLPFGVRPPVAEALTKVDASFCDPKNNNECINLGSQGGECAANCCLPIPPGAKIVDFKRPSRSTPLRVRPAAHLVDPEYLAKYKKAIELMKALPADDPRNFMQQSNVHCVHCDSIPDNDIQVHQNWFFYPWHRWYLYFNERILGKLIGDDNFTLPFWNWDSLGGMMLPSIYADPSSPLYDNLRDAKHQPPFLVDFDFNGTDPGFTDAQQIDHNLKIMYRQFFSNGKKPMLFLGSPYRGGDKPNPGGGSVENTPHNNVHTWTGDRTRPNFEDMGTFYSAGRDPIFFAHHANIDRMWSLWKKLSRKHRDFNDSDWLKTSFLFYDENADLVRVTVKDCFKTRWLRYKYQDVEIPWVKARPTPKLTKARKAASGSLKPTAEAQFPVTLESPVSATLKRPKVGRSRKEKEEEEEVLIVEGIEFDRDQFIKFDVIVNATEGDGITPADSEFAGSFVNTPHRHRHLKEENKGTTRLCLGITDLLEDIGGEADDGVLVTIVPKAGKGKVSVGGLRIDFTK

SEQ ID NO: 43 - protein, 590 aa, Musa acuminata
MSLLLNSSFTGASSACLLQRERSRRRRLHVPGVTCRQGSNGDRSDAARQQQSPPLLDRRDMLLGLGGLYGVTAGPKVLAAPIMPPDLSKCYPATAPALDNKCCPPYDPGETISEYSFPATPLRVRRPAHIVKDDQEYMDKYKEAVRRMKNLPADHPWNYYQQANIHCQYCNYAYHQQNTDDVPIQVHFSWIFLPWHRYYLHFYERILGKLIDDDTFTIPFWNWDTKDGMTFPAIFQDAASPLYDPKRDQRHVKDGKILDLKYAYTENTASDSEIIRENLCFIQKTFKHSLSLAELFMGDPVRAGEKEIQEANGQMEVIHNAAHMWVGEPDGYKENMGDFSTAARDSVFFCHHCNVDRMWDIYRNLRGNRVEFEDNDWLDSTFLFHDENEQLVKVKMSDCLNPTKLRYTFEQVPLPWLGKINCQKTAETKSKATTELSLTRVNEFGTTAQALDASNPLRVIVARPKKNRKKKEKQEKVEVIQIKDIQVTTNETARFDVYVAVPYGDLAGPDYGEFAGSYVRLAHRMKGSDGTAEQVPKKKGKLKLGITPLLEDIDAEDADKLVVTLVLRTGSVTVGGVSINLLQTDSTAAI

SEQ ID NO: 44 - protein, 508 aa, Musa acuminata
MSLLLNSSFTGASSACLLQRERSRRRRLHVPGVTCRQGSNGDRSDAARQQQSPLLLDRRDMLLGLGGLYGVTAGPKVLAAPIMPPDLSKCYPATAPALDNKCCPPYVPGETISEYSFPATPLRVRRPAHIVKDDQEYMDKYKEAVRRMKNLPADHPWNYYQQANIHCQYCNYAYHQQNADDVPIQVHFSWIFLPWHRYYLHFYERILGKLIDDDTFTIPFWNWDTKDGMTFPAIFQDAASPLYDPKRDQRHVKDGKILDLKYAITENESTASDSEIIRENLCFIQKTFKHSLSLAELFMGDPVRAGEKEIQEANGQLEVIHNAVHSWVGEPSGNYEDMGDFSTAARDSVFFCHHCNVDRMWDIYRNLRGNRVEFEDNDWLDSTFLFHDENEQLVKVKMRDCLNPTKLRYTFEQVPLPWLGKINCQKTAETKSKATTELSLNRVNEFGTTAQALDASNPLRVIVARPKKNRKKKEKQEKVEVIQIKDIKVTTNETARFDVYVAVPYGDL

SEQ ID NO: 45 - protein, 592 aa, Musa acuminata
MSLLLNSSLTGASSACLLRREKCRRRGRGHVHGVTCHQGGNDDRREAARQQRSRLLLDRRDMLLGGLGGLYGVTAGPKVLAEPIMPPDLSKCHDANAPALDNHCCPPYSGSETILEYDFPATPLRVRRPAHLVKDDQEYMDKYKEAVRRMKNLPAEHPWNYYQQANIHCQYCNDAYYQQNTDDVPVQVHFSWIFLPWHRYYLHFYERILGKLIDDDTFTIPFWNWDTKDGMTFPAIFQDAASPLYDPKRDQRHVKDGAILDLKYAYTENTASDSEIIRENLCFIQKTFKHSLSLAELFMGDPVRAGEKEIQEANGQLEVIHNAAHMWVGEPDGYKENMGDFSTAARDSVFFCHHSNVDRMWDIYRNLRGNSVEFNDKDWLHSTFLFHDENEQLVKVKIQDCLNPTKLRYTFEQVPLPWLGNINCQKTAETKSKSTAELSLKRVGEFGTTPKALDASNPLRVIVARPKKNRKKKEKQEKVEVLQIKDIKVTTNETARFDVYVAVPYGDLAGPDYGEFVGSFVRLAHRKKGSDGTEEQGPKKKGKLKLGITALLEDIDAEDADKLVVTLVLRTGSVTVGGVSIKLLQTDTPAVI

SEQ ID NO: 46 - protein, 504 aa, Musa acuminata
MALQLNSSFTGASSACLLHRERSRRLNVPVVTCRQGNNDDRSDAARQQKSPSLLDRRDMLLGLGGLYGLTAGPKVLAKPIMPPDLSKCHDAKAPALDNHCCPPYNPSETISEYGFPATPLRVRRPAHLVKDDQEYLDKYKEAVRRMKNLPADHPWNYYQQANVHCQYCNYAYYQQNTDDVPVQVHFSWIFLPWHRYYLHFYERILGKLIDDDTFTIPFWNWDTKDGMTFPAIFHEESSPLSDTKRDQRHVKDGKIVDLKYAYTENPASNSEIIRENLCFIQKTFKHSLSLAELFMGDPVRAGEKEIQEANGQLEVIHNAVHMWVGEPCGYKENMGDFSTAARDSVFFSHHSNVDRLWEIYRNLRGNRIEFEDNDWLDSTFLFYDENEKLVKVKMGDCLNPTKLRYTFEQVPLPWLGKINCQKTTETKSKSTTEMSLTRVGEFGTTPKALDASNPLRVIVARPKKNRKKKEKQEKVEVLQINDIKVTTNETARFDVYVTVPYGDL

SEQ ID NO: 47 - protein, 590 aa, Musa acuminata
MVSLPKATLPLSSLSPPSNSNSNSNSNSFACAFHFSYPDRRRHAHSKISCKASDEHEMTANAKLDRRDVLVGLGGLCGAAAGLGIDGKALGNPIQAPDLTKCGPADLPKGATPTNCCPPYFPDKKIIDFKRPPNSSPLRVRPAAHLVDSDYLDKYKKAVELMRALPADDPRNFMQQANVHCAYCDGAYDQIGFPNLELQVHNSWLFFPWHRFYLYFHERILGKLIGDDTFALPFWNWDAPGGMKLPSIYADPSSSLYDKFRDAKHQPPVLVDLDYNGTDPSFTDAEQIDQNLKIMYRQVISNGKTPLLFLGSAYRAGDNPNPGAGSLENIPHGPVHGWTGDRNQPNLEDMGNFYSAGRDPIFFAHHSNVDRMWYLWKKLGGKHQDFNDKDWLNTTFLFYDENADLVRVTLKDCLQPEWLRYDYQDVEIPWLKTRPTPKALKAQKTAAKTLKATAETPFPVTLQSAVSTTVRRPKVSRSGKEKEEEEEVLIVEGIEFDRDYFIKFDVFVNATEGEGITPGASEFAGSFVNVPHKHKHSKKEKKLKTRLCLGITDLLEDIGAEDDDSVLVTIVPKAGKGKVSVAGLRIDFPN

SEQ ID NO: 48 - protein, 574 aa, Musa acuminata
MEGKRWLSLLLLVLVLVGISMDLPREAPAASSNILKSSSARIPVNPQGGEQRDGSKSKGIPLKANLSVCHASFSDADRPVYCCPAWKDADQTLLDFEFPDPSSPVRIRRPAHLVDEEFVAKYERAVAIMKQIPPDHPHNFWRQANMHCLYCTGAYDQMNSSALFKIHRSWLFFPWHRAFIYFHERILGKFMGDDTFALPYWSWDTPEGMWFPDIYRKGALNETERDAIHLREAAVDDFDYVDHDLASDVQIADNLAFMYHQMISGAKKTELFMGCKLRSGVEGWCDGPGTIEAAPHNTLHSWVGNRYNPERENMGAFYSAARDEVFFAHHSNIDRMWTVWKKLHGDKPEFVDQEWLESEFTFYDENVRLRRIKVRDVLNIDKLRYRYEDIDMPWLAARPKPSVHPKIARDILKKRNGEGVLRMPGETDRSQLSEYGSWTLDKTITVRVDRPRINRTGQEKEEEEEILLVYGIDTKRSRFVKFDVFINVVDETVLSPKSREFAGTFVNLHHVSRTKSHDDGGMDSKMKSHLKLGISELLEDLEADEDDSIWVTLVPRGGTGVNTTVDGVRIDYMK

SEQ ID NO: 49 - DNA, 22 bp, Artificial Sequence
  Variable sequence of PPO1 sgRNA sg2019 including PAM site
agggacgttagtgcagcggagg

SEQ ID NO: 50 - DNA, 23 bp, Artificial Sequence
  Variable sequence of PPO1 sgRNA sg858 including PAM site
cctcaagggcgaggacgggtcgg

SEQ ID NO: 51 - DNA, 23 bp, Artificial Sequence
  Variable sequence of PPO2 sgRNA sg854 including PAM site
aggaagcggagatggtggcaggg

SEQ ID NO: 52 - DNA, 23 bp, Artificial Sequence
  Variable sequence of PPO2 sgRNA sg855 including PAM site
atgcgatgttgggacgaacatgg

SEQ ID NO: 53 - DNA, 23 bp, Artificial Sequence
  Variable sequence of PPO3 sgRNA sg1435 including PAM site
ggttaggcttgtcgcccccgcgg

SEQ ID NO: 54 - DNA, 23 bp, Artificial Sequence
  Variable sequence of PPO3 sgRNA sg1436 including PAM site
aggactgcttcaagacccggtgg

SEQ ID NO: 55 - DNA, 23 bp, Artificial Sequence
  Variable sequence of PPO9 sgRNA sg850 including PAM site
aaccgataggtttgctttgaggg

SEQ ID NO: 56 - DNA, 23 bp, Artificial Sequence
  Variable sequence of PPO9 sgRNA sg851 including PAM site
ggtttggtcggcgtctttccagg

SEQ ID NO: 57 - DNA, 20 bp, Artificial Sequence
  Variable sequence of PPO8 sgRNA1 sg852
atcagggtaagaaaaatgga

SEQ ID NO: 58 - DNA, 20 bp, Artificial Sequence
  Variable sequence of PPO8 sgRNA2 sg853
cacatcgcggcggtcgagct

SEQ ID NO: 59 - DNA, 20 bp, Artificial Sequence
  Variable sequence of an alternative PPO1 sgRNA
caagctacgctacgagtacc

SEQ ID NO: 60 - DNA, 20 bp, Artificial Sequence
  Variable sequence of an alternative PPO1 sgRNA
cgcgaactcggatcttgacg

SEQ ID NO: 61 - DNA, 20 bp, Artificial Sequence
  Variable sequence of an alternative PPO1 sgRNA
gccaaaggcggcgaggacca

SEQ ID NO: 62 - DNA, 20 bp, Artificial Sequence
  Variable sequence of an alternative PPO1 sgRNA
gaggggttggttgtggtctc

SEQ ID NO: 63 - DNA, 19 bp, Artificial Sequence
  Variable sequence of an alternative PPO1 sgRNA
cggagggaaggggcatgaa

SEQ ID NO: 64 - DNA, 20 bp, Artificial Sequence
  Variable sequence of an alternative PPO2 sgRNA
aggactgcttggagaccgat

SEQ ID NO: 65 - DNA, 20 bp, Artificial Sequence
  Variable sequence of an alternative PPO2 sgRNA
gtacgtgtagcgcagccaat

SEQ ID NO: 66 - DNA, 20 bp, Artificial Sequence
  Variable sequence of an alternative PPO2 sgRNA
ggtcacggtcaaggactgct

SEQ ID NO: 67 - DNA, 21 bp, Artificial Sequence
  Variable sequence of an alternative PPO2 sgRNA
agagccagctgttgtggactg

SEQ ID NO: 68 - DNA, 20 bp, Artificial Sequence
  Variable sequence of an alternative PPO2 sgRNA
ttccagaaagggagcgagag

SEQ ID NO: 69 - DNA, 21 bp, Artificial Sequence
  Variable sequence of an alternative PPO2 sgRNA
tgaacagcctaaccctggcgg

SEQ ID NO: 70 - DNA, 20 bp, Artificial Sequence
  Variable sequence of an alternative PPO2 sgRNA
aacagcctaaccctggcgcg

SEQ ID NO: 71 - DNA, 20 bp, Artificial Sequence
  Variable sequence of an alternative PPO3 sgRNA
ggtacttgtagcgcagccac

SEQ ID NO: 72 - DNA, 20 bp, Artificial Sequence
  Variable sequence of an alternative PPO3 sgRNA
gtacttgtagcgcagccacc

SEQ ID NO: 73 - DNA, 19 bp, Artificial Sequence
  Variable sequence of an alternative PPO3 sgRNA
gttaggcttgtcgcccccg

SEQ ID NO: 74 - DNA, 20 bp, Artificial Sequence
  Variable sequence of an alternative PPO3 sgRNA
aggactgcttcaagacccgg

SEQ ID NO: 75 - DNA, 20 bp, Artificial Sequence
  Variable sequence of an alternative PPO3 sgRNA
acggctgagcttcttccaca

SEQ ID NO: 76 - DNA, 19 bp, Artificial Sequence
  Variable sequence of an alternative PPO4 sgRNA
atggaagtcatccacaatg

SEQ ID NO: 77 - DNA, 19 bp, Artificial Sequence
  Variable sequence of an alternative PPO4 sgRNA
cgtttcgggtcgtacagcg

SEQ ID NO: 78 - DNA, 20 bp, Artificial Sequence
  Variable sequence of an alternative PPO5 sgRNA
ttgcaatgtcgaccgcatgt

SEQ ID NO: 79 - DNA, 19 bp, Artificial Sequence
  Variable sequence of an alternative PPO5 sgRNA
cgtttcgggtcgtacagcg

SEQ ID NO: 80 - DNA, 19 bp, Artificial Sequence
  Variable sequence of an alternative PPO6 sgRNA
ggccggtcgccgcacccgg

SEQ ID NO: 81 - DNA, 20 bp, Artificial Sequence
  Variable sequence of an alternative PPO6 sgRNA
acggcgtgacatgccaccag

SEQ ID NO: 82 - DNA, 20 bp, Artificial Sequence
  Variable sequence of an alternative PPO7 sgRNA
aatccccgtcactactggat

SEQ ID NO: 83 - DNA, 20 bp, Artificial Sequence
  Variable sequence of an alternative PPO7 sgRNA
gtccaagtgccacgatgcca

SEQ ID NO: 84 - DNA, 20 bp, Artificial Sequence
  Variable sequence of an alternative PPO7 sgRNA
cgttgtcttcgaactcaatg

SEQ ID NO: 85 - DNA, 20 bp, Artificial Sequence
  Variable sequence of an alternative PPO7 sgRNA
tcgagcaagttcctctccca

SEQ ID NO: 86 - DNA, 20 bp, Artificial Sequence
  Variable sequence of an alternative PPO7 sgRNA
cgttgtcttcgaactcaatg

SEQ ID NO: 87 - DNA, 20 bp, Artificial Sequence
  Variable sequence of an alternative PPO7 sgRNA
tgcggttaccgcggaggttc

SEQ ID NO: 88 - DNA, 20 bp, Artificial Sequence
  Variable sequence of an alternative PPO7 sgRNA
actacctccacttctacgag

SEQ ID NO: 89 - DNA, 19 bp, Artificial Sequence
  Variable sequence of an alternative PPO7 sgRNA
ctggaagtcatccacaatg

SEQ ID NO: 90 - DNA, 20 bp, Artificial Sequence
  Variable sequence of an alternative PPO8 sgRNA
gtcacccctccgtgtccgcc

SEQ ID NO: 91 - DNA, 20 bp, Artificial Sequence
  Variable sequence of an alternative PPO8 sgRNA
cgcctggatgggatttccga

SEQ ID NO: 92 - DNA, 20 bp, Artificial Sequence
  Variable sequence of an alternative PPO8 sgRNA
taatcgtaacgaagccactc

SEQ ID NO: 93 - DNA, 20 bp, Artificial Sequence
  Variable sequence of an alternative PPO8 sgRNA
ccggctgcaagcagtccttg

SEQ ID NO: 94 - DNA, 20 bp, Artificial Sequence
  Variable sequence of an alternative PPO9 sgRNA
acgagaatgtgcgcctgcgc

SEQ ID NO: 95 - DNA, 20 bp, Artificial Sequence
  Variable sequence of an alternative PPO9 sgRNA
acatagacaaactcaggtac

SEQ ID NO: 96 - DNA, 20 bp, Artificial Sequence
  Variable sequence of an alternative PPO9 sgRNA
tgcccacgagaacaagcacg

SEQ ID NO: 97 - DNA, 20 bp, Artificial Sequence
  Variable sequence of an alternative PPO9 sgRNA
ggatctcccgagagaagctc

SEQ ID NO: 98 - DNA, 137 bp, Artificial Sequence
  Scaffold used with the variable sequences of the sgRNA
  misc_feature (1)..(10): Can be C, G, T or A
  misc_feature (1)..(10): Can be C, G, T or A according to variable sgRNA
  misc_feature (11)..(20): n is a, c, g, or t
nnnnnnnnnnnnnnnnnnnngttttagagctagaaatagcaagttaaaataaggctagtccgttatcaacttgaaaaagtggcaccgagtcggtgctttttttctagacccagctttcttgtacaaagttggcatta

SEQ ID NO: 99 - DNA, 23 bp, Artificial Sequence
  Variable sequence of PPO8 sgRNA sg852 including PAM site
atcagggtaagaaaaatggaagg

SEQ ID NO: 100 - DNA, 23 bp, Artificial Sequence
  Variable sequence of PPO8 sgRNA sg853 including PAM site
cacatcgcggcggtcgagcttgg

SEQ ID NO: 101 - DNA, 23 bp, Artificial Sequence
  Variable sequence of an alternative PPO1 sgRNA including PAM site
caagctacgctacgagtaccagg

SEQ ID NO: 102 - DNA, 23 bp, Artificial Sequence
  Variable sequence of an alternative PPO1 sgRNA including PAM site
cgcgaactcggatcttgacgagg

SEQ ID NO: 103 - DNA, 23 bp, Artificial Sequence
  Variable sequence of an alternative PPO1 sgRNA including PAM site
gccaaaggcggcgaggaccaagg

SEQ ID NO: 104 - DNA, 23 bp, Artificial Sequence
  Variable sequence of an alternative PPO2 sgRNA including PAM site
aggactgcttggagaccgattgg

SEQ ID NO: 105 - DNA, 23 bp, Artificial Sequence
  Variable sequence of an alternative PPO2 sgRNA including PAM site
gtacgtgtagcgcagccaatcgg

SEQ ID NO: 106 - DNA, 23 bp, Artificial Sequence
  Variable sequence of an alternative PPO2 sgRNA including PAM site
ggtcacggtcaaggactgcttgg

SEQ ID NO: 107 - DNA, 23 bp, Artificial Sequence
  Variable sequence of an alternative PPO3 sgRNA including PAM site
ggtacttgtagcgcagccaccgg

SEQ ID NO: 108 - DNA, 23 bp, Artificial Sequence
  Variable sequence of an alternative PPO3 sgRNA including PAM site
gtacttgtagcgcagccaccggg

SEQ ID NO: 109 - DNA, 23 bp, Artificial Sequence
  Variable sequence of an an alternative PPO7 sgRNA including PAM site
aatccccgtcactactggatcgg

SEQ ID NO: 110 - DNA, 23 bp, Artificial Sequence
  Variable sequence of an alternative PPO7 sgRNA including PAM site
gtccaagtgccacgatgccaagg

SEQ ID NO: 111 - DNA, 23 bp, Artificial Sequence
  Variable sequence of an alternative PPO7 sgRNA including PAM site
cgttgtcttcgaactcaatgcgg

SEQ ID NO: 112 - DNA, 23 bp, Artificial Sequence
  Variable sequence of an alternative PPO7 sgRNA including PAM site
tcgagcaagttcctctcccatgg

SEQ ID NO: 113 - DNA, 23 bp, Artificial Sequence
  Variable sequence of an alternative PPO7 sgRNA including PAM site
cgttgtcttcgaactcaatgcgg

SEQ ID NO: 114 - DNA, 23 bp, Artificial Sequence
  Variable sequence of an alternative PPO7 sgRNA including PAM site
tgcggttaccgcggaggttccgg

SEQ ID NO: 115 - DNA, 23 bp, Artificial Sequence
  Variable sequence of an alternative PPO8 sgRNA including PAM site
gtcacccctccgtgtccgcccgg

SEQ ID NO: 116 - DNA, 23 bp, Artificial Sequence
  Variable sequence of an alternative PPO8 sgRNA including PAM site
cgcctggatgggatttccgaggg

SEQ ID NO: 117 - DNA, 23 bp, Artificial Sequence
  Variable sequence of an alternative PPO8 sgRNA including PAM site
taatcgtaacgaagccactccgg

SEQ ID NO: 118 - DNA, 23 bp, Artificial Sequence
  Variable sequence of an alternative PPO8 sgRNA including PAM site
ccggctgcaagcagtccttgagg

SEQ ID NO: 119 - DNA, 23 bp, Artificial Sequence
  Variable sequence of an alternative PPO9 sgRNA including PAM site
acgagaatgtgcgcctgcgcagg

SEQ ID NO: 120 - DNA, 23 bp, Artificial Sequence
  Variable sequence of an alternative PPO9 sgRNA including PAM site
acatagacaaactcaggtaccgg

SEQ ID NO: 121 - DNA, 23 bp, Artificial Sequence
  Variable sequence of an alternative PPO9 sgRNA including PAM site
tgcccacgagaacaagcacgagg

SEQ ID NO: 122 - DNA, 23 bp, Artificial Sequence
  Variable sequence of an alternative PPO9 sgRNA including PAM site
ggatctcccgagagaagctccgg

SEQ ID NO: 123 - DNA, 21 bp, Artificial Sequence
  Forward primer to detect a gene editing event in PPO1
cgagaaccacggtatcgatct

SEQ ID NO: 124 - DNA, 20 bp, Artificial Sequence
  Reverse primer to detect a gene editing event in PPO1
atgctgagtgaagttccggg

SEQ ID NO: 125 - DNA, 21 bp, Artificial Sequence
  Forward primer to detect a gene editing event in PPO2
acaagagaactccagcacgta

SEQ ID NO: 126 - DNA, 20 bp, Artificial Sequence
  Reverse primer to detect a gene editing event in PPO2
aggggcctgaatggggttag

SEQ ID NO: 127 - DNA, 20 bp, Artificial Sequence
  Forward primer to detect a gene editing event in PPO3
gccaattcttctccaacggc

SEQ ID NO: 128 - DNA, 20 bp, Artificial Sequence
  Reverse primer to detect a gene editing event in PPO3
ccttggtgagcttgggagtc

SEQ ID NO: 129 - DNA, 20 bp, Artificial Sequence
  Forward primer to detect a gene editing event in PPO8
gatagaagacgccatgccca

SEQ ID NO: 130 - DNA, 20 bp, Artificial Sequence
  Reverse primer to detect a gene editing event in PPO8
gaagccgatctggtcgtagg

SEQ ID NO: 131 - DNA, 22 bp, Artificial Sequence
  Forward primer to detect a gene editing event in PPO9
ctttgatttgcaacgatctcgg

SEQ ID NO: 132 - DNA, 21 bp, Artificial Sequence
  Reverse primer to detect a gene editing event in PPO9
tcatacttggccacgaactcc

SEQ ID NO: 133 - DNA, 20 bp, Artificial Sequence
  Primer 1684 used to confirm absence of Cas9
agtacaaggtgccgagcaaa

SEQ ID NO: 134 - DNA, 20 bp, Artificial Sequence
  Primer 1685 used to confirm absence of Cas9
acctgaatgcagtggtaggc

SEQ ID NO: 135 - DNA, 20 bp, Artificial Sequence
  Primer 1686 used to confirm absence of Cas9
ccgtgctgttcttttgagcc

SEQ ID NO: 136 - DNA, 20 bp, Artificial Sequence
  Primer 1687 used to confirm absence of Cas9
ggtggccttgcctatttcct

SEQ ID NO: 137 - DNA, 20 bp, Artificial Sequence
  Primer 1563 used to confirm absence of backbone
acacacgaagcagcagatca

SEQ ID NO: 138 - DNA, 20 bp, Artificial Sequence
  Primer 1564 used to confirm absence of backbone
acagcttgcggtacttctcc

SEQ ID NO: 139 - DNA, 20 bp, Artificial Sequence
  Primer 1565 used to confirm absence of backbone
aacccagacaacagcgatgt

SEQ ID NO: 140 - DNA, 20 bp, Artificial Sequence
  Primer 1566 used to confirm absence of backbone
ccgtcaatgtatccggcgta

SEQ ID NO: 141 - DNA, 20 bp, Artificial Sequence
  Primer 1567 used to confirm absence of backbone
agacacgccagatcaccaag

SEQ ID NO: 142 - DNA, 20 bp, Artificial Sequence
  Primer 1568 used to confirm absence of backbone
agaagcctccggtctgtact

SEQ ID NO: 143 - DNA, 20 bp, Artificial Sequence
  Primer 1569 used to confirm absence of backbone
acaagcctggggataagtgc

SEQ ID NO: 144 - DNA, 20 bp, Artificial Sequence
  Primer 1570 used to confirm absence of backbone
cgttcggtcaaggttctgga

SEQ ID NO: 145 - DNA, 20 bp, Artificial Sequence
  Primer 1571 used to confirm absence of backbone
catccagaaattgcgtggcg

SEQ ID NO: 146 - DNA, 20 bp, Artificial Sequence
  Primer 1572 used to confirm absence of backbone
atgacccgacaaacaagtgc

SEQ ID NO: 147 - DNA, 20 bp, Artificial Sequence
  Primer 1573 used to confirm absence of backbone
ctcgtgaccaccctgaccta

SEQ ID NO: 148 - DNA, 20 bp, Artificial Sequence
  Primer 1574 used to confirm absence of backbone
gtccatgccgagagtgatcc

SEQ ID NO: 149 - DNA, 20 bp, Artificial Sequence
  Primer 1575 used to confirm absence of backbone
ggcggacaagtggtatgaca

SEQ ID NO: 150 - DNA, 20 bp, Artificial Sequence
  Primer 1576 used to confirm absence of backbone
ggcggtgctacagagttctt

SEQ ID NO: 151 - DNA, 2839 bp, Musa acuminata
atggcttctatctcgcagctaatcactacaagcatccccaccaccttttctctctcctattcatgccccttccctccgagaaccacggtatcgatctccggcttcaaacacctccaccacgtttcccccatctcatgctccaccagagaccacaaccaacccctcgtcgatcgccgccacgtcctcgtcggaataggcagcctctacggcgcctccgctgcactaacgtccctccgcgaggccagtgcggcaccgatcgcggcgcccgacctgtccgcatgcgggctggctgacctccctccggatgccactccgacgaactgctgcccgccgtccgcgggagacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttcttccagtgataagcagcagattgatcacaacctcaaggttatgtacaggcaggtaattaatggactccaataccactgaatcagtcatgcattatgttattatctgtacgagcatcataaaataatgattgataggcgacatcagccataattatcctgcattaattgacgaatcttgtaaacaatgtcatcttgttgggctttccatggctcatggagttgatcgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcagtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagtgatatgacaaaaaatggcagacccagctcctggcgacgtcacccgcacgtggacaggcgcggcgcccctggagggcaataaacgtgacggtctgtgcccggacgtcagcctcactgagcccacagccccctcgggtgacccagcacaaccggacgagacagaagcaggtaacggttgaagctcgcccataccctgcgatccctcggtctcccacgcagccccagtggcaatgtcagacgccaccaaaggtacagtcctgccctacagacagctacgtcgggtaacatcaaacctcctctataaataccccgaagccctgaacaaaaaggggatcacacactgaacacacggaggtttctcctcctcctaaaccccctccacgttgctaacttgatcgtcggaggggtcgggccgagctcccggtccgacctgtgtgcaggtgagagacggtgtcgcctcttcccggtgctgcggcggagctgcctcccgacccgagctaccgacccgacccgccgacccgagttgccgacctgaaccaccgtgctcggccgccaggagaccccgaggagcgcccccacagagatcaccgccattcggaccctgaaccaagccgcgacggccccgacgccatggctaaacagttacttaccgtaacaacaagcatgacaagatgagcaagcagctgaagaccaggctgcagctgggcttgactgagctgttggaggatctcaaggctgatgggagcatcatggtgactttggtgccgaggcaggggaaggggaaggtgaaggttggcagtctcaagatcgagttagttgattgagcatgggagacttccattcccgcgcgtcgttagggtttggaataagggcaacatgtttactgggacacctcccagaataagagagctttcacttgtgtgacttcaaaatcctggccatcttctatggatt

SEQ ID NO: 152 - DNA, 1935 bp, Musa acuminata
aaagcgacgaccatggccggccttccttattcagctcctcaccctgccaccatctccgcttcctccaactcctttgcatgccccttccgcagcaaggggcttgtcttcccctaccctaccagaagagcactccatgttcgtcccaacatcgcatgcaaggcaggcgaggagcacgagatcgctgctaaggtcgaccgacgcgacgtactcgtgggcctcggtgggctctgcggagccgccgctggccttggcgggttcgataaagccgccctcgctaaccccattcaggcccctgatctctccaagtgcggccctgccgacctccccaccggcgtgccagtcgtcaactgctgcccgccctaccgtcccggtgcgaagattgtggatttcaagcggccgtcgccgtcctccccactccgcgtccgccccgccgcccacttggttgaccccgagtacctggccaagtacaagaaggccatcgagctcatgaaggcgctcccggccgatgaccctcgcaacttcatgcagcaggccgacgtccactgcgcctactgcgacggcgcttacgaccagatcggcttccccaaccttgagatccaagtccacaacagctggctcttcttcccctggcaccgcttgtacctctacttcaacgagaggatcctcggcaagctcatcggcgacgacaccttctcgctccctttctggaactgggacgcacccggcggaatgatgctgccttcgatctacgccgatccttcgtcacccctctacgacaaacttcgcgacgccaagcaccaacctcctgtccttgtcgacctcgactacaatggaaccgacccaaccttccccgacgcccagcaaatcgatcacaacctcaagatcatgtaccgccaagtcttctccaacggcaagacgccgttgctgttcttaggctcagcttaccgtgccggtgaacagcctaaccctggcgcgggctccgtcgagaacatgccgcacaacaacgtgcacttgtggaccggcgaccgcacccagcccaacttcgagaacatgggcaccttctacgccgcggcgcgcgaccccatcttcttcgcccaccacgccaacatcgaccgcatgtggtacctgtggaagaagctcagcaggaagcaccaggacttcaatgactcggactggctcaaagcttccttccttttctacgacgagaacgccgacttagttcgggtcacggtcaaggactgcttggagaccgattggctgcgctacacgtaccaagacgtgaagatcccatgggtgaacgcccgaccgactcccaagctcgccaaggcgaggaaagccgccagcagttcgctgaaagccaccgcggaggtgcagttccctgtgacgctggaatccccggtcaaagcgacggtgaagaggcccaaggtggggaggagcggcaaggagaaggaagatgaggaggagatactcatagtggaggggatcgagttcgaccgcgactacttcatcaagttcgacgtcttcgtgaacgcgacggagggcgacggcatcacggccggggccagcgagttcgccggcagcttcgtgaacgtcccgcacaagcacaagcaccgcaaggatgagaataagctgaagacgaggctgtgtcttggaatcaccgacctgctcgaggacatcggcgcggaggacgacgacagcgtgctcgtcaccatcgtgccgaaggcgggcaaaggaaaggtgtccgtcggcggtcttcggattgacttttccaagtgaggaaataaaagaattcacgtgccgtgcttgctttctatgtacgaataaaataagagtgcatcatcaccgaccatggttctactttaaacttcgtgttgtacgtttgatgtcagtagtctctcattgttgttgtttacagtttctatggacatgcatttg

SEQ ID NO: 153 - DNA, 10192 bp, Musa acuminata
atggcagagatcggcaatccaaatgaaaacaacacctcaatatttgccgaccgcaagccgccgaaacgcctcgtgttgccattcggcgtccgaccaccagtggcaggtcatgtgaagcaaatatattctgtgtggtcatatacaagtcattcttcacctctctgtctttatataatctaattttctagtttatactattctgttaagacaaattaagattattatttcagctgacagagaattgcagactgtgtgttttctttgacatggagtttggaacatattaggctaccaaattcttctaaccaaatccaccaagcatggaatctaccaaccaacatccaataaattaaagaactacttcatcacctaatacttatggctcatcttggagatgtggatctgcaaacaagcatattgaaacagtgccatggagatcaagaagtttacagtaatcttggttgcaccaatgacgatgcttcttttcttgaggtaaatttatctcaaagcagagtaatggtgacttgtttctcattccctttgttcttccaggatcttcacttgcaagggaaatcttggagcagctttgttaggtagcataagagacaacaggagatctttacgtcagtgagctgctatcttagattctgtcgcaagattctgtcctgctagtagagcagcatttggcaccaaatcaagtctttggtgtttgtcctcaggctggaagctgctgtaagttccaaagctaacaccaagtcacataactttggtagatatctaagaaaagggcataaataacagagaacttaacatgaactctatgaacaaaatttgaactttaacgttaacgaggaggaagaagaagagatctctggtagtaaacatatctatgtccctgccaaagattttgccaatgaagctcaagcagcattgtcggcctcgagcatatgttattgtagaagtggagttcgaattctttcatgagttaagtgaaggaagagaccaagagtggcttaagataattgtaccacttttatgccgagaccctcagcgttgttgggaccgctcaacacattagagcgtctgatatattgtataaagacatctgagctcgaaaagcgattgtatgctccgctgggtcaaagttgtcgttgaaggtttccaatgatgggagacaaaagtttacagacatcagctcctcctagaggctttggacgaaaagggagcggcctgaggtggggtcatctgcatcttcccttctggattattagaactctcgatgcatcttgtccatgtgttggtccatctatcatagttaggctctcaaggagtcgttcattgaatctatcaattaggtcttcgattcaagtaatgttgggccctagtgcaatggcacactgaactccactattgggaaggaggatgctcttgtcagtagttcagttggtcgagtgcagtcctgggttgccagtactagatcaatcggggaaaccctggtgggcgctagtgctaatagtggttgagacaccgatcgtggttgtaattgcaccatagtctattgagctagctgaggcagaagttgggcgatagcctacatcatccccatgagcatttgcatttggtgagtgaggttcaataatatatcgatgaggacaactaggtaccttgcattcatccctagaggtgagagtttggggttgttgaataggtgccagtaatgccccagtgtttgagtggaggtggatgcattcaccagaggaacggtgggcaattgctcgccttgtgtgaaagtaatatggattgggataaggcctcatcgctggagtactcgttaagagagttcatgggtagatatttttgcaacattcagggccctccttctaacgccaaaatgttgtagggagagtaccgtcgattgtgagtcaacacgacttggttttgactcaaaggtgtagggtcatccgattaatcctcgtactcatatgatgaagcggttgatggggcaccaaggcatcgaggtggtcgtcgggtagaatatgaatgtccaaggtggtcttgagggaaggttgaggtggagttcgactcctcgatgggtttctgaataaaggttagtgttaggaagagtttcctgaattgaccctccaatgctcaagttagctttagagtggtatatagtgtatgagactatttgcagagatattcgactcttggatcagcttttatatctgactggtcgagagatttatttgtaacaactataatgccctctcatttgtccatggaggccaaccaaggtggggtcggggcaggcccgaacaatctctcgtggacagttgtccacggaggccaactgggctagtccgaatggtctcttgttgactattatccatgggggctaataagccataactatctccatgtcaccgcctcaggtgactcaaccttcaacttccttgtcagcatatctatcattataaaaagatatatatttgtgtcccagtcgactttgtcaaaactctcatagcttagcttgctggtgttgtctacctaaacaaaacatatgtatttgtttaggtagacaacaccatttctgagcaagtttagacaggatgaggaacccaaatttctgagcaagtgcaagaattctcaatattgaaaagaagaggaggaggattactacctgaagcaaataggaagaagaacggcaaagtgatggccacaccccttgtgtccattcttgaagaccaccttctacctgcaagaaattcttagtgtgctcactagatttctggtattgtcaccaggagttagggtgttgttttagttgttttcttgtcatgggaaaacgcattgcttacatagcaacgttttggacttgcttgattgtgtcggcgcactagatgttttgcagcttgagcagatacatcatatgcaaaacaagggtcattttggacacattatctacaaataaaatactttaggattgatataatagtggaggaggcatggctgacaccttaacatcatatgtgatcgatactttagcattatgtggattaacaccttaacatcgcatcatcgataccttagcaccacgtatctcaacatgagacacctcatcaccgtatgaccaacacatagtcagcatgagattgacaattggatgataatgaaaagattgggttttagatgaacttgcaatcccttgtgatcaacattgatcacaagaataccagtccatagagcccactataaaagagctctccaagtgtgttccctatatacatttaaacatcgcttttaagcttggttcgaatccttccaaacccttgaaatgagacaaatgaggaggaaggtctcggttagctccgaatcgacatccctcgatgatgatgcaacctcggctcaaaagcatctcttgatagttgtgcaagttgaattgacctagaagcgtctcttagtaacaacgtaggctaaattggcttagaagcgtttctcgacaatgatgtggtcgaatcggtccaaagcgctcaattcgatgatagcataatcaaaatgacccaaagcattttgtccaacgatagtgcaatcaaaacaacataaaacattctcatcaatgataacatagttgaaatgacttgtagtgctccatttgaggatgctgaacaaaatggtctaaagtgcttaattcgatgacaatgcagccaaaagaacccaaaagtacttcctttaacgatagcataactaaaacaacccaaagtgctcacttcgataacaatgtaatgaaaatattccaaagtgctatatttaacgatagcacaatcgaaatggctcaaaatgttccctatgacgatagtgcaatatatacaacctaaagcgctccttttggtgataagatcataaacccttttttttagtttggtagttaagtaggagttatatccttgactccactgcacttatcgaggcatgctatgggaataatgatataaggacgatcgatacctcaatgtcatcttagcaccatatggctaacacttaacaccacatgatgggcacttcacaaccacgtggttgaaacctcatcaccacatggccaacatatcacaactacatggcatatatctcaacattgcatgactaacacattataaccacgtattcaacacctcacagctatgtggtcgacatcttaccgctacatgaccgacaacttgacattagatgggtgatgaggtgattgactcgaccttgcgaaaattgatacacataatttgatacaaccatgttcgctggtttcagcgcttcatgatcgcatgtggttggagaaaatgcataatcattccaattattaattatgacctcaagctaatggtcaaataccatattataaaatgctcccaagtatgttctaagggggctctcccctctatgttgaattacacttttcttatttgacatttgactaaattctttgaacctcttactgatttaagtatcataagagctttgttgggtatacctttaacgtagttttttttttttttaggatcccttatcgaacccactcattgcccgatatcaaatttttgatagacttggacatcaacaaaggttgcctccaaaattctccccatgtattggacaaccaaattacattttcataccttccaagttgtgattttggagaataataatttaatatcttctcacatatccaattttgtatgaaaaataaacttaatttagtaacttttcttttatttgaaaacacgaattagtaaaaacaaatctgcactgaaaatttctcaatagtcaaacctcttaatttgcttcaaaacatagaaattatcaattattttgtattggatacgaagaataatattcttagtaataagattctttaatcttaaacttgatcaattattgaatatatatatatatatatatatatatatatatatatgtatatatatgtttgaatgttcatggttttgatagatctgttgttgtttacttgaacaaaacataccctaagtcctcgtttcaataccaagaagatagtgagcacactaaagattgcttgtaggttgaatgtgatggtggttttcaagattggacagaagggatgcagccatcactttgatcttcctattttgtttactgtataaaaattattattattttcctatacatgagtttcataatgaattggtatgctttcatttcatgttaaattattgatatttgtatatattttatgaatattagatattatttttatattttttaaaaattactatcaacatgtgtggttgttgatgcttctttataatatatttatttttagagtagtttactactttctaatatgtctaaggtttggatagaggagacttttcagaaacactataaaaaaggcatgatttttttagttaaaaaaaagttactattataaagatagagatttgaattcaaagataaaaaagagagtggcttctataaattatgaataatgaaataatagtttatttttttataaatctaggatgtgataagatcgatttggtctataccactattgggagatgcttttgaatcgagaaggtcgcatcgtggttgagggacgctttatggctaaatttagttgcaatgtgcttcttgatcaagtttagttactttgtcatcaaggaatgatttatgaccaagttcaatcacgttattatataaggatgcttctttattgattcgatcatgttattgtagagggttgattttggctgggtttggtcacctagttattaagaattatcttttcaagtagttcaatcaggttaatgttaggagtcacctcttcaaaaagtttagtcatgttggtgttgggagctacttcttttggtcatgttggtgttataagcgatctctttaagcagtttaactatgttgggataaggaaccacctctttcgaggctcgaatagtacggtgtggagagtcacttttttaggtggttcgaccatattaatataaggaaccacttctttggggaatctatcattatagtgaagggagctacctcatttatgttggtgttgctcaattatgtctaactaccattttatcctattagagggtggtataatgcaactacagtcctcaattatgttacttgttatagaatttgatcattcgataaatgaagaaatttcggagccacaaatctgaatttgtcacaaactagacatagtgaatttggaaagagatcctataaaaaagaattatgttggaggcatactcgataaggcccctccgtgcttaacacaataaagagttctaaattcactacaagagatccccctaaagaacctcctccgggcctttttgtagtgtggtatttggctactgggttgatgtcatggttagtaatcaaaatggttttacgcttcttccagccacatatgtgattgtatagtgccgaatccgagtaggaaacaaagagtcgctgctggtgtgtgataatgtcgcagctgtgtaaagtagagaacaacaagcagtggctctcacccatggcgtccatgctgcatgcagggcattgtggtattcgtcgatattgagacaggttgacttcgatgcggaagttgatgttgaactgtgttcgggtgtcattatggagtgccatgtgttaatctgtgcccgtgcgaacagtgacttgcagggttgagcctaaaggcttaaatgggatcaatgcatctcttttgcacacaccgagaggcttaagagcaatgttcaaatatgtttataggaaacacacctagcgtgccttttatagtgggctctatgaatcatttttcttatgattgatgttgatcatgaggggattgtgcatttatcttggacctaatcattctcatcccgtcgctggatgtaatgaaaccttgaagattgacttgatcttatcattgtcatctaattgttggtcttgtgtcgatcgcgtgtcagtcatgtggtgctgaggtgtttctccacatcatgaatggtcgttgcgcacccgtaacatctttgttaatgaaccgttcgtcacttttgtgcacttgtatagttgtttgacagtatgttttgtattagttttgtatatttttacttgaaaattatgagcaagaatagattgaaatattacaaagcgattgctcaccatgatgagtaaaacttccacaaaatagattcgtttttgtgtgtcacaacttgtttttttcaagctatggtgcgatgcaaaatatcaactatattagtatcctggaaccccttaggtggcacctggttagataaagatttgttgttgtgttgggcgtcaaatagatttcacaagttcgcatgttaagtctatgagaacttgccatcgtgctcgacatcctatggaaaatcatttaaggacttgtaattgctcggttgccttctaaagtccttggtttcaccattctctcttgcatatcgagtgtttgatgaattacttgtaaagttgccgtcttcgtaagatgttaggacttgttcgtcgttctatttcgaactaatcaatttgctcttttataggttctatgagacctgagcaaggttgtagctaggcttatcatttgcagatagataatgcaataaagcttcataacttaaataatcctagttaagtctatgacattgacgtaagggtgcttcatggcttagacaacttcagttaagttcataatattagcgtaattatgcctcatgacttaaatagttctaattaagtcggtgatattgtcgtttcaaaaaagttgatgataacgtggtgttgagatatcaatcacatgatattgaggtatcagccatgcgatgctaaagtatcaatcctcatagcattaatgtgtcgactccacaatgttgaagtgtcaaccatgcctccactcgtatatcaattcttaagtatctctttgcgtctaaaatcgaaaaacattggaagtgtctgatgcgaccaagcaaatccaaaatgtccccttttacatatgaatgatgtgcccgcagaagatgcaaaacataaagcatcgacatgtggaatccttggtatatgttacgtgagctggttataatagcattaatccttgccagatacccttaatcttccatttatgctcctacgtgcatgaacactgtaactccatggtggcatcagatatatctttcacttggataccttgtatcttatctactactaaccatctttgcattcatgagattttctactgctacatatggtcgatcgatgcaaattataaataacaacataccctaactcctggtgacagtaccagaaatctagtgagcacactaaggatttcttgcaggtagaaggtggtcttcaagtatggacacaaggggtgtggccatcactttggtgttgctgttcttcttcctatttgcttcaggcagtaatcctcctcctcctcttcttttcaatatcgagagttcttgcacttgctcagaaattagggttcctcatcctgtctaaactttagtatcagaaatggagaaaaggaataaatttgttttcattattactttatataaatctataacaacagatctatatgaacattcaaaacaaacaaacacacacacacacagacacacaggagcactgatggtcttcttcaacagaggcactgacaaaggttgatgcaagtttctgcgaccccaagaacaacaatgagtgtataaacttaggatcccaaggaggcgagtgcgccgccaactgctgccttcccatccctcccggtgcgaagattgtggatttcaagcggccatcgcggtccacccccctccgcgtccgccccgccgcccacttggtcgaccccgagtacctggccaagtacaagaaggccatcgagctcatgaaggcgctcccggccgacgaccctcgcaacttcatgcagcagtccaacgtccactgcgttcactgcgacagcatccccgacaatgacatccaagtccaccagaactggttcttctacccctggcaccgctggtacctctacttcaacgagaggatcctcggcaagctcatcggcgacgacaacttcacgctccctttctggaactgggactcgctcggcggaatgatgctgccgtcgatctacgccgacccttcgtcgcccctctacgacaaccttcgcgacgccaagcaccaacctcctttccttgtcgacttcgacttcaatggaaccgacccaggcttcaccgacgcccagcaaatcgatcacaacctcaagatcatgtaccgccaattcttctccaacggcaagaagccgatgctgttcttaggctcaccttaccgcgggggcgacaagcctaaccccggcgggggctccgtcgagaacacgccgcacaacaacgtgcacacgtggaccggcgaccgtactcgtcccaacttcgaggacatgggcaccttctactcggcggggcgcgaccccatcttcttcgcccaccacgccaacatcgatcgcatgtggtccctgtggaagaagctcagccgtaagcaccgggacttcaatgactcggactggctgaaaacttccttcctcttctacgacgagaacgccgacttagttcgggtcacggtcaaggactgcttcaagacccggtggctgcgctacaagtaccaagacgtggagatcccatgggtgaaagcccgaccgactcccaagctcaccaaggcgaggaaagccgccagcggatcgctgaaacccaccgcggaggcgcagttccctgtgacgctggaatccccggtcagcgcgacgctgaagaggcccaaggtggggaggagccgcaaggagaaggaagaggaggaggaagtgctcatagtggaggggatcgagttcgaccgcgaccagttcatcaagttcgacgtcatcgtgaacgcgacggagggcgatggcatcacgcccgcggacagcgagttcgccggcagcttcgtgaacaccccgcacaggcacaggcacctcaaggaggagaacaaggggacgacgaggctgtgtctggggatcaccgacctgctcgaggacatcggcggggaggccgacgacggcgtgctcgtcaccatcgtacccaaggcaggcaagggcaaggtttccgtcggcggtcttcggattgacttcaccaagtga

SEQ ID NO: 154 - DNA, 2229 bp, Musa acuminata
ccctgccctctgtatgagctcagtgttctttttaaggagtaccagtgaagagctgctcatcaccggccagtggttttccctgtagactcccacgccacccctctctctctctggtccactgaacagtagtagacatgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgccgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgaccccggcgagacgatctcggagtacagcttccccgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaataccgacgacgtgcccatccaggtccacttcagctggatcttcctcccatggcaccgctactacctccacttctacgaaaggatcctcggcaagctcatcgacgacgacaccttcaccatcccattctggaactgggacaccaaggacggcatgacgttccccgccatcttccaggatgcggcatccccgctgtacgacccgaaacgcgaccaacgccacgtcaaggacggcaagatcctcgacctcaagtacgcctacaccgaaaacactgcatccgacagcgagatcatacgggagaacctctgcttcatacagaagacgttcaagcacagcctgtcgctggcggaactgttcatgggggatcccgtgcgcgcgggggagaaggagatccaggaggctaatgggcagatggaagtcatccacaatgcggcgcacatgtgggtcggagagccggacggatacaaggaaaacatgggggacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgaacagctcgtcaaagtcaaggcaagtcagatgctgtctgcaatttatacccatccctgcaaatgcaatagaaacgttgtatacataactgtcgtataccgtgcagatgagcgactgcctcaacccgaccaagcttcggtacacgttcgaacaagttcccctcccatggctgggcaaaatcaattgccagaagacggcagagacgaagtccaaggccacgacggagctgtcgctgacgcgcgtgaacgaattcgggacgacggcccaggcactcgacgcgagcaacccgctgcgggtgatcgtggcaaggccgaagaagaaccgcaagaagaaggagaagcaagagaaggtggaggtgattcagatcaaggatattcaggtgaccaccaacgagacagctcgcttcgacgtctacgtcgcggttccttacggtgacctcgccggacccgactacggcgagttcgcgggcagctacgtgaggctggcgcataggatgaagggaagcgacgggaccgcagagcaggtccccaagaagaagggaaagctcaagctgggtattacgccgctgctcgaggacatcgatgctgaggacgccgacaagttggtggtcaccctggttctccgcaccgggagcgtcaccgtgggcggagtttccatcaatctcctgcagacagattctaccgccgccatctaaatgatggcctcggatcacagcttctccccgcttaagttggagtgatcgattactggtgctgcttccttcctccctgtcgttgttgctatcttcttgatctggaacgatccttcaataattagggcatgacagtagtcgtcgcccgatcccatatgtacgtgttgctgtcaacagctgtacatgtgacgttgtggtgtgactatatattttattgcggtcatcgttgtttctttctta

SEQ ID NO: 155 - DNA, 1858 bp, Musa acuminata
catggtggtcctttcgttgtaaaggctactcaacaacgggggttaaacgaggaagtgcgctctgcatgatgcttatccagacgccctctgtctgagctcagtgttctatttaaggagtatcagtgaagagttgctcaacaccggccagtggtcagtgctcgctccatttgctggcccctcctttccctgtagactcccacgccacccctctctctctctctctctctggtctactgaacagtagtagacatgtccctgctgttgaactctagcttcaccggtgcttcctctgcatgcctcctccaacgggaaaggtcccgccgccgccgcctccacgtccctggcgtgacatgccgccagggcagtaatggtgaccgcagcgatgccgcccgccagcagcagtcgccgctgctgctggatcggcgcgacatgctgttgggtttaggagggctttacggcgtgaccgcaggacccaaggttctggcggcgccgataatgccgccggatctgtccaagtgctaccctgccaccgcacctgccctcgacaacaaatgctgcccgccttacgtccccggcgagacgatctcggagtacagcttccctgctacgcccctccgggtgcggcggccggcccatatcgtgaaggacgatcaggagtatatggacaagtacaaggaggcagtgaggaggatgaagaatctgccggcagaccacccttggaactactaccagcaggcgaacatccactgccagtattgcaactacgcctaccaccagcaaaatgccgacgacgtgcccatccaggtccacttcagctggatcttcctcccatggcaccgctactacctccacttctacgaaaggatcctcggcaagctcatcgacgacgacaccttcaccatccccttctggaactgggacaccaaggacggcatgacgttccccgccatcttccaggatgcggcatccccgctgtacgacccgaaacgcgaccaacgccacgtcaaggacggcaagatcctcgacctcaagtacgccatcaccgaaaatgaaagcactgcatccgacagcgagatcatacgggagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacagttgggtcggagagccgagcggaaactatgaagacatgggcgacttctccaccgccgcccgcgattctgttttcttctgccaccattgcaatgtcgaccgcatgtgggacatctaccgcaacctccgcggcaaccgcgtcgagttcgaagacaacgactggttggacagcaccttcctcttccacgacgagaacgagcagctcgtcaaagtcaaggcaagtcagatgctgtctccaatttatacccatccctgcaaatgcaatagaaacgttgtatacataactgtcgtataccgtgcagatgagggactgcctcaacccgaccaagcttcggtacacgttcgagcaagttcccctcccatggctgggcaaaatcaattgccagaagacggcagagacgaagtccaaggccacgacggagctgtcgctgaatcgcgtgaacgaattcggaacgacggcccaggcactcgacgcgagcaacccgctgcgggtgatcgtggcaaggccgaagaagaaccgcaagaagaaggagaagcaagagaaggtggaggtgattcagatcaaggatattaaggtgaccaccaacgagacagctcgcttcgacgtctacgtcgcggtcccttacggtgacctc

SEQ ID NO: 156 - DNA, 2004 bp, Musa acuminata
aacactagtagatatgtctctcctgttgaactctagcctcaccggagcttcctctgcatgcctcctccgtcgagaaaagtgccgccgccgcggccgcggtcacgtccacggcgtgacatgccaccaggggggtaatgatgaccgcagagaggccgcccggcagcagcggtcccggttgctgctggatcggcgcgacatgctgttgggggggttaggagggctttacggcgtgaccgcagggcccaaagttctggcggagccgataatgccgcctgatctgtccaagtgccacgatgccaacgcacctgccctcgacaaccactgctgcccgccttacagtggcagcgagacgatcttggagtacgacttccccgctacgcccctccgggtgcggcgaccggcccacctcgtgaaggatgatcaggagtatatggacaagtacaaggaggccgtgaggaggatgaagaacctgccggcagaacacccttggaactactaccagcaggcgaacatccactgccagtattgcaacgacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccaggatgcggcatccccgctgtacgacccgaaacgcgaccaacgtcacgtcaaggacggcgcgatcctcgacctcaagtacgcctacaccgaaaacactgcatccgacagcgagatcatacgggagaacctctgcttcatacaaaagacgttcaagcacagcctgtcgctggcggagctgttcatgggggatcccgtgcgcgcgggggagaaggagatccaggaggcaaacgggcagctggaagtcatccacaatgcggcgcacatgtgggtcggagagccggacggatacaaggaaaacatgggcgacttctccaccgcggcccgcgattctgttttcttctgccaccattccaatgtcgaccgcatgtgggacatctaccgcaacctccgcggcaacagcgtcgagttcaacgacaaagactggttgcacagcaccttcctcttccatgacgagaacgagcagctcgtcaaagtcaaggcaagtcagatgctgtctcgaatatatacccatccctgcaaatgcaatagaaacgttgtacacataactgttgtatactgtgcagatccaggactgccttaacccgaccaagcttcggtacacgttcgagcaagttcccctcccatggctgggcaatataaattgccagaagacggcagagacgaagtccaagtccacggcagagctgtcgctgaagcgggtgggcgaattcgggacgacacccaaggcgctcgacgcgagcaacccgctgcgggtgatcgtggcaaggccgaagaagaaccgcaagaagaaggagaagcaagagaaggtggaggtgctccagatcaaggatattaaggtgaccaccaacgagacagctcgcttcgacgtctacgtcgccgttccttacggtgacctcgccggacccgactacggcgagttcgtgggcagcttcgttaggctggcgcataggaagaagggaagcgacgggaccgaagagcagggccccaagaagaagggaaagctcaagctgggtattacggcgctgctcgaggacatcgatgctgaggacgccgacaagttggtggtcaccctggttctccgcaccgggagcgtcactgtgggtggagtttccatcaaactcctgcagacagatactcccgccgtcatctaaatgatgccctcagatcacagcttctcccctcttaagttggagtgatcgtgtactggttcttcttccttcgtccatgtcgttgttgcgttccttcaacaagttgggtatgacagtagcagtcgcccgt

SEQ ID NO: 157 - DNA, 1628 bp, Musa acuminata
gctagtagacatggctctccagttgaactctagcttcaccggagcttcctctgcatgcctcctccatcgggaaaggtcccgccgcctcaacgtccctgtcgtgacatgccgccaggggaataatgatgatcgcagcgatgccgctcgccagcagaaatccccgtcactactggatcggcgcgacatgctgctggggttaggagggctttatggcttgaccgcaggacccaaagttctggcgaagccgataatgccgcctgatctgtccaagtgccacgatgccaaggcacctgccctcgacaaccactgctgcccgccttacaaccccagcgagacgatctcggagtacggcttccccgctacgcccctccgggtgcggcggccggcccacctcgtgaaggacgatcaggagtatttggacaagtacaaggaggccgtgaggaggatgaagaatctgccggcagaccacccttggaactactaccagcaggcgaacgtccactgccagtactgcaactacgcctactaccagcaaaataccgacgacgtgcccgtccaggtccacttcagctggatcttcctcccctggcaccgctactacctccacttctacgagcggatcctcggcaagctcatcgacgacgacaccttcaccatccccttctggaactgggacaccaaggacggcatgacgttccccgccatcttccacgaagagtcatccccgctgtctgacacgaaacgcgaccaacgccacgtcaaggatggcaagatcgtcgacctcaagtacgcctacaccgaaaaccctgcctccaacagcgagatcattcgagagaacctctgcttcatacagaagacgttcaagcacagcctgtcgctggcggagctgttcatgggggatcccgtgcgcgcgggggagaaggagatccaggaggctaacgggcagctggaagtcatccacaatgcggtgcacatgtgggtcggagagccgtgcggatacaaggaaaacatgggcgatttctctaccgccgcccgcgattctgttttcttcagccaccactccaatgtcgaccgcttgtgggaaatctaccggaacctccgcggtaaccgcattgagttcgaagacaacgactggttggacagtaccttcctcttctacgacgagaacgagaagctcgtcaaagtcaaggcaagtcatatgctgccccgacaatatacccatccctgcaaatgtaacagaaccgtacatatatgtatatatatatatatatacgtaactgttgtatactgtgcagatgggggactgcctcaacccgaccaagcttcggtatacgttcgagcaagttcctctcccatggctgggcaaaattaattgccagaagacgacagagacgaagtccaagtccacgacagagatgtcgctgacgcgcgtgggagaattcgggacgacgcccaaggcgctcgacgcgagcaacccgctgcgggtgatcgtggcaaggccgaaaaagaaccgcaagaagaaggagaagcaagagaaggtggaggtgcttcagatcaatgatattaaggtgaccaccaacgagacagctcgcttcgacgtctacgtcacggttccttacggtgacctc

SEQ ID NO: 158 - DNA, 1952 bp, Musa acuminata
ctcccacatcaaccacagcaaaactatagcaataccaagttatcgatcccaatggtcagccttcctaaagctactcttcctctctcctccctctcccctccctccaactccaactccaactccaactccaactcctttgcatgcgccttccatttttcttaccctgatagaagacgccatgcccactccaagatctcatgcaaagctagcgatgagcatgagatgactgcaaatgccaagctcgaccgccgcgatgtgctcgttggcctcggcgggctttgtggagccgctgccggcctcgggatcgacggtaaggccctcggaaatcccatccaggcgcctgatcttaccaagtgcggccccgccgatctacccaaaggtgcaacgcccaccaactgctgcccgccttattttcccgacaagaagattatcgatttcaagcgtccgccgaattcgtcacccctccgtgtccgcccggccgcccacttggtcgactccgactacctggacaagtataagaaggcggtggagctcatgagagcactgccggccgacgatccgcgcaacttcatgcagcaggccaatgtccactgcgcttactgtgatggcgcctacgaccagatcggcttccccaacctcgagctccaagtccacaactcctggctcttcttcccttggcaccgcttctacctctacttccacgagaggatcctcggcaagctcataggcgacgacactttcgcccttcctttctggaactgggacgcgcccggcggcatgaagctgccgtcgatctacgccgatccttcgtcctcgctctatgacaagtttcgcgacgccaagcaccagccgccggtcctcgtcgacctcgactacaacggaaccgaccctagtttcaccgacgcagagcagatcgatcagaacctcaagatcatgtaccggcaggtgatctccaacggcaagacgccgttgctgttcttaggctcggcttaccgtgccggcgacaacccaaaccccggcgcgggctcgctcgagaacataccacacggccccgtccacgggtggactggcgacagaaaccaacccaatctcgaggacatgggcaacttctactccgcggggcgcgaccctatcttcttcgcccaccattcaaacgtcgaccgcatgtggtacttgtggaagaagctcggcgggaagcatcaggactttaacgataaggactggctcaacaccaccttcctcttctacgacgagaatgctgacttagttcgagtcaccctcaaggactgcttgcagccggagtggcttcgttacgattaccaagacgtcgagatcccgtggctgaagacccggccgactcccaaagccttgaaggcgcagaaaaccgcagcgaaaacactgaaagctacagcagagacgccgttcccggtgacgctgcaatccgcggtgagcacgacggtgaggaggcccaaggtatcgaggagcggcaaggagaaggaagaggaagaggaggtcctcatcgtggaggggatcgagttcgaccgcgactacttcataaagttcgacgtcttcgtgaacgccaccgagggtgagggcatcacgccgggcgccagcgagttcgcgggcagcttcgtcaacgtcccgcacaagcacaagcacagcaagaaggagaagaagctgaagacgaggctctgcctggggatcactgacctgctcgaggacatcggggcggaggacgacgacagcgtgctcgtcaccatcgtcccgaaagccggaaagggcaaggtgtcggtcgccggcctccgcatcgatttcccaaattgaagtaatactatatatttctactacctatcaaggaaaataaaagccgcaccatcgaaacagtgtaaagctcaactttaatcgtcgagttacatctgcggacgtcgtaatgttgcgttatggaccggaaa

SEQ ID NO: 159 - DNA, 3788 bp, Musa acuminata
gagctaatagaggttacaatagcatttcggcagctcaacaaagccgggccagcgtgtgtggtgaagtgtgagcacgtggtttgaattgtatgaacagcataccaaccgtgtttctctcctcctcttcgtcttcgtcttccttcttttccttgtggtacacgatgcatcggcctttaaaacagactgaccatcctggtttcctcctcttcatgtaggcaagcagctagcagcactagaatacatctctctagcccagctttctcttcatcatcggaagctttggcaagacaccatccaacatctgcttgctctcgaggatcgccttcattgatctctgggaaaaggggaggaatccagtaagtaatcatcagcaacgccaagaaccgaagctgcttgtggttgattcgttctgctttgggtttccttctcccgacagtgacggcgtctttcttccctcttctgtgggtagcaggagtgtaaacagatggagggcaaacgatggttgtctcttctcctcctcgtgcttgttctcgtgggcatctccatggatctcccgagagaagctccggcggcttcttctaatatcttgaagagttcatctgccaggtatgctaatcactttgtgtttcttttccgagacacatgtttgaatcttgcaagtttcttgttcgaagcatatcttgttgttgttgatgttacatgtttcatggaccgccactcgaattaagcctccgattccagtcaagaaatatgcagatgtacaataagatcatagcatcatctgaggcattgtccgtgacagaaaggttatcactggattttagagactttgatatcgcacacacgtgaaacacgaggctctgatttgatggatcgacacgtcctataccaggggactggccaatcaatcaaagccaatggggcccgcccgtcggggaaaccaacatcgaaatcacacgtcagtgagccccggggttggtttatggtgggtggatgtgccacgtattatgagtggaaacggtgcgagtcttagccatgcgttccgtcacattatagccggctcacgtatgccgcagtgaacgggcaaatgcgccgatgcgatggtatagaaaagggtatagagtcggtgttgagatttttgtgcacaaagaatgattcaaactgagggttctgttacttggaaattttcttcatgagtagattgctgcattttagtgtcttttactttgagctaaaatgacttgtcaacttggtatagaaatcaacaacaaaaaaacaaaatattttagagcaggtatatttttatctatcttgatttgttggtatagaaatcaaaaaaaaaaaaaaaaagacttgccacaggacatgacactttcgcttacttgtttaagccgaatcaaagactcgcctagtcgccttggtggccatctgagtcatcatagtgagccggcttggtggctactagttagtcgccttgacggctttctgagtcatcgcttggatagcgaaccggcttggtggctaccagtttgtcgccatctcggttgatcaaacggcatcggacactcacatcaaaagggtacatgactgacgtctgtgttcgagtcatcttaaatcattcatccgattcggatcaccacatggtaagggtcagcgattggtatgtcaatcattgatgggctcgctgtcctgcaatgatcgacgatggtgggccacgtgagaggaattgaatattgggtgtcgtgggccacgttccaaagtcggtgaacgtgcccggagcagtaacggatgtgggatccatggcgtgacaagtatggctttcttaagggaaaaataaagaaaaagaatttccccatcatacgttggtggttcagttccttgtgttcttagttcgtatttagctgaagaaacaaggtataatgtaatcgaataatgtgaatcctatatatgtggggtgaaatgctatatattctctttgatttgcaacgatctcggatccaaaatgctgaaatggagtttgaacttgcagaataccagtgaatcctcaaggcggagaacagagagatggaagcaagagcaagggcattcccctcaaagcaaacctatcggtttgccatgcttcattctcggacgccgatcgtccggtctactgttgcccggcctggaaagacgccgaccaaaccttgctcgacttcgagttcccggatccgtcgtcgccggtgcgtatccgacggcctgcccatctcgtcgacgaggagttcgtggccaagtatgagagggcggtggccatcatgaagcagatcccgcctgaccatccccacaacttttggcgccaggccaacatgcactgcctctactgcaccggcgcctacgaccagatgaactcctctgccctcttcaagatccacaggtcatggctcttcttcccctggcaccgagccttcatctatttccacgagcgcatcctcgggaagttcatgggagacgacaccttcgcgctcccctactggagctgggacacccccgagggcatgtggttccccgacatctaccggaagggagctctgaatgagacggagcgcgacgccattcacctacgggaggccgccgtcgatgacttcgactacgtggatcatgacctcgccagcgacgtgcagatcgccgacaacctcgcgttcatgtaccaccagatgatctcgggagcgaagaagaccgagctgttcatgggttgcaagctgcggtccggcgtcgaggggtggtgtgatgggcccgggacgatcgaagcggcacctcacaacacgttgcacagctgggtagggaacaggtacaaccccgaaagagagaacatgggggcgttctactccgccgcgcgagacgaagtgttcttcgcgcaccactccaacatcgaccgcatgtggaccgtgtggaagaagctgcacggcgacaagccggagttcgtcgaccaggagtggctcgagtcggagttcaccttctacgacgagaatgtgcgcctgcgcaggatcaaggtgcgcgacgtgttgaacatagacaaactcaggtaccggtacgaagacatcgacatgccatggctcgctgcacgtcccaagccttccgttcaccctaagatcgcgcgcgacatattgaagaagcgcaatggcgaaggcgtactgagaatgcccggcgaaacggatcgttcacaactctccgaatatggtagctggacactggacaagaccatcaccgtgagggttgacaggccaaggatcaacaggacagggcaagaaaaagaggaagaagaggagatcttattggtctacggaatcgatactaagagaagcagattcgtcaaattcgatgtgttcatcaacgtcgtcgacgaaaccgtgctgagcccaaagtcgagggagttcgcagggaccttcgtcaatctccaccacgtctcgaggacgaaaagccatgacgatggcggcatggattcgaagatgaaaagccaccttaagctcggtatatcggaacttttggaagacctcgaggcagacgaagatgacagcatctgggtgacactggtgccaagaggcggcacgggggtcaacaccaccgtggacggcgtccggatcgactacatgaagtagtgaaccagcacgccgctcctcccctccccatcagaagtggtataatatttatattggattgaggctcgtggtatcttttgataagagtaagttccataaatttagaagaagaatcatgttctttatttatattaaattaatgtgatttggccatca

SEQ ID NO: 160 - DNA, 359 bp, Artificial Sequence
  TaU6 promoter
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcaga

SEQ ID NO: 161 - protein, 60 aa, Artificial Sequence
  Ma10_p20510-PPO9 from Figure 3
GDKPEFVDQEWLESEFTFYDENVRLRRIKVRDVLNIDKLRYRYEDIDMPWLAARPKPSVH

SEQ ID NO: 162 - protein, 60 aa, Artificial Sequence
  Ma08_p09180-PPO7 from Figure 3
GNRIEFEDNDWLDSTFLFYDENEKLVKVKMGDCLNPTKLRYTFEQVPLPWLGKINCQKTT

SEQ ID NO: 163 - protein, 60 aa, Artificial Sequence
  Ma08_p09170-PPO6 from Figure 3
GNSVEFNDKDWLHSTFLFHDENEQLVKVKIQDCLNPTKLRYTFEQVPLPWLGNINCQKTA

SEQ ID NO: 164 - protein, 59 aa, Artificial Sequence
  Ma08_p09150-PPO4 from Figure 3
GNRVEFEDNDWLDSTFLFHDENEQLVKVKMSDCLNPTKLRYTFEQVPLPWLGKINCQKT

SEQ ID NO: 165 - protein, 60 aa, Artificial Sequence
  Ma08_p09160-PPO5 from Figure 3
GNRVEFEDNDWLDSTFLFHDENEQLVKVKMRDCLNPTKLRYTFEQVPLPWLGKINCQKTA

SEQ ID NO: 166 - protein, 60 aa, Artificial Sequence
  GPO3_21_US9580723B2_21 from Figure 3
TKNKDINDKDWLDTGFLFYDENAELVRVTVRDTLDNKKLGYTYEDVEIPWLKSRPTPRRT

SEQ ID NO: 167 - protein, 60 aa, Artificial Sequence
  PPO3_BAA21676 from Figure 3
GKRADLTDSDWLDSGFLFYNENAELVRVKVRDCLETKHLGYVYQDVDIPWLSSKPTPRRA

SEQ ID NO: 168 - protein, 60 aa, Artificial Sequence
  APO5_AAA69902 from Figure 3
GKRTDLTDSDWLDSGFLFYNENAELVRVKVRDCLETKNLGYVYQDVDIPWLSSKPTPRRA

SEQ ID NO: 169 - protein, 60 aa, Artificial Sequence
  PPO7_BAA21677 from Figure 3
GKRADLTDSDWLDSGFLFYNENAELVRVKVRDCLETKNLGYVYQDVDIPWLSSKPTPRRA

SEQ ID NO: 170 - protein, 60 aa, Artificial Sequence
  Ma06_p31080-PPO1 from Figure 3
SRRKDLADPDWLDASFVFYDENANLVKIRVRDCIDSDKLRYEYQDVGNPWLNTRPTVTSG

SEQ ID NO: 171 - protein, 60 aa, Artificial Sequence
  Ma07_p03650-PPO3 from Figure 3
RKHRDFNDSDWLKTSFLFYDENADLVRVTVKDCFKTRWLRYKYQDVEIPWVKARPTPKLT

SEQ ID NO: 172 - protein, 60 aa, Artificial Sequence
  Ma07_p03540-PPO2 from Figure 3
RKHQDFNDSDWLKASFLFYDENADLVRVTVKDCLETDWLRYTYQDVKIPWVNARPTPKLA

SEQ ID NO: 173 - protein, 60 aa, Artificial Sequence
  Ma08_p34740-PPO8 from Figure 3
GKHQDFNDKDWLNTTFLFYDENADLVRVTLKDCLQPEWLRYDYQDVEIPWLKTRPTPKAL

SEQ ID NO: 174 - protein, 240 aa, Artificial Sequence
  Ma07_g03540-PPO2-WT from Figure 6
ATGGCCGGCCTTCCTTATTCAGCTCCTCACCCTGCCACCATCTCCGCTTCCTCCAACTCCTTTGCATGCCCCTTCCGCAGCAAGGGGCTTGTCTTCCCCTACCCTACCAGAAGAGCACTCCATGTTCGTCCCAACATCGCATGCAAGGCAGGCGAGGAGCACGAGATCGCTGCTAAGGTCGACCGACGCGACGTACTCGTGGGCCTCGGTGGGCTCTGCGGAGCCGCCGCTGGCCTTGGC

SEQ ID NO: 175 - protein, 150 aa, Artificial Sequence
  Ma07_g03450-PPO2-GE-pool-embryos from Figure 6
ATGGCCGGCCTTCCTTATTCAGCTCCTCACCCTGCTCGTCCCAACATCGCATGCAAGGCAGGCGAGGAGCACGAGATCGCTGCTAAGGTCGACCGACGCGACGTACTCGTGGGCCTCGGTGGGCTCTGCGGAGCCGCCGCTGGCCTTGGC

SEQ ID NO: 176 - DNA, 23 bp, Artificial Sequence
  Variable sequence of PPO1 sgRNA sg857 including PAM site
gaggggttggttgtggtctctgg

SEQ ID NO: 177 - protein, 127 aa, Artificial Sequence
  PPO1 mutant protein sequence
MASISQLITTSIPTTFSLSYSCPFPPRTTVSISGFKHLHHVSPISCSTRDHNQPLVDRRHVLVGIGSLYGASAALTSLREASAAPIAAPDLSACGLADLPPDATPTNCCPPSAGDATEFVIPDRPRP

SEQ ID NO: 178 - DNA, 1736 bp, Artificial Sequence
  PPO1 mutant coding sequence
atggcttctatctcgcagctaatcactacaagcatccccaccaccttttctctctcctattcatgccccttccctccgagaaccacggtatcgatctccggcttcaaacacctccaccacgtttcccccatctcatgctccaccagagaccacaaccaacccctcgtcgatcgccgccacgtcctcgtcggaataggcagcctctacggcgcctccgctgcactaacgtccctccgcgaggccagtgcggcaccgatcgcggcgcccgacctgtccgcatgcgggctggctgacctccctccggatgccactccgacgaactgctgcccgccgtccgcgggagacgcgaccgagttcgtcattcccgaccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttcttccagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcagtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggctgatgggagcatcatggtgactttggtgccgaggcaggggaaggggaaggtgaaggttggcagtctcaagatcgagttagttgattga

SEQ ID NO: 179 - DNA, 2838 bp, Artificial Sequence
  PPO1 mutant gene sequence
atggcttctatctcgcagctaatcactacaagcatccccaccaccttttctctctcctattcatgccccttccctccgagaaccacggtatcgatctccggcttcaaacacctccaccacgtttcccccatctcatgctccaccagagaccacaaccaacccctcgtcgatcgccgccacgtcctcgtcggaataggcagcctctacggcgcctccgctgcactaacgtccctccgcgaggccagtgcggcaccgatcgcggcgcccgacctgtccgcatgcgggctggctgacctccctccggatgccactccgacgaactgctgcccgccgtccgcgggagacgcgaccgagttcgtcattcccgaccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttcttccagtgataagcagcagattgatcacaacctcaaggttatgtacaggcaggtaattaatggactccaataccactgaatcagtcatgcattatgttattatctgtacgagcatcataaaataatgattgataggcgacatcagccataattatcctgcattaattgacgaatcttgtaaacaatgtcatcttgttgggctttccatggctcatggagttgatcgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcagtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagtgatatgacaaaaaatggcagacccagctcctggcgacgtcacccgcacgtggacaggcgcggcgcccctggagggcaataaacgtgacggtctgtgcccggacgtcagcctcactgagcccacagccccctcgggtgacccagcacaaccggacgagacagaagcaggtaacggttgaagctcgcccataccctgcgatccctcggtctcccacgcagccccagtggcaatgtcagacgccaccaaaggtacagtcctgccctacagacagctacgtcgggtaacatcaaacctcctctataaataccccgaagccctgaacaaaaaggggatcacacactgaacacacggaggtttctcctcctcctaaaccccctccacgttgctaacttgatcgtcggaggggtcgggccgagctcccggtccgacctgtgtgcaggtgagagacggtgtcgcctcttcccggtgctgcggcggagctgcctcccgacccgagctaccgacccgacccgccgacccgagttgccgacctgaaccaccgtgctcggccgccaggagaccccgaggagcgcccccacagagatcaccgccattcggaccctgaaccaagccgcgacggccccgacgccatggctaaacagttacttaccgtaacaacaagcatgacaagatgagcaagcagctgaagaccaggctgcagctgggcttgactgagctgttggaggatctcaaggctgatgggagcatcatggtgactttggtgccgaggcaggggaaggggaaggtgaaggttggcagtctcaagatcgagttagttgattgagcatgggagacttccattcccgcgcgtcgttagggtttggaataagggcaacatgtttactgggacacctcccagaataagagagctttcacttgtgtgacttcaaaatcctggccatcttctatggatt

SEQ ID NO: 180 - DNA, 20 bp, Artificial sequence
  PPO1 sgRNA ID 857 from Table 8, without PAM
gaggggttggttgtggtctc

SEQ ID NO: 181 - DNA, 20 bp, Artificial sequence
  PPO1 sgRNA ID 858 from Table 8, without PAM
cctcaagggcgaggacgggt

SEQ ID NO: 182 - DNA, 19 bp, Artificial sequence
  PPO1 sgRNA ID 2019 from Table 8, without PAM
agggacgttagtgcagcgg

SEQ ID NO: 183 - DNA, 20 bp, Artificial sequence
  PPO1 sgRNA ID 3518 from Table 8, without PAM
caagctacgctacgagtacc

SEQ ID NO: 184 - DNA, 20 bp, Artificial sequence
  PPO1 sgRNA ID 3519 from Table 8, without PAM
gccaaaggcggcgaggacca

SEQ ID NO: 185 - DNA, 20 bp, Artificial sequence
  PPO1 sgRNA ID 4117 from Table 8, without PAM
gcgattgccagcgatgtacg

SEQ ID NO: 186 - DNA, 20 bp, Artificial sequence
  PPO1 sgRNA ID MolMoClo0157 from Table 8, without PAM
gtacagccaagtcggcttcc

SEQ ID NO: 187 - DNA, 20 bp, Artificial sequence
  PPO1 sgRNA ID MolMoClo0158 from Table 8, without PAM
agagccatgagttgtgcacc

SEQ ID NO: 188 - DNA, 20 bp, Artificial sequence
  PPO1 sgRNA ID MolMoClo0159 from Table 8, without PAM
tcgccggtccagacgtggac

SEQ ID NO: 189 - DNA, 20 bp, Artificial sequence
  PPO1 sgRNA ID MolMoClo0160 from Table 8, without PAM
cgagccagtcggggtcggcc

SEQ ID NO: 190 - DNA, 20 bp, Artificial sequence
  PPO2 sgRNA ID 854 from Table 8, without PAM
aggaagcggagatggtggca

SEQ ID NO: 191 - DNA, 20 bp, Artificial sequence
  PPO2 sgRNA ID 855 from Table 8, without PAM
atgcgatgttgggacgaaca

SEQ ID NO: 192 - DNA, 20 bp, Artificial sequence
  PPO2 sgRNA ID 3520 from Table 8, without PAM
ggtcacggtcaaggactgct

SEQ ID NO: 193 - DNA, 20 bp, Artificial sequence
  PPO2 sgRNA ID 2961 from Table 8, without PAM
agagccagctgttgtggact

SEQ ID NO: 194 - DNA, 20 bp, Artificial sequence
  PPO2 sgRNA ID 2962 from Table 8, without PAM
gttccagaaagggagcgaga

SEQ ID NO: 195 - DNA, 20 bp, Artificial sequence
  PPO2 sgRNA ID 2963 from Table 8, without PAM
tgaacagcctaaccctggcg

SEQ ID NO: 196 - DNA, 20 bp, Artificial sequence
  PPO2 sgRNA ID 2964 from Table 8, without PAM
gaacagcctaaccctggcgc

SEQ ID NO: 197 - DNA, 20 bp, Artificial sequence
  PPO3 sgRNA ID 1435 from Table 8, without PAM
ggttaggcttgtcgcccccg

SEQ ID NO: 198 - DNA, 20 bp, Artificial sequence
  PPO3 sgRNA ID 1436 from Table 8, without PAM
aggactgcttcaagacccgg

SEQ ID NO: 199 - DNA, 20 bp, Artificial sequence
  PPO4 sgRNA ID 4118 from Table 8, without PAM
atgtcgcgccgatccagcag

SEQ ID NO: 200 - DNA, 20 bp, Artificial sequence
  PPO4/PPO5 sgRNA ID 4119 from Table 8, without PAM
tgcggtcaccattactgccc

SEQ ID NO: 201 - DNA, 20 bp, Artificial sequence
  PPO6 sgRNA ID 4120 from Table 8, without PAM
tcggcgcgacatgctgttgg

SEQ ID NO: 202 - DNA, 20 bp, Artificial sequence
  PPO6 sgRNA ID 4121 from Table 8, without PAM
ggctttacggcgtgaccgca

SEQ ID NO: 203 - DNA, 20 bp, Artificial sequence
  PPO7 sgRNA ID 3521 from Table 8, without PAM
aatccccgtcactactggat

SEQ ID NO: 204 - DNA, 20 bp, Artificial sequence
  PPO7 sgRNA ID 3522 from Table 8, without PAM
gtccaagtgccacgatgcca

SEQ ID NO: 205 - DNA, 20 bp, Artificial sequence
  PPO7 sgRNA ID 3523 from Table 8, without PAM
tcgagcaagttcctctccca

SEQ ID NO: 206 - DNA, 20 bp, Artificial sequence
  PPO7 sgRNA ID 3524 from Table 8, without PAM
tgcggttaccgcggaggttc

SEQ ID NO: 207 - DNA, 20 bp, Artificial sequence
  PPO8 sgRNA ID 3525 from Table 8, without PAM
gtcacccctccgtgtccgcc

SEQ ID NO: 208 - DNA, 20 bp, Artificial sequence
  PPO8 sgRNA ID 3526 from Table 8, without PAM
cgcctggatgggatttccga

SEQ ID NO: 209 - DNA, 20 bp, Artificial sequence
  PPO8 sgRNA ID 3527 from Table 8, without PAM
taatcgtaacgaagccactc

SEQ ID NO: 210 - DNA, 20 bp, Artificial sequence
  PPO8 sgRNA ID 4158 from Table 8, without PAM
ggtaagatcaggcgcctgga

SEQ ID NO: 211 - DNA, 20 bp, Artificial sequence
  PPO8 sgRNA ID 4159 from Table 8, without PAM
catgaagttgcgcggatcgt

SEQ ID NO: 212 - DNA, 20 bp, Artificial sequence
  PPO8 sgRNA ID MolMoClo0161 from Table 8, without PAM
agctccaagtccacaactcc

SEQ ID NO: 213 - DNA, 20 bp, Artificial sequence
  PPO8 sgRNA ID MolMoClo0162 from Table 8, without PAM
cgtcccagttccagaaagga

SEQ ID NO: 214 - DNA, 20 bp, Artificial sequence
  PPO8 sgRNA ID MolMoClo0163 from Table 8, without PAM
taccggcaggtgatctccaa

SEQ ID NO: 215 - DNA, 20 bp, Artificial sequence
  PPO8 sgRNA ID MolMoClo0164 from Table 8, without PAM
gtcgccagtccacccgtgga

SEQ ID NO: 216 - DNA, 20 bp, Artificial sequence
  PPO9 sgRNA ID 850 from Table 8, without PAM
aaccgataggtttgctttga

SEQ ID NO: 217 - DNA, 20 bp, Artificial sequence
  PPO9 sgRNA ID 851 from Table 8, without PAM
ggtttggtcggcgtctttcc

SEQ ID NO: 218 - DNA, 20 bp, Artificial sequence
  PPO9 sgRNA ID 3528 from Table 8, without PAM
acgagaatgtgcgcctgcgc

SEQ ID NO: 219 - DNA, 20 bp, Artificial sequence
  PPO9 sgRNA ID 3529 from Table 8, without PAM
tgcccacgagaacaagcacg

SEQ ID NO: 220 - DNA, 20 bp, Artificial sequence
  PPO9 sgRNA ID 4160 from Table 8, without PAM
caggccgggcaacagtagac

SEQ ID NO: 221 - DNA, 20 bp, Artificial sequence
  PPO9 sgRNA ID MolMoClo0165 from Table 8, without PAM
ggcctggcgccaaaagttgt

SEQ ID NO: 222 - DNA, 20 bp, Artificial sequence
  PPO9 sgRNA ID MolMoClo0166 from Table 8, without PAM
gggggtgtcccagctccagt

SEQ ID NO: 223 - DNA, 20 bp, Artificial sequence
  PPO9 sgRNA ID MolMoClo0167 from Table 8, without PAM
tcggggaaccacatgccctc

SEQ ID NO: 224 - DNA, 20 bp, Artificial sequence
  PPO9 sgRNA ID MolMoClo0168 from Table 8, without PAM
atcacaccacccctcgacgc

SEQ ID NO: 225 - DNA, 20 bp, Artificial sequence
  PPO9 sgRNA ID MolMoClo0169 from Table 8, without PAM
cgaccaggagtggctcgagt